(19) United States
(12) Patent Application Publication
(10) Pub. No.: US 2025/0032632 A1
(43) Pub. Date: Jan. 30, 2025

(54) DRUG CONJUGATES OF cMET MONOCLONAL BINDING AGENTS, AND USES THEREOF

(71) Applicant: MITSUBISHI TANABE PHARMA CORPORATION, OSAKA (JP)

(72) Inventors: Julia CORONELLA, Carlsbad, CA (US); Marco GYMNOPOULOS, San Diego, CA (US); Vincent BLOT, Seattle, WA (US); Ryo FUJITA, Osaka (JP); Roland NEWMAN, San Diego, CA (US)

(21) Appl. No.: 18/800,527

(22) Filed: Aug. 12, 2024

Related U.S. Application Data
(63) Continuation of application No. 17/042,013, filed on Sep. 25, 2020, now Pat. No. 12,060,427.
(60) Provisional application No. 62/649,078, filed on Mar. 28, 2018.

(51) Int. Cl. A61K 47/68; A61K 31/5517; A61K 39/00; A61K 47/64; A61P 35/00; C07K 16/28

(52) U.S. Cl. CPC A61K 47/6849; A61K 31/5517; A61K 47/6415; A61K 47/68035; A61P 35/00; C07K 16/2863; A61K 2039/505

(57) ABSTRACT

Presented herein are novel monoclonal cMET binding agents that are conjugated to pyrrolobenzodiazepine toxins, composition thereof and uses thereof for the treatment of cancer.

### RELATED APPLICATIONS

This patent application is a continuation of U.S. patent application Ser. No. 17/042,013 filed Sep. 25, 2020, entitled DRUG CONJUGATES OF cMET MONOCLONAL BINDING AGENTS, AND USES THEREOF, and naming Julia Coronella, Marco Gymnopoulos, Vincent Blot, Ryo Fujita and Roland Newman as an inventors, and designated by attorney docket no. 057774-0514861, which is a national phase filing of, and claims the benefit of, International Patent Application No. based on PCT/JP2019/013345 filed on Mar. 27, 2019, entitled DRUG CONJUGATES OF cMET MONOCLONAL BINDING AGENTS, AND USES THEREOF, and naming Julia Coronella, Marco Gymnopoulos, Vincent Blot, Ryo Fujita and Roland Newman as an inventors, which claims the benefit of U.S. Provisional Patent Application No. 62/649,078 filed on Mar. 28, 2018, entitled DRUG CONJUGATES OF cMET MONOCLONAL BINDING AGENTS, AND USES THEREOF, naming Julia Coronella, Marco Gymnopoulos, Vincent Blot, Ryo Fujita and Roland Newman as inventors, and designated by attorney docket no. 057774-0453744. The entire content of the foregoing patent application is incorporated herein by reference, including all text, tables and drawings.

### SEQUENCE LISTING

This application contains a Sequence Listing which has been submitted electronically in WIPO Standard ST.26 (XML format) and is hereby incorporated by reference in its entirety. Said Sequence Listing copy, created on Oct. 21, 2024, is named “057774-0580729_CORRECTED_Sequence_Listing_ST26.xml” and is 126,976 bytes in size.

### FIELD OF THE INVENTION

Embodiments of the invention relate to cMET binding agents that are conjugated to pyrrolobenzodiazepine toxins, composition thereof and uses thereof.

### BACKGROUND

The protein cMET, sometimes called MET or hepatocyte growth factor receptor (HGFR), is a protein that in humans is encoded by the MET gene (MET proto-oncogene, receptor tyrosine kinase). cMET is a single-pass cell surface receptor that possesses tyrosine kinase activity. The primary single chain precursor protein of the MET translation product is post-translationally cleaved to produce an alpha and a beta subunit, which are disulfide linked to form a mature cell surface cMET receptor. cMET is expressed on cells of epithelial origin, as well as stem cells, progenitor cells and other cell types (e.g., various cancer cell types). Hepatocyte growth factor/Scatter Factor (HGF/SF) and its splicing isoforms (NK1, NK2) have been identified as ligands of cMET.

cMET is thought to be essential for normal embryonic development, organogenesis and wound healing. Abnormal cMET expression and/or activity is associated with certain neoplastic disorders and cancers (e.g., cancers of kidney, liver, stomach, breast, and brain) where cMET is implicated in tumor growth, angiogenesis, and metastasis. The overexpression of cMET as well as its autocrine activation by co-expression of its ligand are also implicated in oncogenesis.

Presented herein are novel anti-cMET binding agents (e.g., a monoclonal antibody) that are conjugated to a cytotoxic payload, pharmaceutical compositions thereof and methods of using the same.

### SUMMARY OF THE INVENTION

In some aspects, presented herein is a binding agent-drug conjugate comprising a binding agent and a payload, where the payload comprises a pyrrolobenzodiazepine toxin and the binding agent specifically binds to mesenchymal epithelial transition factor (cMET). In some embodiments, a payload comprises a linking group where the pyrrolobenzodiazepine toxin is covalently linked to the linking group, and the linking group is covalently linked to the binding agent. In some embodiments, a binding agent is a monoclonal antibody, or antigen binding portion thereof.

In some aspects, presented herein is a binding agent-drug conjugate comprising a binding agent and a payload, wherein the binding agent comprises: (i) two or more of a CDR-L1, a CDR-L2 and a CDR-L3 which are polypeptide sequences of a light chain complementarity determining region (CDR-L), wherein the CDR-L1 is selected from the amino acid sequences of SEQ ID NOs: 1-15, the CDR-L2 is selected from the amino acid sequences of SEQ ID NOs: 16-25, and the CDR-L3 is selected from the amino acid sequences of SEQ ID NOs: 26-36, and (ii) two or more of a CDR-H1, a CDR-H2 and a CDR-H3 which are polypeptide sequences of a heavy chain complementarity determining region (CDR-H), wherein the CDR-H1 is selected from the amino acid sequences of SEQ ID NOs: 50-61, the CDR-H2 is selected from the amino acid sequences of SEQ ID NOs: 62-78, and the CDR-H3 is selected from the amino acid sequences of SEQ ID NOs: 79-93; and the payload comprises a pyrrolobenzodiazepine toxin and a linking group; wherein the pyrrolobenzodiazepine toxin is covalently linked to the linking group, the linking group is covalently linked to the binding agent, and the binding agent specifically binds to an extracellular domain of mesenchymal epithelial transition factor (cMET).

In certain embodiments, a pyrrolobenzodiazepine toxin comprises the structure of chemical formula I:

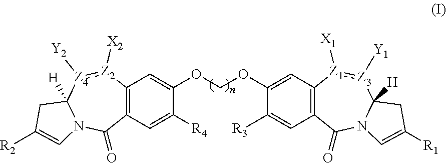

wherein
Z₁ and Z₂ are both N;
Z₃ and Z₄ are both C;
the double-dash lines [symbol] represent a single bond or a double bond;
n is 1 to 12;
each of R₃ and R₄ are independently H, or a C₁₋₄ alkoxyl; and
each of R₁ and R₂ are independently selected from the group consisting of H, C₁₋₅ alkyl, C₃₋₆ cycloalkyl, C₂₋₅ alkenyl, and a phenyl optionally substituted with R₅, wherein
   R₅ is selected from the group consisting of —NH₂, —NHR₆, and a piperazinyl substituted with R having the structure

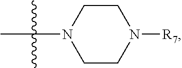

R₆ comprises the linking group, and
R₇ is H, or a C₁₋₅ alkyl;
X₁ is null, a protecting group, or comprises the linking group;
X₂ is null, a protecting group, or comprises the linking group;
only one of X₁, X₂, R₁, and R₂ comprises the linking group; and
each of Y₁ and Y₂ are independently either null, OH, or SO₃H;
   provided that:
   (i) when X₁ comprises the linking group, Z₁ [symbol] Z₃ is N—C.
   (ii) when X₂ comprises the linking group, Z₂ [symbol] Z₄ is N—C.,
   (iii) when X₁ comprises the protecting group, Z₁ [symbol] Z₃ is N—C, and
   (iv) when X₂ comprises the protecting group, Z₂ [symbol] Z₄ is N—C.
      wherein null indicates the absence of the moiety or the presence of one or more hydrogens to complete a required valency.

In some embodiments of the pyrrolobenzodiazepine toxin of the structure of chemical formula I, n is 3 or 5. In some embodiments of the pyrrolobenzodiazepine toxin of the structure of chemical formula I, R₃ and R₄ are both —O—CH₃. In certain embodiments of the pyrrolobenzodiazepine toxin of the structure of chemical formula I, R₁ and R₂ are both methyl or R₁ and R₂ are both —CH═CH—CH₃. In some embodiment of the pyrrolobenzodiazepine toxin of the structure of chemical formula I, R₂ is a cyclopropyl. In certain embodiments of the pyrrolobenzodiazepine toxin of the structure of chemical formula I, R₂ is phenyl substituted with 4-methylpiperazin-1-yl or phenyl substituted with R₅, where R₅ is —NHR₆ and R₆ comprises the linking group. In certain embodiments of the pyrrolobenzodiazepine toxin of the structure of chemical formula I, the linking group is attached to the pyrrolobenzodiazepine toxin by a carbamate group or an amide group.

In some embodiments of the pyrrolobenzodiazepine toxin of the structure of chemical formula I, X₁ is pull, Y₁ is null, Z₁ [symbol] Z₃ is N═C, X₂ is pull, Y₂ is null and Z₂ [symbol] Z₄ is N═C. In certain embodiments of the pyrrolobenzodiazepine toxin of the structure of chemical formula 1, X₁ comprises the linking group, Y₁ is a OH, Z₂ [symbol] Z₄ is N═C, X₂ is null, and Y₂ is null. In certain embodiments of the pyrrolobenzodiazepine toxin of the structure of chemical formula I, X₁ comprises the linking group, Y₁ is OH, Z₂ [symbol] Z₄ is N—C, X₂ is a protecting group, and Y₂ is OH.

In certain embodiments, a linking group comprises the structure of chemical formula A:

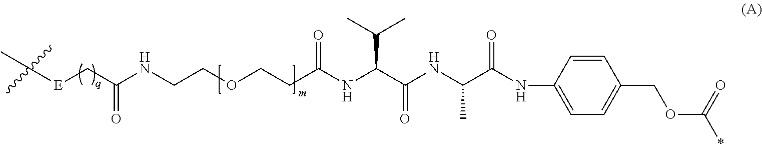

wherein the asterisk indicates the point of attachment to a pyrrolobenzodiazepine toxin; the wavy line indicates the point of attachment to the binding agent; m is 1 to 20; q is 1 to 10; and E is a connecting group. In certain embodiments of the linking group of chemical formula A, m is 4 or 8 and q is 0, 1 or 2. In some embodiments, m is 8 and q is 2.

In certain embodiments, a linking group comprises the structure of chemical formula B:

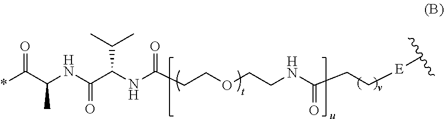

wherein the asterisk indicates the point of attachment to the pyrrolobenzodiazepine toxin; the wavy line indicates the point of attachment to the binding agent; E comprises a connecting group; v is 0 to 10; and u is 0 or 1; wherein when u is 1, tis 1 to 10. In certain embodiments of the linking group of chemical formula B, vis 1. In certain embodiments of the linking group of chemical formula B, u is 1, and tis 8. In certain embodiments of the linking group of chemical formula B, u is 0, and v is 4.

In certain embodiments of the linking group of chemical formulas A and B, a binding agent is connected to E by a thioether bond formed between a cysteine thiol residue of the binding agent and E. In some embodiments, E comprises the structure of chemical formula C:

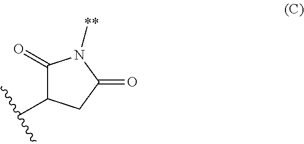

wherein the wavy line indicates the point of attachment to a binding agent and the double asterisk indicates a point of attachment to the linking group.

In some embodiments of the pyrrolobenzodiazepine toxin of the structure of chemical formula I, the protecting group has the following structure (D):

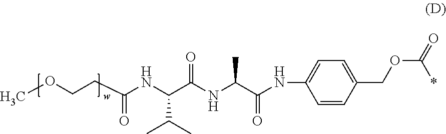

wherein the asterisk indicates a point of attachment to a pyrrolobenzodiazepine toxin; and w is 1 to 5. In some embodiments, w is 2. In some embodiments, a protecting group is a cleavable protecting group.

In certain aspects, presented herein is a binding agent-drug conjugate comprising a binding agent and a payload, where the binding agent specifically binds to an extracellular domain of mesenchymal epithelial transition factor (cMET), the payload is covalently linked to the binding agent, and the payload comprises a structure selected from the group consisting of chemical formulas II, III, V, VI and VII, where chemical formula II comprises the structure:

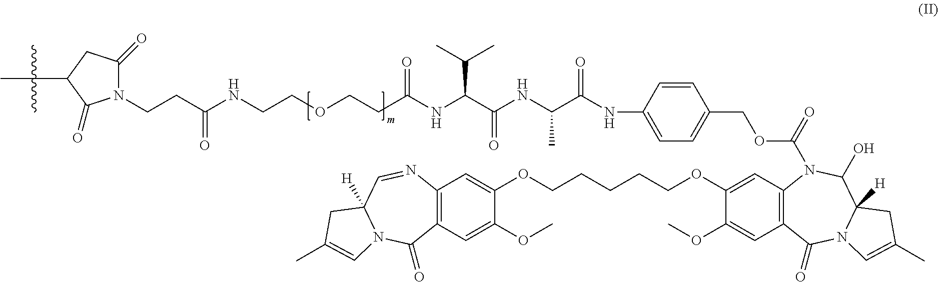

wherein m is 8 and the wavy line indicates the point of attachment to a binding agent;
chemical formula III comprises the structure:

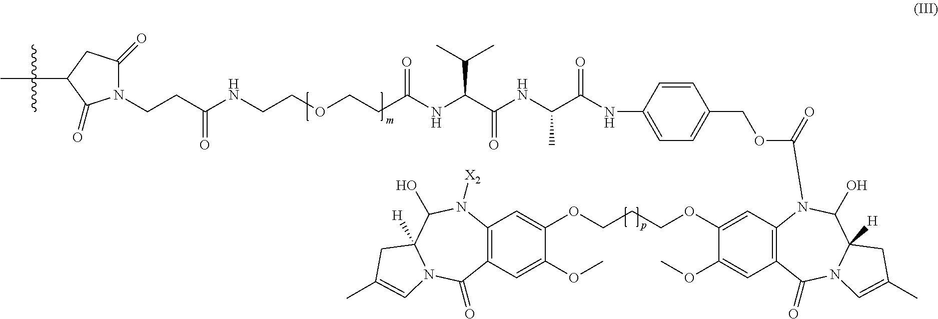

wherein m is 8, p is 2 or 3, X₂ is a protecting group and the wavy line indicates the point of attachment to a binding agent;
chemical formula V comprises the structure:

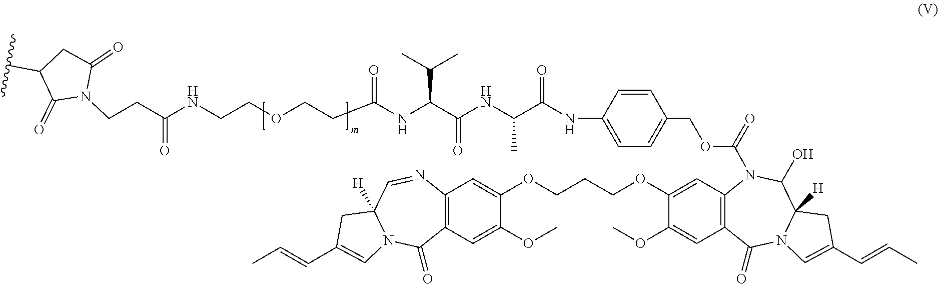

wherein m is 8, and the wavy line indicates a point of attachment to a binding agent;
chemical formula VI comprises the structure:

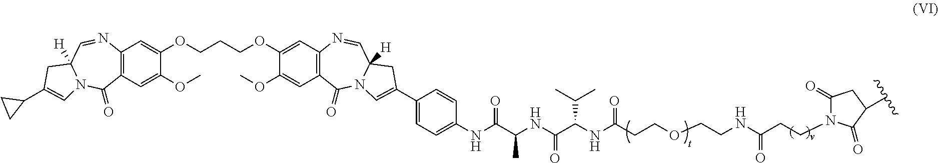

wherein t is 8, v is 1 and the wavy line indicates a point of attachment to a binding agent;
and chemical formula VII comprises the structure:

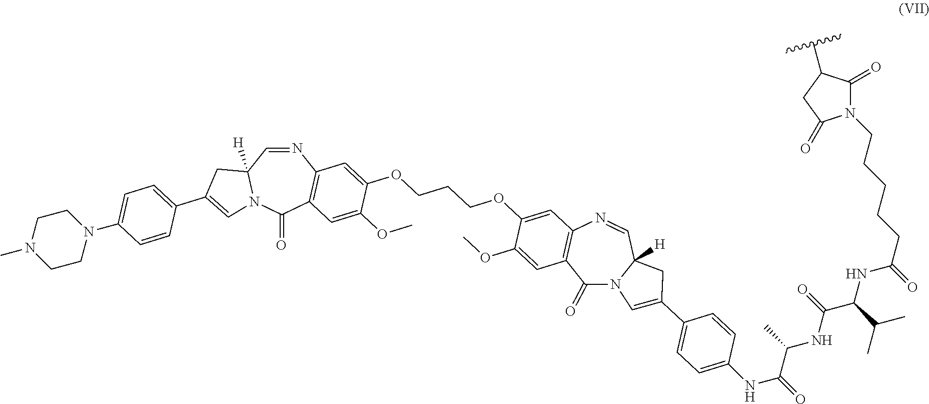 [SMILES: *C1CC(=O)N(CCCCCC(=O)N[C@H](C(=O)N[C@@H](C)C(=O)Nc2ccc(C3=CN4C(=O)c5cc(OC)c(OCCCOc6cc7c(cc6OC)C(=O)N6C=C(c8ccc(N9CCN(C)CC9)cc8)C[C@H]6C=N7)cc5N=C[C@@H]4C3)cc2)C(C)C)C1=O]

wherein the wavy line indicates the point of attachment to a binding agent.

In certain embodiments, the protecting group of X₂ has the following structure (D):

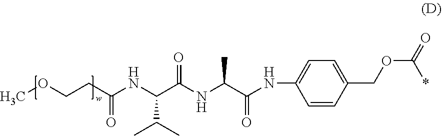

wherein the asterisk indicates the point of attachment to the payload; and w is 1 to 5.

In certain aspects presented herein is a binding agent-drug conjugate comprising a monoclonal antibody, or antigen binding portion thereof, and a payload, wherein the monoclonal antibody, or antigen binding portion thereof, comprises a CDR-L1 selected from the amino acid sequences of SEQ ID NOs: 2, 4, 6, 8, 10, 12 and 14, a CDR-L2 selected from the amino acid sequences of SEQ ID NOs: 17, 19, 21, 23 and 25, a CDR-L3 selected from the amino acid sequences of SEQ ID NOs: 27, 29, 31, 33 and 35, a CDR-H1 selected from the amino acid sequences of SEQ ID NOs: 51, 53, 55, 57 and 59, a CDR-H2 is selected from the amino acid sequences of SEQ ID NOs: 63, 65, 67, 69, 73 and 75, and a CDR-H3 selected from the amino acid sequences of SEQ ID NOs: 80, 82, 84, 86, 88, 91 and 93.

In certain embodiments, a binding agent of a binding agent-drug conjugate described herein comprises a CDR-L1 comprising the amino acid sequence of SEQ ID NO: 10 or 14; a CDR-L2 comprising the amino acid sequence of SEQ ID NO: 25; a CDR-L3 comprising the amino acid sequence of SEQ ID NO: 35; a CDR-H1 comprising the amino acid sequence of SEQ ID NO: 59; a CDR-H2 comprising the amino acid sequence of SEQ ID NO: 71; and a CDR-H3 comprising the amino acid sequence of SEQ ID NO: 88.

In certain embodiments, a binding agent of a binding agent-drug conjugate described herein comprises a CDR-L1 comprising the amino acid sequence of SEQ ID NO: 9 or 15; a CDR-L2 comprising the amino acid sequence of SEQ ID NO: 24; a CDR-L3 comprising the amino acid sequence of SEQ ID NO: 34; a CDR-H1 comprising the amino acid sequence of SEQ ID NO: 58; a CDR-H2 comprising the amino acid sequence of SEQ ID NO: 70 or 78; and a CDR-H3 comprising the amino acid sequence of SEQ ID NO: 87.

In some embodiments, a binding agent comprises a variable light chain region comprising an amino acid sequence selected from the amino acid sequences of SEQ ID NOs: 37-44. In some embodiments, a binding agent comprises a variable light chain sequence having at least 90% sequence identity, or 100% sequence identity to any one of the amino acid sequences of SEQ ID NOs: 45-49. In some embodiments, a binding agent comprises a variable light chain sequence having at least 90% sequence identity to any one or SEQ ID NOs: 37-49, wherein the variable light chain sequence has one to ten, or one to five amino acid modifications selected from an amino acid addition, an amino acid deletion and an amino acid substitution.

In some embodiments, a binding agent comprises a variable heavy chain region comprising an amino acid sequence selected from the amino acid sequences of SEQ ID NOs: 94-103. In some embodiments, a binding agent comprises a variable heavy chain region having at least 90% sequence identity, or 100% sequence identity to any one of the amino acid sequences of SEQ ID NOs: 104-108. In some embodiments, a binding agent comprises a variable heavy chain region having at least 90% sequence identity to any one of the amino acid sequences of SEQ ID NOs: 94-108, wherein the variable heavy chain sequence has one to ten, or one to five amino acid modifications selected from an amino acid addition, an amino acid deletion and an amino acid substitution.

In certain embodiments, a binding agent comprises a variable light chain sequence having at least 90% sequence identity to any one or SEQ ID NOs: 37-49, and a variable heavy chain sequence having at least 90% sequence identity to any one of the amino acid sequences of SEQ ID NOs: 94-108.

In certain embodiments, a binding agent, monoclonal antibody, or antigen binding portion thereof, is chimeric or humanized. For example, in some embodiments, a binding agent comprises one or more humanized or human framework region and/or one or more mouse framework regions.

In certain embodiments, a binding agent, monoclonal antibody, or antigen binding portion thereof comprises: a heavy chain and light chain each having CDRs that are the same as respective CDRs of heavy chain and light chain of an antibody produced by hybridoma cell line F6B1P3D12 deposited with ATCC on Mar. 20, 2019 under ATCC PTA-125826.

A binding agent-drug conjugate comprising a binding agent and a payload, wherein the binding agent comprises: a heavy chain and light chain each having CDRs that are the same as respective CDRs of heavy chain and light chain of an antibody produced by hybridoma cell line F6B1P3D12 deposited with ATCC on Mar. 20, 2019 under ATCC PTA-125826, and the payload comprises a pyrrolobenzodiazepine toxin and a linking group; wherein the pyrrolobenzodiazepine toxin is covalently linked to the linking group, the linking group is covalently linked to the binding agent, and the binding agent specifically binds to an extracellular domain of mesenchymal epithelial factor (cMET).

In some embodiments, a binding agent that specifically binds to cMET, or a portion thereof, comprises an antigen binding portion of an antibody or a single chain antibody. For example, in certain embodiments, a binding agent of a binding agent-drug conjugate comprises a Fab, Fab′, F(ab′)2, Fv or scFV fragment of an antibody.

In certain embodiments, a binding agent binds specifically to a mammalian cMET. In certain embodiments, a binding agent binds specifically to a human cMET, monkey cMET and/or rat cMET. In some embodiments, a binding agent specifically binds to the extracellular domain of a wild type or variant cMET. In some embodiments, a binding agent that specifically binds to cMET is a binding agent that induces internalization and/or degradation of cMET on a human cancer cell. In some embodiments, a binding agent that specifically binds to cMET is a binding agent that is not a cMET agonist. Accordingly, a cMET binding agent that is not a cMET agonist is a binding agent, that upon binding to a cell-surface cMET, does not substantially induce signaling through the cell-surface cMET.

In certain aspects, presented herein is a pharmaceutical composition comprising the binding agent-drug conjugate described herein and a pharmaceutically acceptable excipient, diluent, additive or carrier.

In certain aspects, presented herein is a method of treating a subject having a neoplastic disorder or cancer, the method comprising administering a therapeutically effective amount of the binding agent-drug conjugate described herein to a subject having, or suspected of having, a neoplastic disorder or cancer. In certain embodiments, a binding agent-drug conjugate blocks, inhibits, ameliorates, abrogates, or suppresses growth, viability or metastasis of the cancer. In certain embodiments, a binding agent-drug conjugate induces death, necrosis or apoptosis of some or all of the cancer. In certain embodiments, a neoplastic disorder or cancer comprises a carcinoma, sarcoma, neuroblastoma, glioblastoma, myeloma, lymphoma, melanoma or a solid or soft tissue tumor. In certain embodiments, a neoplastic disorder or cancer comprises a bladder cancer, breast cancer, colorectal cancer, gastric cancer, pancreatic cancer (e.g., exocrine pancreatic cancer and pancreatic neuroendocrine cancer), esophageal cancer, liver cancer, hepatocellular cancer, hypopharynx cancer, lung cancer, adenocarcinoma, ovarian cancer or renal cancer. In certain embodiments, a neoplastic disorder or cancer comprises a pancreatic adenocarcinoma, colorectal adenocarcinoma, small intestinal malignancy, cholangiocarcinoma, non-small cell lung cancer (NSCLC), thyroid carcinoma, esophageal or esophagogastric junction (EGJ) cancer, gastric adenocarcinoma, liver hepatocellular carcinoma, head and neck squamous carcinoma, female genital tract malignancy, breast carcinoma, lung small cell carcinoma, ovarian surface epithelial carcinoma, retroperitoneal or peritoneal sarcoma, prostatic adenocarcinoma, neuroendocrine tumor, gastrointestinal stromal tumor, glioblastoma or non-epithelial ovarian cancer. In certain embodiments, a cancer that can be treated by a method described herein is a cancer comprising a cell (e.g., a malignant or neoplastic cell) that expresses a cMET polypeptide (e.g., cMET, e.g., expressed on the cell surface).

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a summary of the work flow used for generation of monoclonal antibodies (exemplary binding agents) that bind specifically to cMET. The lead monoclonal Ab P3D12 was generated from a mouse immunized with recombinant intact extracellular domain of cMET fused to human Fc.

FIG. 2 shows an immunization scheme used to generate monoclonal antibodies (exemplary binding agents) that bind specifically to cMET. Mice were initially immunized by intraperitoneal injection (i.p.) with 100 μg of a human cMET-Fc fusion protein (cMET-Fc), or 50 to 100 μg of a KLH conjugated cMET peptide in Freund's Complete Adjuvant (CFA) as indicated. cMET-Fc comprises the extracellular domain of human cMET fused to an Fc portion of an antibody. cMET peptides were strategically selected from a portion of the cMET extracellular domain. Immunized mice received one or more booster immunization comprising 25 or 50 μg of cMET-Fc or peptide in Incomplete Freund's adjuvant (IFA) as indicated. Some mice received repetitive immunizations at multiple sites (RIMMS). Immunizations included Met-Fc fusions, peptides, traditional and RIMMS. Spleens of immunized mice were obtained and fused to a suitable fusion partner. Over 20,000 hybridoma clones were obtained and screened.

FIG. 3 shows a 3D structure of the MET SEMA domain bound with a Fab of an agonist Met-mAb antibody (5D5 Fab) and HGF beta subunit (HGFβ or HGFb). The bottom arrow indicates the position of a portion of cMET used to design peptide 3.

FIG. 4 shows characterization results from an exemplary fusion (FUSION 6B1, plate 3). Anti-cMET hybridomas were selected, in part, for the presence of specific binding to cMET as assayed by ELISA (see column labeled “MET Binding ELISA OD₄₅₀nm”) and for ability to induce internalization of cMET on human cancer cell lines as measured by flow cytometry (see column labeled “FACS Geom. Mean”). A FACS Geom. Mean value lower than a negative control indicates internalization of cMET. The arrow indicates a lead hybridoma F6B1P3D12.

FIG. 5 shows the results of a MET degradation assay. Anti-cMET antibodies isolated from the indicated wells (x-axis) were tested and selected for their ability to induce degradation of cMET on human cancer cell lines as measured by Mesoscale (MSD) cMET protein quantification. Relative values of Met degradation are indicated on the y-axis as % control (percent of control). Values lower than 100% control (negative control level) indicate internalization and degradation of cMET. Degradation indicates not just internalization but lysosomal trafficking, an important property for an antibody drug conjugate. The arrow indicates the results for a lead hybridoma F6B1P3D12.

FIG. 6 shows the results of a phospho-ERK assay (P-ERK assay) that measures agonist activity of anti-cMET antibodies by indirectly measuring the phosphorylation of ERK induced by binding of an anti-cMET antibody to cMET on a cell surface. The amount of phosphorylated ERK (shown as % of control, y-axis) detected in cell lysates after treatment of viable cells with an anti-cMET antibody is shown. Anti-cMET antibodies produced from various anti-cMET hybridomas (x-axis) were tested at 6 μg/ml or 30 μg/ml (as indicated on x-axis), and selected according to their inability to induce significant phosphorylation of ERK (i.e., their ability not to induce proliferation, i.e., absence of agonist activity). A lead monoclonal antibody (mAb) P3D12 is indicated by the arrow.

FIG. 7 shows results of six cMET monoclonal antibodies (mAbs) that were assayed by ELISA for binding to human, monkey (Cynomolgus Macaque, “Cyno”), canine, rat, and mouse cMET. All monoclonal antibodies bound human and monkey cMET. P3D12 demonstrated significant cross-reactivity with rat cMET. Various concentrations of each antibody are indicated on the x-axis. Relative amounts bound are indicated on the y-axis (OD450 nm).

FIG. 8A shows an alignment of the amino acid sequences of the light chain variable regions of nine mouse monoclonal anti-cMET antibodies, the names of which are indicated to the left of each sequence. SEQ ID NOs: are indicated to the right of each sequence. Amino acid sequences of the light chain variable regions of LC F6B1P1E2 and F6BP3E2 are 100% identical. Also, amino acid sequences of the light chain variable regions of LC F6B1P3D12 and F6B1P3E9 are 100% identical.

FIG. 8B shows an alignment of the amino acid sequences of the heavy chain variable regions of nine mouse monoclonal anti-cMET antibodies, the names of which are indicated to the left of each sequence. SEQ ID NOs: are indicated to the right of each sequence. Amino acid sequences of the heavy chain variable regions of F6B1P3D12H7913 and F6B1P3E9 are 100% identical. Also, amino acid sequences of the heavy chain variable regions of F6B1P1E2H7819 and F6BP3E2 are 100% identical.

FIGS. 9A and 9B show the results of an in vivo xenograft mouse model evaluating the efficacy of the indicated anti-cMET antibody drug conjugates (ADC) using the MKN45 tumor model (a cMET+gastric cancer model) in nude mice. Animals were treated once with ADCs at 2.5 mg/kg (9A) or 5.0 mg/kg (9B). The efficacy of each drug conjugated anti-cMET binding agent is compared to PBS or an unrelated non-targeting monoclonal antibody (IgG-ADC). Tumor volume (y-axis) was measured at various time points after inoculation (y-axis, Days (post inoculation)). Inhibition of tumor growth indicates positive efficacy. Anti-cMET binding agents and the non-targeting control monoclonal antibody (IgG) were conjugated to monomethyl auristatin F (MMAF).

FIG. 10 shows the results of a phospho-ERK assay (P-ERK assay) that measures agonist activity of anti-cMET antibodies by indirectly measuring the phosphorylation of ERK induced by binding of an anti-cMET antibody to cMET on a cell surface. The amount of phosphorylated ERK (shown as % of control, y-axis) detected in cell lysates after treatment of viable cells with an anti-cMET antibody is shown. The constant regions of the heavy and light chains of an isolated mouse monoclonal antibody designated as P3D12 were replaced with antibody constant regions of a human IgG1 (P3D12 (hIgG1)), or a human IgG2 (P3D12 (hIgG2)) as indicated on the x-axis. Each antibody was tested at 0.00064 μg/ml, 0.0032 μg/ml, 0.016 μg/ml, 0.08 μg/ml, 0.4 μg/ml, 2 μg/ml, 10 μg/ml and 50 μg/ml as indicated on the x-axis. “Control” indicates an untreated negative control. HGF (EC90) is a positive control and indicates cells treated with Hepatocyte Growth Factor (HGF), the natural ligand of cMET receptor. This data indicates that the human IgG2 isotype does not display detectable agonist activity.

FIG. 11 shows the results of a MET degradation assay. Degradation is a measure of internalization of the cMET receptor upon antibody binding. Chimeric anti-cMET antibodies were tested for their ability to induce degradation of cMET on human cancer cell lines as measured by Mesoscale (MSD) cMET protein quantification. Relative values of Met degradation are indicated on the y-axis as % control (percent of control). Values lower than 100% control indicate internalization and degradation of cMET. Chimeric antibodies were generated by replacing the constant regions of the heavy and light chains of an isolated mouse monoclonal antibody designated as P3D12 (P3D12 (mouse)) with antibody constant regions of a human IgG1 (P3D12 (hIgG1)), or a human IgG2 (P3D12 (hIgG2)) as indicated on the x-axis. Each antibody was tested at 0.00064 μg/ml, 0.0032 μg/ml, 0.016 μg/ml, 0.08 μg/ml, 0.4 μg/ml, 2 μg/ml, 10 μg/ml and 50 μg/ml as indicated on the x-axis. Chimeric cMET antibodies of P3D12 showed similar internalization/degradation activity as the parent mouse P3D12 antibody.

FIG. 12 shows a flow chart of process development for testing and selection of lead anti-cMET monoclonal binding agents.

FIG. 13 shows an alignment of five humanized light chain variable regions of humanized versions of the murine anti-cMET clone P3D12, the names and SEQ ID NOs: of which are indicated to the left of each sequence. Five independent methods were used to humanize the murine anti-cMET mAbs. Two of the methods gave the same result, therefore there are four different light chains shown.

FIG. 14 shows an alignment of five humanized heavy chain variable regions of humanized versions of the murine clone P3D12, the names and SEQ ID NOs: of which are indicated to the left of each sequence. Five independent methods were used to humanize the murine anti-cMET mAb. Two of the methods gave the same result, therefore there are four different light chains (see FIG. 13) and four different heavy chains which can make a combination of 16 different binding agents.

FIG. 15 shows the results of an in vivo xenograft mouse model testing the efficacy of the indicated humanized anti-cMET antibody drug conjugates (ADC) at 2.5 mg/kg (2.5 mpk), or at 5 mg/kg (5 mpk) as demonstrated using an MKN45 tumor model (a cMET+gastric cancer model). Animals were treated once with the indicated ADCs at 2.5 or 5.0 mg/kg. The efficacy of each anti-cMET binding agent is compared to PBS or the non-targeting monoclonal antibody Rituximab (Retux), which is an anti-cancer monoclonal antibody that targets CD20, which is primarily found on the surface of immune system B cells. Tumor volume (y-axis) was measured at various time points (y-axis) after inoculation. Inhibition of tumor growth indicates positive efficacy. Anti-cMET binding agents were conjugated to monomethyl auristatin F (MMAF).

FIG. 16 shows binding of anti-cMET monoclonal binding agent hD12 comprising heavy and light chain variable regions of SEQ ID NO:108 and 47, respectively, and a human IgG2 constant regions, and a negative control antibody (Denosumab), that does not bind to cMET-Fc (hMET-Fc) or mutant cMET (E168D) Fc recombinant fusion proteins. The E168D mutation is a somatic mutation found in small cell lung cancer (SCLC). The mutation is located in the Sema domain and leads to constitutive activation of the cMET receptor. Abundance of somatic mutations of cMET are very low. E168D occurs in 0.8% to 3% of SCLC patients. A binding ELISA was performed with human cMET or E168D cMET extracellular domains fused to human IgG1 Fc. cMET proteins were coated on a plate overnight and the samples titrated and detected with a goat anti-human IgG (H+L)-HRP. EC₅₀s were determined using sigmoidal dose response fit.

FIG. 17A shows the results of an ELISA-based cMET binding assay. Briefly, five representative antibody-drug conjugates (i.e., hD12-VI, hD12-II, hD12-VII, hD12-IV and hD12-XI (see antibody nomenclature explained in Example 3)) were tested at increasing concentrations (x-axis, concentration of antibody-drug conjugate (μg/ml)) for their ability to bind plate-bound human cMET. Relative binding strength is indicated by luminescence (y-axis). A negative control antibody (Denosumab), that does not bind cMET, was conjugated to each of the five different payloads (i.e., payloads of chemical formula VI, II, IV, VII and XI, indicated as Denosumab-VI, Denosumab-II, Denosumab-IV, Denosumab-VII, and Denosumab-XI, respectively) and tested as a negative control. FIG. 17B shows IC₅₀ values for each of the antibodies tested in FIG. 17A.

FIG. 18A-18D show the results of a cytotoxicity assay for five representative antibody-drug conjugates (hD12-VI, hD12-II, hD12-VII, hD12-IV and hD12-XI) on four cell lines that express different amounts of cMET on their cell surface (SNU-1, no expression of cMET (FIG. 18A), SNU-16, medium expression of cMET (FIG. 18B), SNU-620, high expression of cMET (FIG. 18C) and MKN-45, high expression of cMET (FIG. 18D)). Percent survival is indicated on the y-axis and the amount of antibody drug conjugate added is indicated on the x-axis (pM). The results are summarized in FIG. 18E which shows comparative IC₅₀ values.

FIG. 19A-19E show the results of a cytotoxicity assay for five representative antibody-drug conjugates (hD12-VI, hD12-II, hD12-VII, hD12-IV and hD12-XI) on five cell lines that express different amount of cMET on their cell surface (H441, medium expression of cMET (FIG. 19A), H1373, medium expression of cMET (FIG. 19B), H1975, medium expression of cMET (FIG. 19C), SNU-5, high expression of cMET (FIG. 19D), and H1573, medium expression of cMET (FIG. 19E)). Percent survival is indicated on the y-axis and the amount of antibody drug conjugate added is indicated on the x-axis (pM). The results are summarized in FIG. 19F, which shows comparative IC₅₀ values.

FIGS. 20A-G shows the results of an in vivo xenograft study. Mice were injected with H1975 tumor cells (medium expression of cMET) and treated with either hD12-XI (FIGS. 20A, 20B and 20C), hD12-VI (FIGS. 20A, 20B and 20D), hD12-II (FIGS. 20A, 20B and 20E), hD12-VII (FIGS. 20A, 20B and 20F), and hD12-IV (FIGS. 20A, 20B and 20G), and tumor volume (y-axis, FIGS. 20A, and 20C-20G) or body weight (FIG. 20B) was determined over time (i.e., days, x-axis). A negative control antibody (Denosumab) conjugated to each of the five different payloads (i.e., VI, II, IV, VII and XI) was tested as a negative control.

FIGS. 21A-E shows the results of an in vivo xenograft study. Mice were injected with H1373 tumor cells (medium expression of cMET) and treated with either hD12-XI (FIG. 21A), hD12-VI (FIG. 21B), hD12-II (FIG. 21C), hD12-VII (FIG. 21D), and hD12-IV (FIG. 21E) and tumor volume (y-axis) was determined over time (i.e., days, x-axis). A negative control antibody (Denosumab) conjugated to II was tested as a negative control.

FIG. 22 shows the body weight (y-axis) of the mice treated in FIG. 21 over time (x-axis).

FIG. 23 shows the serum concentration (y-axis) of hD12-XI, hD12-VI, hD12-II, hD12-VII, hD12-IV, and Denosumab-II after i.v. injection in mice. Time (hours) after injection is indicated on the x-axis. The concentration of each antibody-drug conjugate was determined by ELISA.

FIGS. 24A-B shows the results of an ELISA-based cMET binding assay showing the relative affinity of hD12 and four variants of hD12 (hD12-T289C, hD12-V442C and hD12-V282C) that are covalently linked to the payload II, for plate-bound human cMET. hD12-II was stochastically linked to the payload at random sulfhydryl groups. hD12-T289C-II, hD12-V442C-II and hD12-V282C-II were site-specially linked to the payload at the position of the variant cysteine residue (i.e., T289C, V442C and V282C, respectively). The antibody drug conjugates were tested at increasing concentrations (x-axis, concentration of antibody-drug conjugate (μg/ml)) for their ability to bind plate-bound human cMET. Relative binding strength is indicated by luminescence (y-axis). A negative control antibody (Denosumab-II) was tested as a negative control. FIG. 24B shows IC₅₀ values for each of the antibodies tested in FIG. 24A.

FIGS. 25A-G shows the results of a cytotoxicity assay for the antibody conjugates of FIG. 24 (i.e., hD12-II, hD12-T289C-II, hD12-V442C-II and hD12-V282C-II) on four cell lines that express different amounts of cMET on their cell surface (SNU-1, no expression of cMET (FIG. 25A), SNU-16, medium expression of cMET (FIG. 25B), SNU-620, high expression of cMET (FIG. 25C), MKN-45, high expression of cMET (FIG. 25D), and N87, low expression of cMET (FIG. 25E)). Percent survival is indicated on the y-axis and the amount of antibody drug conjugate added is indicated on the x-axis (pM). The results are summarized in FIG. 25F which shows comparative IC₅₀ values. The concentrations of antibody drug conjugate tested are indicated in FIG. 25G.

FIG. 26A shows the results of a cytotoxicity assay for the antibody conjugates of FIG. 24 (i.e., hD12-II, hD12-T289C-II, hD12-V442C-II and hD12-V282C-II) on a high cMET expressing cell line (i.e., SNU-5). Percent survival is indicated on the y-axis and the amount of antibody drug conjugate added is indicated on the x-axis (pM). The results are summarized in FIG. 26B which shows comparative IC₅₀ values. The concentrations of antibody drug conjugate tested are indicated in FIG. 26C.

FIGS. 27A-E shows the results of a cytotoxicity assay for the antibody conjugates of FIG. 24 (i.e., hD12-II, hD12-T289C-II, hD12-V442C-II and hD12-V282C-II) on three cell lines expressing medium amounts of cMET on their cell surface (H1373, FIG. 27A, H1573, FIG. 27B, and H1975, FIG. 27C). Percent survival is indicated on the y-axis and the amount of antibody drug conjugate added is indicated on the x-axis (pM). The concentrations of antibody drug conjugate tested are indicated in FIG. 27D. The results are summarized in FIG. 27E which shows comparative IC₅₀ values.

FIGS. 28A-C shows the results of an in vivo xenograft study. Mice were injected with H1975 tumor cells (medium expression of cMET) and treated with hD12-V442C-II (FIG. 28A), hD12-T289C-II (FIG. 28B) and hD12-V282C-II (FIG. 28C) at the indicated concentrations. Mice were treated with stochastically conjugated hD12-II as a positive control. Mice were treated with vehicle (PBS) or Denosumab-II as negative controls. Tumor volume (y-axis) was determined over time (i.e., days, x-axis).

FIG. 29 shows the body weight (y-axis) of the mice treated in FIG. 28 over time (x-axis).

FIG. 30 shows the serum concentration (y-axis) of hD12-V442C-II (FIG. 28A), hD12-T289C-II (FIG. 28B) and hD12-V282C-II (FIG. 28C) after i.v. injection in rats. Time (hours) after injection is indicated on the x-axis. The concentration of each antibody-drug conjugate was determined by ELISA.

FIG. 31 shows a table summarizing the pharmacokinetic data obtained from the experiment of FIG. 30.

FIGS. 32A-C shows the results of an in vivo patient-derived xenograft (PDX) study using human primary gastric cancer tissue (FIG. 32A), human primary colorectal cancer tissue (FIG. 32B) and human head & neck cancer tissue (FIG. 32C). Percentage of tumor growth inhibition (TGI %) is shown on the y-axis and the concentration of the antibody conjugate (hD12-T289C-II) administered is shown on the x-axis. TGI % was calculated as described in Example 12.

FIG. 33A shows immunohistochemical staining of a cross-section of gastric cancer PDX model GA3121 for human cMET protein. FIG. 33B shows an amplified view of the area shown in the white inset of FIG. 33A. FIG. 33C shows the results of tumor growth inhibition of an in vivo patient-derived xenograft (PDX) model GA3121. Each data point represents 1 group consisting of 10 mice. Mice were treated with vehicle (PBS) or sekukinumab-II as negative controls. Tumor volume (y-axis) was determined over time (i.e., days, x-axis).

### DETAILED DESCRIPTION

Presented herein, in some embodiments, are binding agent-drug conjugates comprising a binding agent (e.g., a monoclonal antibody, or antigen binding portion thereof) and a payload (e.g., a cytotoxic payload). In some embodiments, a binding agent is a novel monoclonal antibody, or an antigen binding portion thereof, that binds specifically to cMET. In some embodiments, a payload comprises a pyrrolobenzodiazepine (PBD) toxin and a specific linking group. The novel binding agent-drug conjugates presented herein are useful for treating cancer and/or neoplastic disorders.

The binding agents disclosed herein are novel, not only in the amino acid sequences of their antigen binding regions (e.g., heavy and light chain variable regions), but also in their functional characteristics. For example, the anti-cMET binding agents described herein possess a combination of distinct characteristics not found in other cMET antibodies. First, the anti-cMET binding agents described herein do not induce significant signaling (e.g., receptor tyrosine kinase activity) from a cMET receptor upon binding. Accordingly, upon binding, the cMET binding agents described herein do not induce undesirable oncogenic activity upon binding (e.g., growth, proliferation, metastasis or angiogenesis). Second, anti-cMET binding agents described herein can induce cMET degradation and are internalized after binding. The benefit to this characteristic is that any toxic payload attached to the anti-cMET binding agent is brought into the interior of a target cell thereby reducing off-target, non-specific toxicity of the payload. This feature also allows for the activity of a toxic payload to be controlled or regulated. For example, in some embodiments, a toxic payload described herein is substantially inactive until contacted with an intracellular protease. Third, certain anti-cMET binding agents described herein cross-react with non-human primates, rat and/or mouse which allows for the antibody drug conjugates to be tested and optimized using non-human animal models. Four, the anti-cMET binding agents described herein are soluble, display pro-longed half-life in vivo and are stable upon storage.

A number of cytotoxic payloads are known which can be attached to an antibody using a known linker to generate an antibody drug conjugate (ADC) (e.g., see US 2014/0120118, US 2014/0127239, US 2016/0250344, US 2016/0250345 and Tiberghien, et al., (2016) ACS Medicinal Chemistry Letters 7 (11): 983-987). However, the biochemical functionalities of an antibody binding agent often changes after conjugation to a payload. Similarly, the biochemical characteristics of a payload after conjugation to an antibody are not always predictable. For example, the in vivo activity of a known payload may differ significantly, ranging from lethal to no therapeutic effect, depending on the type of linker used and where the linker is attached to the antibody. Therefore, generating an antibody drug conjugate by combining a specific cMET binding agent with an ideal linker, a selected toxic payload and an optimal conjugation site of linker to antibody to provide for optimal delivery of the toxin, while substantially limiting adverse events (e.g., off-target toxicity), and maintaining the desirable bio-functional properties of the binding agent, is extremely challenging, time consuming and requires a substantial inventive effort. The ADCs presented herein provide for a unique combination of toxins, linkers and novel cMET binding agents that result in highly effective ADCs that provide for optimal therapeutic effects while substantially reducing or eliminating off-target toxicity.

cMET

MET is used synonymously herein with the term “cMET”. cMET is also known as hepatocyte growth factor receptor (HGFR). Human cMET (e.g., SEQ ID NO:109) comprises an immature polypeptide sequence of 1390 amino acids and includes an N-terminal single sequence from amino acids 1-24, an extracellular domain of human cMET from about amino acid 24-932, a transmembrane domain from about amino acid 933 to 955 and a cytoplasmic domain at about amino acid 956 to 1390, numbered from the N-terminus to the C-terminus. Methods of identifying leader sequences, extracellular domains, transmembrane domains, and cytoplasmic domains of a cMET receptor are known and any suitable method can be used to identify such domains or regions within a cMET polypeptide sequence derived from a suitable mammalian species. A human cMET polypeptide may comprise several known variants (e.g., see www <dot> uniprot <dot> org/uniprot/P08581, as accessed on May 5, 2016, which cMET variants and alternative sequences disclosed therein are incorporated herein by reference). Non-limiting examples of naturally occurring variants of a human cMET include amino acid substitutions at 143, 150, 156, 168, 238, 316, 320, 375, 385, 773, 970, 991, and/or 992 of human cMET (SEQ ID NO: 109). In some embodiments cMET or a cMET extracellular domain comprises an E to D substitution at position 168 of human cMET, referred to herein as E168D. In some embodiments cMET or a cMET extracellular domain comprises an N to S substitution at position 375 of human cMET, referred to herein as N375S.

In some embodiments cMET is a mammalian cMET. In some embodiments cMET is a primate cMET. In some embodiments cMET is a human cMET. In some embodiments cMET is a monkey cMET. In some embodiments cMET is a rodent cMET (e.g., rat and/or mouse). In some embodiments cMET is a canine cMET (e.g., a dog cMET). Non-limiting examples of a mammalian cMET are provided in Example 5 and/or in a sequence listing of this application. In certain embodiments, an extracellular domain of cMET comprises an N-terminal portion of a cMET polypeptide that is typically expressed on the cell surface of an intact mammalian cell. An extracellular domain of cMET may comprise two or more polypeptide chains derived from a MET translation product. In certain embodiments an extracellular domain of cMET can be expressed in a soluble and/or a non-membrane bound form that lacks a cytoplasmic and/or transmembrane domain. In certain embodiments an extracellular domain of cMET is expressed, isolated and/or purified as a fusion protein. For example, the extracellular domain of a mammalian cMET can be engineered and expressed as a fusion protein comprising an Fc portion of an immunoglobulin (e.g., cMET-Fc). In certain embodiments cMET and/or the extracellular domain of cMET comprises one or more amino acid additions, deletions or substitutions. A cMET polypeptide may be at least 80%, at least 85%, at least 90% or at least 95% to a cMET polypeptide disclosed herein. In certain embodiments, a cMET polypeptide comprises a portion of (e.g., a sub-sequence of) a cMET protein. In some embodiments a portion of a cMET comprises an extracellular domain of cMET, or a portion thereof.

### Binding Agents

In certain embodiments, a binding agent comprises or consists of one or more polypeptides or one or more proteins that bind specifically to cMET or a portion thereof. In some embodiments, a binding agent comprises or consists of a protein that binds specifically to cMET or a portion thereof. A binding agent often comprises at least one antigen binding portion (i.e. a binding portion). An antigen binding portion of a binding agent is that portion that binds specifically to an antigen. In certain embodiments a binding portion of a binding agent comprises or consists of a single polypeptide (e.g., single chain antibody). In some embodiments a binding portion of a binding agent comprises or consists of two polypeptides. In some embodiments a binding portion of a binding agent comprises or consists of 2, 3, 4 or more polypeptides. In some embodiments a binding agent comprises one or more structural portions (e.g., scaffolds, structural polypeptides, constant regions and/or framework regions). In some embodiments a binding agent, or binding portion thereof is attached to a substrate (e.g., a polymer, a non-organic material, silicon, a bead, and the like).

A binding agent may comprise one antigen binding portion or multiple antigen binding portions. For example, a binding agent that comprises one binding portion is sometimes referred to as monovalent. A binding agent that comprises two binding portions is sometimes referred as divalent. In some embodiments a binding agent comprises 1, 2, 3, 4, 5, 6, 7, 8, 9, or 10 or more binding portions. In certain embodiments, all of the binding portions of a multivalent binding agent bind to the same antigen. In certain embodiments, all of the binding portions of a multivalent binding agent comprise one or more polypeptide sequences that are at least 90%, at least 95%, at least 99% or 100% identical.

In certain embodiments, two or more binding portions of a binding agent bind to different antigens. Such binding agents are sometimes referred to as bi-specific or multi-specific binding agents (e.g., antibodies). Thus, in certain embodiments a binding agent comprises a first antigen binding portion that specifically binds cMET, or a portion thereof, and a second antigen binding portion that specifically binds another antigen (e.g., a polypeptide that is not cMET, or a portion thereof). A binding agent that specifically binds cMET, in some embodiments, is covalently or non-covalently attached to another binding agent that does not bind specifically to cMET, or a portion thereof. In certain embodiments, a binding agent that specifically binds cMET comprises a second binding agent the specifically binds to another antigen.

In some embodiments a binding agent comprises an antibody, or a portion thereof (e.g., a binding portion thereof). In certain embodiments, a binding agent comprises or consists of a suitable antibody, or antigen binding portion of an antibody. In some embodiments a binding agent is an antibody (e.g., a monoclonal antibody and/or a recombinant antibody). A binding agent or antibody can be generated, manufactured or produced by a suitable method. In some embodiments a binding agent is monoclonal. In some embodiments a binding agent is a monoclonal antibody derived from a suitable species. Certain non-limiting examples of a binding agent include monoclonal antibodies, chimeric antibodies, antibody binding fragments (e.g., an antigen binding portion of an antibody), a CDR-grafted antibody, a humanized antibody, a human antibody, or portions thereof. Human antibodies can be obtained by any suitable method. For example, human antibodies can be obtained from trans-chromosomal animals engineered to produce fully human antibodies. In certain embodiments, a binding agent is not polyclonal, is not a polyclonal antibody and the term “binding agent” does not refer to polyclonal antibodies.

In some embodiments a binding agent is derived, produced, obtained, isolated, and/or purified from a suitable species. In some embodiments a binding agent is derived, produced, obtained, isolated, and/or purified from a rabbit, goat, horse, cow, rat, mouse, fish, bird, or llama, for example. In some embodiments a binding agent is derived, produced, obtained, isolated, and/or purified from a bird (e.g., a chicken, or a bird egg). In some embodiments a binding agent is derived, produced, obtained, isolated, and/or purified from a plant (e.g., a recombinant binding agent produced by a genetically engineered plant). In some embodiments a binding agent is derived, produced, obtained, isolated, and/or purified from a suitable mammal. In certain embodiments a suitable mammal is a genetically altered mammal (e.g., a trans-chromosomal or transgenic mammal) engineered to produce antibodies comprising human heavy chains and/or human light chains or portions thereof. In some embodiments a binding agent is produced, obtained, isolated, or purified from a prokaryotic or eukaryotic cell (e.g., a recombinant binding agent produced by a genetically engineered cell). In some embodiments a binding agent is produced, obtained, isolated, or purified from a virus (e.g., a recombinant binding agent produced by a genetically engineered virus). A binding agent can be expressed, isolated from and/or purified from a suitable expression system non-limiting examples of which include a suitable bacteria, phage, insect, virus, plant or mammalian expression system. For example, a nucleic acid encoding a binding agent can be introduced into a suitable mammalian cell line that expresses and secretes the binding agent into the cell culture media.

In certain embodiments, a binding agent is not found in nature and is not naturally occurring. For example, in certain embodiments, a binding agent is generated artificially in an animal by administering an emulsified cocktail that includes a foreign recombinant antigen, a powerful adjuvant, and often a mineral oil and/or a detergent, thereby inducing an artificial immune response to the foreign recombinant antigen (e.g., cMET, cMET-Fc).

In certain embodiments, a monoclonal antibody or a monoclonal binding agent is a substantially homogeneous population of binding agents, or binding fragments thereof, where each individual binding agent in the population is substantially identical and/or binds to the same epitope, with the exception of possible variants that may arise during production of a monoclonal binding agent. In some embodiments such variants generally are absent or may be present in minor amounts. In contrast to polyclonal antibody preparations which typically include a population of different antibodies directed against different determinants (epitopes), each binding agent of a population of monoclonal binding agents often binds to a single determinant of an antigen. Monoclonal binding agents are often not contaminated by other immunoglobulins. One or more different monoclonal binding agents may be purposely added to a composition to form a mixture.

The modifier “monoclonal” is not to be construed as requiring production of a binding agent by any particular method. A monoclonal binding agent can be produced by any suitable method. For example, in certain embodiments, a monoclonal antibody is made by a hybridoma method described by Kohler et al. (1975) Nature, 256:495, or a variation thereof. In some embodiments a monoclonal binding agent is made by a suitable recombinant DNA method. For example, monoclonal antibodies can be made or altered by a method, or variation thereof, described in U.S. Pat. No. 5,225,539 and/or Daugherty et al. (1991) Nucleic Acids Research 19 (9): 2471-2476. A monoclonal binding agent can be made by screening a recombinant library using a suitable expression system (e.g., a phage display expression system), for example. In some embodiments a monoclonal binding agent is isolated from a phage library of binding agents, for example by using a technique described in Clackson et al. (1991) Nature 352:624-628 and/or Marks et al. (1991) J. Mol Biol, 222:581-597, or a variation thereof.

In certain embodiments, a binding agent comprises one or more structural or backbone portions, sometimes referred to as scaffolds. A binding agent may comprise a scaffold, non-limiting examples of which include a scaffold derived from an antibody, a Z domain of Protein A, gamma-B crystalline, ubiquitin, cystatin, Sac7d, a triple helix coiled coil, a lipocalin, an ankyrin repeat motif, an SH3 domain of Fyn, a Kunitz domain of a suitable protease inhibitor, a fibronectin domain, a nucleic acid polymer, the like, portions thereof or combinations thereof. In some embodiments a binding agent does not comprise a scaffold. In certain embodiments, a binding agent comprises one or more structural portions of a mammalian antibody.

In certain embodiments a binding agent comprises one or more constant regions (e.g., constant regions derived from an antibody, e.g., a mammalian antibody). In certain embodiments a binding agent comprises a constant region of an antibody light chain and/or a constant region of an antibody heavy chain. In a mammalian antibody at least two types of immunoglobulin light chains exist which are referred to as lambda (λ) and kappa (κ). A binding agent may comprise any suitable constant region of an antibody, or one or more portions thereof. In some embodiments a binding agent comprises a lambda light chain constant region or a portion thereof. In some embodiments a binding agent comprises a kappa light chain constant region or a portion thereof. In some embodiments a binding agent comprises a polypeptide that is at least 75%, at least 80%, at least 85%, at least 90%, at least 95% or at least 99% identical to a polypeptide sequence of a constant region, or portion thereof, of a light chain of a mammalian antibody. In some embodiments a binding agent comprises a polypeptide that is at least 75%, at least 80%, at least 85%, at least 90%, at least 95% or at least 99% identical to a polypeptide sequence of a constant region of an antibody light chain of a human antibody. In some embodiments a binding agent does not include a light chain constant region.

In certain embodiments a binding agent comprises a constant region of an antibody heavy chain. In mammals, an antibody can have at least five types/classes of Ig heavy chains denoted as IgA, IgD, IgE, IgG, and IgM, which are determined by the presence of distinct heavy chain constant regions, or portion thereof (e.g., CH1, CL, CH2, CH3 domains). A binding agent can include any suitable heavy chain constant region, or portion thereof. In some embodiments a binding agent comprises a heavy chain constant region of an IgG1, IgG2, IgG3 or IgG4, or one or more portions thereof. In some embodiments a binding agent comprises one or more heavy chain constant regions of an IgM, IgD, IgA, or IgE isotype, or a portion thereof.

Unless otherwise specified herein, numbering of amino acid residues in the constant region of an antibody is according to the EU numbering system as described in Edelman, G. M. et al., Proc. Natl. Acad. USA, 63, 78-85 (1969). PMID: 5257969.

In some embodiments a binding agent comprises a polypeptide that is at least 75%, at least 80%, at least 85%, at least 90%, at least 95%, at least 99% identical, or 100% identical to a polypeptide sequence of a constant region, or portion thereof, of a heavy chain of a mammalian antibody. In some embodiments a binding agent comprises a polypeptide that is at least 75%, at least 80%, at least 85%, at least 90%, at least 95%, at least 99% identical or 100% identical to a polypeptide sequence of a constant region of an antibody heavy chain of a human antibody. In some embodiments a binding agent comprises one or more additions, deletions and/or modification to a constant region. A binding agent is sometimes modified to change the antibody class, or isotype of a binding agent. In some embodiments a binding agent comprises one or more additions, deletions and/or modification (one or more amino acid substitutions, deletions or additions) to modify one or more functions of a binding agent, for example to abolish, enhance or decrease serum half-life, Fc receptor binding, complement binding (e.g., C1q binding), glycosylation, sialylation, cellular toxicity, antibody-dependent cell-mediated phagocytosis (ADCP), antibody dependent cellular cytotoxicity (ADCC), and the like. In some embodiments a binding agent does not include one or more portions of a heavy chain constant region or light chain constant region. In some embodiments a binding agent does not include a heavy chain constant region.

In some embodiments a binding agent comprises or consists of one or more variable regions of an antibody, or a portion thereof. In some embodiments a binding agent comprises one or more light chain variable regions, or a portion thereof. In some embodiments a binding agent comprises one or more heavy chain variable regions, or a portion thereof. In certain embodiments a binding agent comprises at least one light chain variable region and at least one heavy chain variable region. A light chain variable region and heavy chain variable region can be on the same or different polypeptides. In certain embodiments, an antigen binding portion of a binding agent consists of one or more heavy chain variable regions. In certain embodiments, an antigen binding portion of a binding agent consists of one or more light chain variable regions. In certain embodiments, an antigen binding portion of a binding agent consists of one or more light chain variable regions and one or more heavy chain variable regions.

In some embodiments a binding agent comprises or consists of a Fab, Fab′, F(ab′)2, Fv fragment, single chain Fv (scFv), diabody (Dab), synbody, the like and/or a combination or portion thereof. In some embodiments a binding agent is a Fab, Fab′, F(ab′)2, Fv fragment, single chain Fv (scFv), diabody (Dab), synbody, the like and/or a combination, or portion thereof (see, e.g., U.S. Pat. Nos. 6,099,842 and 5,990,296). In some embodiments a binding agent comprises a single chain polypeptide comprising one or more antigen binding portions. For example, a single chain binding agent can be constructed by joining a heavy chain variable region, or antigen binding portion thereof, with a light chain variable region, or antigen binding portion thereof, with a linker (e.g., an amino acid, a polypeptide linker) using recombinant molecular biology processes. Such single chain binding agents often exhibit specificities and affinities for an antigen similar to a parent two-chain monoclonal binding agent. Binding agents often comprise engineered regions such as CDR-grafted or humanized portions. In certain embodiments a binding agent is an intact two-chain immunoglobulin, and in other embodiments a binding agent is a Fab monomer or a Fab dimer.

Nucleic acids, or portions thereof, that encode a polypeptide of a binding agent may be cloned, subcloned, rearranged or modified for recombinant expression by a suitable cloning procedure and subsequently expressed using a suitable expression system by a method known to those skilled in the art (e.g., see Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Methods in molecular biology, edited by Benny K. C. Lo, Springer Science & Business Media, 2004; Antibody Engineering, Vol. 1, Roland E. Kontermann, Stefan Dübel, Edition 2, Publisher Springer Science & Business Media, 2010; Antibody Phage Display: Methods and Protocols, Biomed Protocols, Vol. 178 of Methods in molecular biology, Editors Philippa M. O'Brien, Robert Aitken, Springer Science & Business Media, 2004).

In mammals, the heavy chain variable region and light chain variable region of an antibody each contribute three CDRs (complementarity-determining regions) commonly referred to as CDR1, CDR2 and CDR3, that are separated and/or flanked by framework regions (e.g., FR1, FR2, FR3 and FR4). The term “CDR” as used herein refers to an amino acid sequence of a polypeptide identified as a complementarity-determining region. In certain embodiments, definitive delineation of a CDR polypeptide sequence and identification of residues comprising the binding site of a binding agent is accomplished by solving the structure of a binding agent and/or solving the structure of a binding agent-antigen complex. In certain embodiments, this can be accomplished by any suitable method, such as X-ray crystallography and/or computer modeling. In certain embodiments, various methods of analysis can be employed to identify or approximate the CDR sequences of a binding agent or antibody. For example, the amino acid sequence and/or location of CDRs in a polypeptide sequence of a binding agent, an antibody, a binding portion thereof or variable region thereof, can be identified using a suitable method, non-limiting examples of which include the Kabat system (e.g., see Kabat, E. A., et al. (1991) Sequences of Proteins of Immunological Interest, Fifth Edition, NIH Publication No. 91-3242, as well as Johnson, G. and Wu, T. T. (2000) Nucleic Acids Research 28 (1): 214-8 and/or the Chothia Numbering Scheme (e.g., Chothia & Lesk, (1987) J. Mol. Biol, 196:901-917; Chothia et al. (1989) Nature 342:878-883; and Al-Lazikani et al. (1997) JMB 273,927-948). In some embodiments the amino sequence and/or location of CDRs of an antibody can be identified using the AbM method and/or contact method. The “AbM” definition uses an integrated suite of computer programs produced by Oxford Molecular Group that model antibody structure (see e.g., Martin et al. (1989) Proc. Natl. Acad. Sci. (USA) 86:9268-9272; “AbM™, A Computer Program for Modeling Variable Regions of Antibodies,” Oxford, UK; Oxford Molecular, Ltd.). The AbM definition models the tertiary structure of an antibody from primary sequence using a combination of knowledge databases and ab initio methods, such as those described by Samudrala et al. (1999) Proteins, Structure, Function and Genetics, Suppl, 3:194-198 and Xia Y, et al. (2000) J Mol Biol. 300 (1): 171-85. In certain embodiments, a contact definition is based on an analysis of the available complex crystal structures (see e.g., MacCallum et al. (1996) J. Mol. Biol 5:732-45).

In some embodiments a binding agent and/or an antigen binding portion of a binding agent comprises at least 2, at least 3, at least 4, at least 5 or at least 6 CDRs. In some embodiments a binding agent comprises 3 to 60 CDRs (e.g., for binding agents having multiple antigen binding portions). In some embodiments a binding agent comprises 3 to 12 CDRs. In some embodiments an antigen binding portion of a binding agent comprises 1 to 6 CDR polypeptide sequences.

In certain embodiments, a binding agent and/or an antigen binding portion of a binding agent comprises one, two or three CDRs of a light chain variable region. In some embodiments a light chain variable region of a binding agent comprises one or more CDRs (e.g., one, two, three, or more CDRs). The amino acid sequences representing a CDR in a light chain variable region of an antibody or binding agent is referred to as CDR-L1, CDR-L2, and CDR-L3 which are numbered sequentially (i.e., L1, L2 and L3) in the direction from the amino terminus (N-terminus) to the carboxy terminus (C-terminus) of a light chain variable region. For example, in a polypeptide representing a light chain variable region of a binding agent, CDR-L1, when present, is the most N-terminal light chain CDR; CDR-L3, when present, is the most C-terminal light chain CDR; and CDR-L2, when present, is located (i) between CDR-L1 and CDR-L3, (ii) on the N-terminal side of CDR-L3 or (iii) on the C-terminal side of CDR-L1, of a light chain variable region or binding portion of a binding agent. The terms “CDR-L1”, “CDR-L2” and “CDR-L3” refer to, in part, an amino acid sequence of a polypeptide identified as, or disclosed herein as, a complementarity-determining region of a binding agent (e.g., a CDR of a light chain variable region). Non-limiting examples of amino acid sequences of a CDR-L1, CDR-L2 and CDR-L3 are provided in Tables 1-3, respectively. A light chain variable region or antigen binding portion of a binding agent described herein may comprise any combination of a CDR-L1, a CDR-L2, and a CDR-L3 disclosed herein, wherein the binding agent retains specific binding to cMET, or a portion thereof. In certain embodiments, a light chain variable region or antigen binding portion of a binding agent described herein comprises a single light chain CDR comprising an amino acid sequence at least 70% identical to a CDR-L3 selected from Table 3.

In certain embodiments, a light chain variable region or antigen binding portion of a binding agent described herein comprises an amino acid sequence at least 70% identical to a CDR-L3 selected from Table 3, and any other suitable CDR-L2 and/or CDR-L1 polypeptide sequence, where the binding agent retains specific binding to cMET, or a portion thereof. In certain embodiments, the light chain CDRs of a light chain variable region or antigen binding portion of a binding agent consists of a CDR-L3 and a CDR-L2, where the CDR-L3 comprises an amino acid sequence at least 70% identical to a CDR-L3 selected from Table 3 and the CDR-L2 comprises an amino acid sequence at least 70% identical to a CDR-L2 selected from Table 2. In certain embodiments, a light chain variable region or antigen binding portion of a binding agent described herein comprises an amino acid sequence at least 70% identical to a CDR-L3 selected from Table 3 and an amino acid sequence at least 70% identical to a CDR-L2 selected from Table 2, and any other suitable CDR-L1 polypeptide sequence, where the binding agent retains specific binding to cMET, or a portion thereof. In certain embodiments, a light chain variable region or antigen binding portion of a binding agent described herein comprises three light chain CDRs consisting of an amino acid sequence at least 70% identical to a CDR-L3 selected from Table 3, an amino acid sequence at least 70% identical to a CDR-L2 selected from Table 2 and an amino acid sequence selected at least 70% identical to a CDR-L1 of Table 1. In certain embodiments, a light chain variable region or antigen binding portion of a binding agent described herein comprises an amino acid sequence at least 70% identical to a CDR-L3 selected from Table 3, an amino acid sequence at least 70% identical to a CDR-L2 selected from Table 2 and an amino acid sequence at least 70% identical to a CDR-L1 selected from Table 1, where the binding agent retains specific binding to cMET, or a portion thereof.

In some embodiments a binding agent comprises one or more light chain CDRs that are at least 70%, 75%, 85%, 86%, 87%, 88%, 89%, 90%, 91%, 92%, 93%, 94%, 95%, 96%, 97%, 98%, or at least 99% identical to any one of the CDR sequences listed in Tables 1, 2 or 3. In some embodiments a binding agent or the antigen binding portion of a binding agent comprises a CDR-L1 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 1. In some embodiments a binding agent or the antigen binding portion of a binding agent comprises a CDR-L1 of any one of the sequences shown in Table 1.

TABLE 1
CDR-L1 Sequences

| ID | Clone | Amino Acid Sequence |
|---|---|---|
| SEQ ID NO: 1 | F5_P5_B9_L | RSSQTIVHGTGNTYLE |
| SEQ ID NO: 2 | F5_P5_B9_L | QTIVHGTGNTY |
| SEQ ID NO: 3 | F6A_P8_E2_L | KASENVGTYVS |
| SEQ ID NO: 4 | F6A_P8_E2_L | ENVGTY |
| SEQ ID NO: 5 | F6AP12F12_L | RSSQSLLYSINQKNYLA |
| SEQ ID NO: 6 | F6AP12F12_L | QSLLYSINQKNY |
| SEQ ID NO: 7 | F6B_P1_H5_L | RASENIYNTLA |
| SEQ ID NO: 8 | F6B_P1_H5_L | ENIYNT |
| SEQ ID NO: 9 | F6B1_P3_D12_L/ | SASSSVTSNYLY |
| | F6B1_P3_E9_L | |
| SEQ ID | F6B1_P3_D12_L/ | SSVTSNY |
| NO: 10 | F6B1_P3_E9_L | |
| SEQ ID | F6B_P2_D4_L/ | SASSSVSSNYLY |
| NO: 11 | F6B1_P1_E2_L/ | |
| | F6B_P3_E2_L | |
| SEQ ID | F6B_P2_D4_L/ | SSVSSNY |
| NO: 12 | F6B1_P1_E2_L/ | |
| | F6B_P3_E2_L | |
| SEQ ID | Consensus | SASSSV(S/T)SNYLY |
| NO: 13 | | |
| SEQ ID | P3D12 VL-abb/sdr | QSVTSNY |
| NO: 14 | | |
| SEQ ID | P3D12 VL-abb/sdr | RASQSVTSNYLY |
| NO: 15 | | |

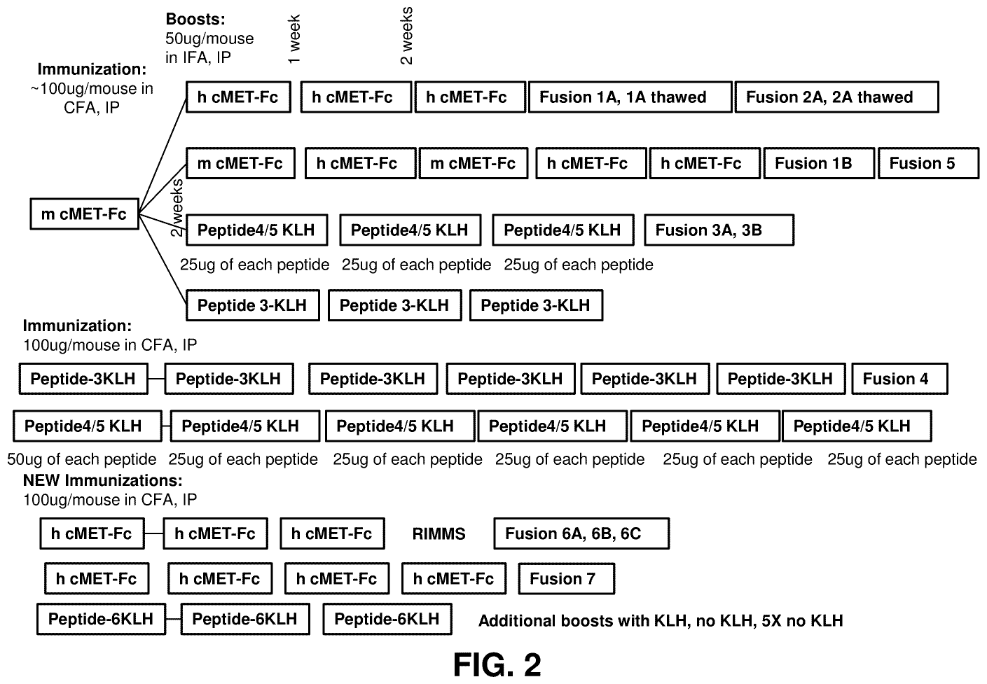

Clone names referenced in Tables 1-10 indicate the Fusion number (F), Plate number (P) and well number (A1 to H12) of a 96-well plate from which the clone was derived. Accordingly, clone F6AP12F12 was derived from Fusion 6A, Plate 12, Well F12, for example. Fusion numbers of each clone correspond to the Fusions indicated in FIG. 2.

In some embodiments a binding agent or the antigen binding portion of a binding agent comprises a CDR-L2 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 2. In some embodiments a binding agent or the antigen binding portion of a binding agent comprises a CDR-L2 of any one of the sequences shown in Table 2.

TABLE 2
CDR-L2 Sequences

| | | Amino Acid |
|---|---|---|
| ID | Clone | Sequence |
| SEQ ID NO: 16 | F5_P5_B9_L | KVSNRFS |
| SEQ ID NO: 17 | F5_P5_B9_L | KVS |
| SEQ ID NO: 18 | F6A_P8_E2_L | GASNRYT |
| SEQ ID NO: 19 | F6A_P8_E2_L | GAS |
| SEQ ID NO: 20 | F6AP12F12_L | WASTRES |
| SEQ ID NO: 21 | F6AP12F12_L | WAS |
| SEQ ID NO: 22 | F6B_P1_H5_L | AATNLAD |
| SEQ ID NO: 23 | F6B_P1_H5_L | AAT |
| SEQ ID NO: 24 | F6B1_P3_D12_L/ | STSNLAS |
| | F6B1_P3_E9_L/ | |
| | F6B_P2_D4_L/ | |
| | F6B1_P1_E2_L/ | |
| | F6B_P3_E2_L | |
| SEQ ID NO: 25 | F6B1_P3_D12_L/ | STS |
| | F6B1_P3_E9_L/ | |
| | F6B_P2_D4_L/ | |
| | F6B1_P1_E2_L/ | |
| | F6B_P3_E2_L | |

In some embodiments a binding agent or the antigen binding portion of a binding agent comprises a CDR-L3 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 3. In some embodiments a binding agent or the antigen binding portion of a binding agent comprises a CDR-L3 of any one of the sequences shown in Table 3.

TABLE 3
CDR-L3 Sequences

| ID | Clone | Amino Acid Sequence |
|---|---|---|
| SEQ ID NO: 26 | F5_P5_B9_L | FQGSHVPYTFGGGTKLEIKR |
| SEQ ID NO: 27 | F5_P5_B9_L | FQGSHVPYT |
| SEQ ID NO: 28 | F6A_P8_E2_L | GQSYSYPLTFGAGTKLELKR |
| SEQ ID NO: 29 | F6A_P8_E2_L | GQSYSYPLT |
| SEQ ID NO: 30 | F6AP12F12_L | QQYYTYPLTFGAGTKLELK |
| SEQ ID NO: 31 | F6AP12F12_L | QQYYTYPLT |
| SEQ ID NO: 32 | F6B_P1_H5_L/ | QHFWGTPYTFGGGTKLEIK |
| SEQ ID NO: 33 | F6B_P1_H5_L/ | QHFWGTPYT |
| SEQ ID NO: 34 | F6B1_P3_D12_L/ | HQWSSYPPTFGSGTKLEIK |
| | F6B1_P3_E9_L/ | |
| | F6B_P2_D4_L/ | |
| | F6B1_P1_E2_L/ | |
| | F6B_P3_E2_L | |
| SEQ ID NO: 35 | F6B1_P3_D12_L/ | HQWSSYPPT |
| | F6B1_P3_E9_L/ | |
| | F6B_P2_D4_L/ | |
| | F6B1_P1_E2_L/ | |
| | F6B_P3_E2_L | |
| SEQ ID NO: 36 | Consensus | (X₁)Q(X₂)(X₃)(X₄)YP |
| | | (X₅)T |
| | | where X₁ is H, Q, or |
| | | G; X₂ is W, S or |
| | | Y; X₃ is S or Y; X₄ is |
| | | S or T; and X₅ is P or |
| | | L. |

In some embodiments a binding agent or the antigen binding portion of a binding agent comprises a light chain variable region having at least 70%, 75%, 85%, 86%, 87%, 88%, 89%, 90%, 91%, 92%, 93%, 94%, 95%, 96%, 97%, 98%, or at least 99% identity to an amino acid sequence of Table 4. In some embodiments a binding agent or the antigen binding portion of a binding agent comprises a light chain variable region sequence of Table 4.

TABLE 4
VARIABLE LIGHT CHAIN SEQUENCES

| ID | Clone | Amino Acid Sequence |
|---|---|---|
| SEQ ID | F5_P5_B9_L | DVLMTQTPLSLPVSLGDQASISCRSSQTIVHGTGNTYLEW |
| NO: 37 | | YLQKPGQSPKLLIYKVSNRFSGVPDRFSGSGSGTDFTLKIS |
| | | RVEAEDLGVYYCFQGSHVPYTFGGGTKLEIKR |
| SEQ ID | F6A_P8_E2_L | DIVMTQSPKSMSMSVGERVTLSCKASENVGTYVSWYQQ |
| NO: 38 | | KPDQSPKLLIYGASNRYTGVPDRFTGSGSATDFTLTISSVQ |
| | | AEDLADYHCGQSYSYPLTFGAGTKLELKR |
| SEQ ID | F6AP12F12_L | DIVMSQSPSSLAVSVGEKVTMSCRSSQSLLYSINQKNYLA |
| NO: 39 | | WYQQIPGQSPKLLIYWASTRESGVPDRFTGSGSGTDFTLTI |
| | | SRVKAEDLALYYCQQYYTYPLTFGAGTKLELKR |
| SEQ ID | F6B_P1_H5_L | RCDIQMTQSPASLSVSVGETVTITCRASENIYNTLAWYLQ |
| NO: 40 | | KQGKSPQLLVYAATNLADGVPSRFSGSGSGTQYSLKINSL |
| | | QSEDFGSYYCQHFWGTPYTFGGGTKLEIKR |
| SEQ ID | F6B1_P3_D12_L | QIVLTQSPAIMSASPGEKVTLTCSASSSVTSNYLYWYQQK |
| NO: 41 | & | PGSSPKLWIYSTSNLASGVPARFSGSGSGTSYSLTISSMEA |
| | F6B1_P3_E9_L | EDAASYFCHQWSSYPPTFGSGTKLEIKR |
| SEQ ID | F6B_P2_D4_L | QIVLTQSPAIMSASPGEKVTLTCSASSSVSSNYLYWYQQK |
| NO: 42 | | PGSSPKLWIYSTSNLASGVPVRFSGSGSGTSYSLTISSMEA |
| | | EDAASYFCHQWSSYPPTFGSGTKLEIKR |
| SEQ ID | F6B1_P1_E2_L | QIVLTQSPAIMSASPGEKVTLTCSASSSVSSNYLYWYHQK |
| NO: 43 | & | PGSSPKLWIYSTSNLASGVPVRFSGSGSGTSYSLTISSMEA |
| | F6B_P3_E2_L | EDAASYFCHQWSSYPPTFGSGTKLEIKR |
| SEQ ID | Consensus | QIVLTQSPAIMSASPGEKVTLTCSASSSVSSNYLYWY(H/Q) |
| NO: 44 | | QKPGSSPKLWIYSTSNLASGVP(A/R)FSGSGSGTSYSLTISS |
| | | MEAEDAASYFCHQWSSYPPTFGSGTKLEIKR |

In some embodiments a binding agent or the antigen binding portion of a binding agent comprises a humanized light chain variable region having at least 70%, 75%, 85%, 86%, 87%, 88%, 89%, 90%, 91%, 92%, 93%, 94%, 95%, 96%, 97%, 98%, or at least 99% identity to a sequence of Table 5. In some embodiments a binding agent or the antigen binding portion of a binding agent comprises a humanized light chain variable region sequence of Table 5.

TABLE 5
Humanized P3D12 Light Chains

| ID | Clone | Amino Acid Sequence |
|---|---|---|
| SEQ ID | P3D12 VL | QIVLTQSPAIMSASPGEKVTLTCSASSSVTSN |
| NO: 45 | | YLYWYQQKPGSSPKLWIYSTSNLASGVPARFS |
| | | GSGSGTSYSLTISSMEAEDAASYFCHQWSSYP |
| | | PTFGSGTKLEIKR |
| SEQ ID | P3D12 VL- | QIVLTQSPATMSASPGERVTLSCSASSSVTSN |
| NO: 46 | ven | YLYWYQQKPGSSPRLWIYSTSNLASGVPARFS |
| | | GSGSGTSYTLTISRMEPEDAASYFCHQWSSYP |
| | | PTFGSGTKLEIKR |
| SEQ ID | P3D12 VL- | QIVLTQSPAILSLSPGERATLSCSASSSVTSN |
| NO: 47 | fra | YLYWYQQKPGSSPKLLIYSTSNLASGVPARFS |
| | | GSGSGTSYTLTISSLEAEDAASYFCHQWSSYP |
| | | PTFGSGTKLEIKR |
| SEQ ID | P3D12 VL- | QIVLTQSPATLSLSPGERATLSCRASQSVTSN |
| NO: 48 | abb/sdr | YLYWYQQKPGSSPRLLIYSTSNLASGVPARFS |
| | | GSGSGTDYTLTISRLEPEDFASYFCHQWSSYP |
| | | PTFGSGTKLEIKR |
| SEQ ID | P3D12 VL- | QIVLTQSPATLSLSPGERATLSCSASSSVTSN |
| NO: 49 | cdr | YLYWYQQKPGSSPRLLIYSTSNLASGVPARFS |
| | | GSGSGTSYTLTISRLEPEDFASYFCHQWSSYP |
| | | PTFGSGTKLEIKR |

In certain embodiments, a binding agent and/or an antigen binding portion of a binding agent comprises one, two or three CDRs of a heavy chain variable region. In some embodiments a heavy chain variable region comprises one or more CDRs (e.g., one, two, three, or more CDRs). The amino acid sequences representing a CDR in a heavy chain variable region of an antibody or binding agent is referred to as CDR-H1, CDR-H2, and CDR-H3, which are numbered sequentially (i.e., H1, H2 and H3) in the direction from the amino terminus (N-terminus) to the carboxy terminus (C-terminus) of a heavy chain variable region. For example, in a polypeptide representing a heavy chain variable region of a binding agent, CDR-H1, when present, is the most N-terminal CDR; CDR-H3, when present, is the most C-terminal CDR; and CDR-H2, when present, is located (i) between CDR-H1 and CDR-H3, (ii) on the N-terminal side of CDR-H3 or (iii) on the C-terminal side of CDR-H, of a heavy chain variable region. The terms “CDR-H1”, “CDR-H2” and “CDR-H3” refer to, in part, an amino acid sequence of a polypeptide identified as, or disclosed herein as, a complementarity-determining region of a binding agent (e.g., a CDR of a heavy chain variable region of a binding agent). Non-limiting examples of amino acid sequences of a CDR-H1, CDR-H2 and CDR-H3 are provided in Tables 6-8, respectively. A heavy chain variable region or antigen binding portion of a binding agent described herein may comprise any combination of a CDR-H1, a CDR-H2, and a CDR-H3 disclosed herein where the binding agent retains specific binding to cMET, or a portion thereof. In certain embodiments, a heavy chain variable region or antigen binding portion of a binding agent described herein comprises a single heavy chain CDR consisting of an amino acid sequence at least 70% identical to a CDR-H3 selected from Table 8. In certain embodiments, a heavy chain variable region or antigen binding portion of a binding agent described herein comprises an amino acid sequence at least 70% identical to a CDR-H3 selected from Table 8, and any other suitable CDR-H2 and/or CDR-H1 polypeptide sequence, where the binding agent retains specific binding to cMET, or a portion thereof. In certain embodiments, the heavy chain CDRs of a heavy chain variable region or antigen binding portion of a binding agent consists of a CDR-H3 and a CDR-H2, where the CDR-H3 comprises an amino acid sequence at least 70% identical to a CDR-H3 selected from Table 8 and the CDR-H2 comprises an amino acid sequence at least 70% identical to a CDR-H2 selected from Table 7. In certain embodiments, a heavy chain variable region or antigen binding portion of a binding agent described herein comprises an amino acid sequence at least 70% identical to a CDR-H3 selected from Table 8 and an amino acid sequence at least 70% identical to a CDR-H2 selected from Table 7, and any other suitable CDR-H1 polypeptide sequence, where the binding agent retains specific binding to cMET or a portion thereof. In certain embodiments, a heavy chain variable region or antigen binding portion of a binding agent described herein comprises three heavy chain CDRs consisting of an amino acid sequence at least 70% identical to a CDR-H3 selected from Table 8, an amino acid sequence at least 70% identical to a CDR-H2 selected from Table 7 and an amino acid sequence selected at least 70% identical to a CDR-H1 of Table 6. In certain embodiments, a heavy chain variable region or antigen binding portion of a binding agent described herein comprises an amino acid sequence at least 70% identical to a CDR-H3 selected from Table 8, an amino acid sequence at least 70% identical to a CDR-H2 selected from Table 7 and an amino acid sequence at least 70% identical to a CDR-H1 selected from Table 6, where the binding agent retains specific binding to cMET, or a portion thereof.

In some embodiments a binding agent comprises one or more heavy chain CDRs with at least 70%, 75%, 85%, 86%, 87%, 88%, 89%, 90%, 91%, 92%, 93%, 94%, 95%, 96%, 97%, 98%, or at least 99% identity to any one of the CDRs of Tables 6, 7 or 8. In some embodiments a binding agent or the antigen binding portion of a binding agent comprises a CDR-H1 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 6. In some embodiments a binding agent or the antigen binding portion of a binding agent comprises a CDR-H1 of any one of the sequences shown in Table 6.

TABLE 6
CDR-H1 Sequences

| ID | Clone | Amino Acid Sequence |
|---|---|---|
| SEQ ID NO: 50 | F5_P5_B9_H | GFSLTNYGVN |
| SEQ ID NO: 51 | F5_P5_B9_H | GFSLTNYG |
| SEQ ID NO: 52 | F6A_P8_E2_H | GFNINDYFMH |
| SEQ ID NO: 53 | F6A_P8_E2_H | FNINDYF |
| SEQ ID NO: 54 | F6A_P12_F12_H | GFTFTDYYMS |
| SEQ ID NO: 55 | F6A_P12_F12_H | GFTFTDYY |
| SEQ ID NO: 56 | F6B_P1_H5_H | GYTFTDYNMD |
| SEQ ID NO: 57 | F6B_P1_H5_H | YTFTDYN |
| SEQ ID NO: 58 | F6B1_P3_D12_H/ | GYTFTSYWMH |
| | F6B1P3E9_H/ | |
| | F6_B1_P1_E2_H/ | |
| | F6B_P3_E2_H/ | |
| | F6B_P2_D4_H | |
| SEQ ID NO: 59 | F6B1_P3_D12_H/ | YTFTSYW |
| | F6B1P3E9_H/ | |
| | F6_B1_P1_E2_H/ | |
| | F6B_P3_E2_H/ | |
| | F6B_P2_D4_H | |
| SEQ ID NO: 60 | Consensus | GYTFT(D/S)Y(N/W) |
| SEQ ID NO: 61 | Consensus | G(Y/F)TFT(D/S)Y |
| | | (N/W/Y)M(H/S) |

In some embodiments a binding agent or the antigen binding portion of a binding agent comprises a CDR-H2 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 7. In some embodiments a binding agent or the antigen binding portion of a binding agent comprises a CDR-H2 of any one of the sequences shown in Table 7.

TABLE 7
CDR-H2 Sequences

| ID | Clone | Amino Acid Sequence |
|---|---|---|
| SEQ ID | F5_P5_B9_H | LIWGGGDTDYNSALKS |
| NO: 62 | | |
| SEQ ID | F5_P5_B9_H | IWGGGDT |
| NO: 63 | | |
| SEQ ID | F6A_P8_E2_H | WIDPENGNTIYDPKFQG |
| NO: 64 | | |
| SEQ ID | F6A_P8_E2_H | IDPENGNT |
| NO: 65 | | |
| SEQ ID | F6A_P12_F12_H | FIRNKANGYTTKYSASVKG |
| NO: 66 | | |
| SEQ ID | F6A_P12_F12_H | IRNKANGYTT |
| NO: 67 | | |
| SEQ ID | F6B_P1_H5_H | DINPNNGGTIYNQKFKG |
| NO: 68 | | |
| SEQ ID | F6B_P1_H5_H | INPNNGGT |
| NO: 69 | | |
| SEQ ID | F6B1_P3_D12_H/ | YIKPSTDNTEYNQKFKD |
| NO: 70 | F6B1P3E9_H | |
| SEQ ID | F6B1_P3_D12_H/ | IKPSTDNT |
| NO: 71 | F6B1P3E9_H | |
| SEQ ID | F6_B1_P1_E2_H/ | YINPSTDYTEYNQKFKD |
| NO: 72 | F6B_P3_E2_H | |
| SEQ ID | F6_B1_P1_E2_H/ | INPSTDYT |
| NO: 73 | F6B_P3_E2_H | |
| SEQ ID | F6B_P2_D4_H | YINPSTDYIEYNQKFKD |
| NO: 74 | | |
| SEQ ID | F6B_P2_D4_H | INPSTDYI |
| NO: 75 | | |
| SEQ ID | Consensus | INPSTDY(I/T) |
| NO: 76 | | |
| SEQ ID | Consensus | (Y/D)I(K/N)PSTD(N/Y) |
| NO: 77 | | (T/I)EY(A/N)QKF(Q/K)(G/D) |
| SEQ ID | P3D12 VH-abb/sdr | YIKPSTDNTEYAQKFQG |
| NO: 78 | | |

In some embodiments a binding agent or the antigen binding portion of a binding agent comprises a CDR-H3 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 8. In some embodiments a binding agent or the antigen binding portion of a binding agent comprises a CDR-H3 of any one of the sequences shown in Table 8.

TABLE 8
CDR-H3 Sequences

| ID | Clone | Amino Acid Sequence |
|---|---|---|
| SEQ ID NO: 79 | F5_P5_B9_H | CARDYYGFDY |
| SEQ ID NO: 80 | F5_P5_B9_H | DYYGFDY |
| SEQ ID NO: 81 | F6A_P8_E2_H | CARGGNYLRESYYYAMDY |
| SEQ ID NO: 82 | F6A_P8_E2_H | RGGNYLRESYYYAMDY |
| SEQ ID NO: 83 | F6A_P12_F12_H | CSKDRGYFDY |
| SEQ ID NO: 84 | F6A_P12_F12_H | DRGYFDY |
| SEQ ID NO: 85 | F6B_P1_H5_H | RARGDYYGSSRYYYAMDY |
| SEQ ID NO: 86 | F6B_P1_H5_H | RGDYYGSSRYYYAMDY |
| SEQ ID NO: 87 | F6B1_P3_D12_H/ | CARSYGNYPLMDY |
| | F6B1P3E9_H | |
| SEQ ID NO: 88 | F6B1_P3_D12_H/ | SYGNYPLMDY |
| | F6B1P3E9_H & | |
| | F6_B1_P1_E2_H/ | |
| | F6B_P3_E2_H | |
| SEQ ID NO: 89 | F6_B1_P1_E2_H/ | CVRSYGNYPLMDY |
| | F6B_P3_E2_H | |
| SEQ ID NO: 90 | F6B_P2_D4_H | CARSYGNFPLMDY |
| SEQ ID NO: 91 | F6B_P2_D4_H | RSYGNFPLMDY |
| SEQ ID NO: 92 | Consensus | C(A/V)RSYGN(F/Y)PLMDY |
| SEQ ID NO: 93 | Consensus | RSYGN(F/Y)PLMDY |

In some embodiments a binding agent or the antigen binding portion of a binding agent comprises a heavy chain variable region having at least 70%, 75%, 85%, 86%, 87%, 88%, 89%, 90%, 91%, 92%, 93%, 94%, 95%, 96%, 97%, 98%, or at least 99% identity to a sequence of Table 9. In some embodiments a binding agent or the antigen binding portion of a binding agent comprises a heavy chain variable region sequence of Table 9.

TABLE 9
VARIABLE HEAVY CHAIN SEQUENCES

| ID | Clone | Amino Acid Sequence |
|---|---|---|
| SEQ ID | F5_P5_B9_H | QVQLKESGPGLVAPSQSLSITCTVSGF |
| NO: 94 | | SLTNYGVNWVRQPPGKGLEWLGLIWGG |
| | | GDTDYNSALKSRLSISKDNSKSQVFLK |
| | | METNSLQTDDTARYYCARDYYGFDYWG |
| | | QGTTLTVSS |
| SEQ ID | F6A_P12_F12_H | EVKLVESGGGLVQPGGSLRLSCATSGF |
| NO: 95 | | TFTDYYMSWVRQPPGKALEWLGFIRNK |
| | | ANGYTTKYSASVKGRFTISRDNSQSIL |
| | | YLQMNTLRAEDSATYYCSKDRGYFDYW |
| | | GQGTTLTVSS |
| SEQ ID | F6A_P8E2_H | VNSEVQLQQSGAELVRPGALVKLSCKA |
| NO: 96 | | SGFNINDYFMHWVKQRPEQGLEWIGWI |
| | | DPENGNTIYDPKFQGKASITADTSSNT |
| | | AYLQLSSLTSEDTAVYYCARGGNYLRE |
| | | SYYYAMDYWGQGTSVTVSS |
| SEQ ID | F6B_P1_H5_H | VLSEVLLQQSGPELVKPGASVKIPCKA |
| NO: 97 | | SGYTFTDYNMDWVKQSHGKSLEWIGDI |
| | | NPNNGGTIYNQKFKGKATLTVDKSSST |
| | | AYMELRSLTSEDTAVYYRARGDYYGSS |
| | | RYYYAMDYWGQGTSVTVSS |
| SEQ ID | F6B1_P3_D12_H | QVQLQQSGAELAKPGASVKMSCRASGY |
| NO: 98 | | TFTSYWMHWVKQRPGQGLDWIGYIKPS |
| | | TDNTEYNQKFKDKATLTADKSSSTAYM |
| | | QLSSLTSEDSAVYYCARSYGNYPLMDY |
| | | WGQGTSVTVSS |
| SEQ ID | F6B1P3E9_H | QVQLQQSGAELAKPGASVKMSCRASGY |
| NO: 99 | | TFTSYWMHWVKQRPGQGLDWIGYIKPS |
| | | TDNTEYNQKFKDKATLTADKSSSTAYM |
| | | QLSSLTSEDSAVYYCARSYGNYPLMDY |
| | | WGQGTSVTVSS |
| SEQ ID | F6B1_P1_E2_H | QVQLQQSGAELAKPGASVKMSCKASGY |
| NO: 100 | | TFTSYWMHWVKQRPGQGLEWIGYINPS |
| | | TDYTEYNQKFKDKATLTADKSSTTAYM |
| | | QLSSLTSEDSAVYYCVRSYGNYPLMDY |
| | | WGQGTSVTVSS |
| SEQ ID | F6B_P3_E2_H | QVQLQQSGAELAKPGASVKMSCKASGY |
| NO: 101 | | TFTSYWMHWVKQRPGQGLEWIGYINPS |
| | | TDYTEYNQKFKDKATLTADKSSTTAYM |
| | | QLSSLTSEDSAVYYCVRSYGNYPLMDY |
| | | WGQGTSVTVSS |
| SEQ ID | F6B_P2_D4_H | QVQLQQSGAELAKPGASVKMSCKASGY |
| NO: 102 | | TFTSYWMHWVKQRPGQGLEWIGYINPS |
| | | TDYIEYNQKFKDKATLTAGKSSSTAYM |
| | | QLSSLTSEDSAVYYCARSYGNFPLMDY |
| | | WGQGTSVTVSS |
| SEQ ID | Consensus | QVQLQQSGAELAKPGASVKMSC(K/R) |
| NO: 103 | | ASGYTFTSYWMHWVKQRPGQGL(E/D) |
| | | WIGYI(K/N)PSTD(Y/N)(T/I)EYN |
| | | QKFKDKATLTADKSS(S/T)TAYMQLS |
| | | SLTSEDSAVYYC(A/V)RSYGN(Y/F) |
| | | PLMDYWGQGTSVTVSS |

In some embodiments a binding agent or the antigen binding portion of a binding agent comprises a humanized heavy chain variable region having at least 70%, 75%, 85%, 86%, 87%, 88%, 89%, 90%, 91%, 92%, 93%, 94%, 95%, 96%, 97%, 98%, or at least 99% identity to a sequence of Table 10. In some embodiments a binding agent or the antigen binding portion of a binding agent comprises a humanized heavy chain variable region sequence of Table 10.

TABLE 10
Humanized P3D12 Heavy Chains

| ID | Clone | Amino Acid Sequence |
|---|---|---|
| SEQ ID | P3D12 VH | QVQLQQSGAELAKPGASVKMSCRASGYTFTSY |
| NO: 104 | | WMHWVKQRPGQGLDWIGYIKPSTDNTEYNQKF |
| | | KDKATLTADKSSSTAYMQLSSLTSEDSAVYYC |
| | | ARSYGNYPLMDYWGQGTSVTVSS |
| SEQ ID | P3D12 VH- | QVQLQQSGAEVKKPGASVKVSCKASGYTFTSY |
| NO: 105 | fra | WMHWVKQRPGQGLDWIGYIKPSTDNTEYNQKF |
| | | KDRVTLTADKSTSTAYMQLSNLISEDTAVYYC |
| | | ARSYGNYPLMDYWGQGTSVTVSS |
| SEQ ID | P3D12 VH- | QVQLVQSGAEVAKPGASVKMSCKASGYTFTSY |
| NO: 106 | ven | WMHWVKQAPGQGLDWIGYIKPSTDNTEYNQKF |
| | | KDKATLTADKSTSTAYMQLSSLRSEDTAVYYC |
| | | ARSYGNYPLMDYWGQGTTVTVSS |
| SEQ ID | P3D12 VH- | QVQLVQSGAEVKKPGASVKVSCKASGYTFTSY |
| NO: 107 | abb/sdr | WMHWVKQAPGQGLDWMGYIKPSTDNTEYAQKF |
| | | QGRVTLTADKSTSTAYMELSSLRSEDTAVYYC |
| | | ARSYGNYPLMDYWGQGTTVTVSS |
| SEQ ID | P3D12 VH- | QVQLVQSGAEVKKPGASVKVSCKASGYTFTSY |
| NO: 108 | cdr | WMHWVKQAPGQGLDWIGYIKPSTDNTEYNQKF |
| | | KDKATLTADKSTSTAYMELSSLRSEDTAVYYC |
| | | ARSYGNYPLMDYWGQGTTVTVSS |

In some embodiments a binding agent, or an antigen binding portion of a binding agent, comprises a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any one of the amino acid sequences of SEQ ID NOs: 26 to 36 (e.g., a CDR-L3 sequence selected from Table 3) and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any one of the amino acid sequences of SEQ ID NOs: 79 to 93 (e.g., a CDR-H3 sequence selected from Table 8). In some embodiments a binding agent, or an antigen binding portion of a binding agent, comprises a CDR-L3 comprising an amino acid sequence at least 70%, at least 90%, at least 95%, at least 98%, at least 99% or 100% identical to the amino acid sequence of SEQ ID NO: 34 or 35, and a CDR-H3 comprising an amino acid sequence at least 70%, at least 90%, at least 95%, at least 98%, at least 99% or 100% identical to the amino acid sequence of SEQ ID NO: 87, 88, 92 or 93.

In some embodiments a binding agent, or an antigen binding portion of a binding agent, comprises a CDR-L3 comprising an amino acid sequence at least 70%, at least 90%, or 100% identical to any one of the amino acid sequences of SEQ ID NOs: 26 to 36 (e.g., a CDR-L3 sequence selected from Table 3), a CDR-L2 comprising an amino acid sequence at least 70%, at least 90%, or 100% identical to any one of the amino acid sequences of SEQ ID NOs: 16 to 25 (e.g., a CDR-L2 sequence selected from Table 2), a CDR-H3 comprising an amino acid sequence at least 70%, at least 90%, or 100% identical to any one of the amino acid sequences of SEQ ID NOs: 79 to 93 (e.g., a CDR-H3 sequence selected from Table 8) and a CDR-H2 comprising an amino acid sequence at least 70%, at least 90%, or 100% identical to any one of the amino acid sequences of SEQ ID NOs: 62 to 78 (e.g., a CDR-H2 sequence selected from Table 7). In some embodiments a binding agent, or an antigen binding portion of a binding agent, comprises a CDR-L3 comprising an amino acid sequence at least 70%, at least 90%, or 100% identical to the amino acid sequence of SEQ ID NO: 34 or 35, a CDR-L2 comprising an amino acid sequence at least 70%, at least 90%, or 100% identical to the amino acid sequence of SEQ ID NO: 24 or 25, a CDR-H3 comprising an amino acid sequence at least 70%, at least 90%, or 100% identical to the amino acid sequence of SEQ ID NO: 87, 88, 92 or 93 and a CDR-H2 comprising an amino acid sequence at least 70%, at least 90%, or 100% identical to the amino acid sequence of SEQ ID NO: 70, 71 or 78.

In some embodiments a binding agent, or an antigen binding portion of a binding agent, comprises a CDR-L3 comprising an amino acid sequence at least 70%, at least 90%, or 100% identical to any one of the amino acid sequences of SEQ ID NOs: 26 to 36 (e.g., a CDR-L3 sequence selected from Table 3), a CDR-L2 comprising an amino acid sequence at least 70%, at least 90%, or 100% identical to any one of the amino acid sequences of SEQ ID NOs: 16 to 25 (e.g., a CDR-L2 sequence selected from Table 2), a CDR-L1 comprising an amino acid sequence at least 70%, at least 90%, or 100% identical to any one of the amino acid sequences of SEQ ID NOs: 1 to 15 (e.g., a CDR-L1 sequence selected from Table 1), a CDR-H3 comprising an amino acid sequence at least 70%, at least 90%, or 100% identical to any one of the amino acid sequences of SEQ ID NOs: 79 to 93 (e.g., a CDR-H3 sequence selected from Table 8), a CDR-H2 comprising an amino acid sequence at least 70%, at least 90%, or 100% identical to any one of the amino acid sequences of SEQ ID NOs: 62 to 78 (e.g., a CDR-H2 sequence selected from Table 7), and a CDR-H1 comprising an amino acid sequence at least 70%, at least 90%, or 100% identical to any one of the amino acid sequences of SEQ ID NOs: 50 to 61 (e.g., a CDR-H1 sequence selected from Table 6). In some embodiments a binding agent, or an antigen binding portion of a binding agent, comprises a CDR-L3 comprising an amino acid sequence at least 70%, at least 90%, or 100% identical to the amino acid sequence of SEQ ID NOs: 34 or 35, a CDR-L2 comprising an amino acid sequence at least 70%, at least 90%, or 100% identical to the amino acid sequence of SEQ ID NO: 24 or 25, a CDR-L1 comprising an amino acid sequence at least 70%, at least 90%, or 100% identical to the amino acid sequence of SEQ ID NO: 9, 10 or 15, a CDR-H3 comprising an amino acid sequence at least 70%, at least 90%, or 100% identical to the amino acid sequence of SEQ ID NO: 87 or 88, a CDR-H2 comprising an amino acid sequence at least 70%, at least 90%, or 100% identical to the amino acid sequence of SEQ ID NO: 70, 71 or 78, and a CDR-H1 comprising an amino acid sequence at least 70%, at least 90%, or 100% identical to the amino acid sequence of SEQ ID NO: 58 or 59.

In some embodiments a binding agent, or an antigen binding portion of a binding agent, comprises a heavy chain variable region comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any one of the amino acid sequences of SEQ ID NOs: 94 to 108 (e.g., a heavy chain variable region selected from Tables 9 and 10), and a light chain variable region comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any one of the amino acid sequences of SEQ ID NOs: 37 to 49 (e.g., a light chain variable region selected from Tables 4 and 5). In some embodiments a binding agent, or an antigen binding portion of a binding agent, comprises a heavy chain variable region comprising an amino acid sequence at least 90% identical to any one of the amino acid sequences of SEQ ID NOs: 104 to 108 (e.g., a heavy chain variable region selected from Table 10), and a light chain variable region comprising an amino acid sequence at least 90% identical to any one of the amino acid sequences of SEQ ID NOs: 45 to 49 (e.g., a light chain variable region selected from Table 5).

The abbreviations of “abb”, “sdr”, “fra”, “ven, “and “cdr” as used herein are explained below. The abbreviation “cdr” or “CDR” refer to a Complementarity-Determining Region. The abbreviation “abb” refers to an abbreviated CDR, for example as described in Padlan et al. (1995) FASEB J 9:133-139. In some embodiments, abbreviated CDRs are residues 27D-34, 50-55, and 89-96 in the light chain, and 31-35B, 50-58, and 95-101 in the heavy chain, which are grafted onto an appropriate human scaffold. Critical framework residues are often preserved. The abbreviation “sdr” refers to “specificity determining residues”, for example as described in Padlan et al. (1995) which are residues thought to be involved in antigen binding. The abbreviation “fra” refers to a “Frankenstein approach”, for example as described in Wu and Kabat (1992) Mol Immunol 29:1141-1146. The abbreviation “ven” refers to “Veneering”, for example as described in Padlan (1991), Mol Immunol 28:489-498.

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, Vol. 266: Computer Methods for Macromolecular Sequence Analysis (1996), ed. Doolittle, Academic Press, Inc., a division of Harcourt Brace & Co., San Diego, Calif., USA. In some embodiments an alignment program that permits gaps in the sequence is utilized to align the sequences. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

In some embodiments a binding agent, or antigen binding portion of a binding agent comprises one or more CDRs selected from a light chain variable region of Tables 4 and 5. In some embodiments a binding agent, or antigen binding portion of a binding agent comprises one or more CDRs selected from a heavy chain variable region of Tables 9 and 10. In some embodiments a binding agent, or antigen binding portion of a binding agent comprises one or more CDRs selected from a light chain variable region of Tables 4 and 5 and one or more CDRs selected from a heavy chain variable region of Tables 9 and 10. In certain embodiments, a binding agent, or antigen binding portion of a binding agent, comprises a CDR-L1, a CDR-L2, and a CDR-L3, each selected from any one of the light chain variable regions of Tables 4 and 5, and a CDR-H1, a CDR-H2, and a CDR-H3, each selected from any one of the heavy chain variable regions of Tables 9 and 10. An amino acid sequence of a CDR (e.g., a CDR-L1, CDR-L2, CDR-L3, CDR-H1, CDR-H2, and CDR-H3) can be identified within a heavy chain or light chain variable region disclosed herein by any suitable method described herein or known to those skilled in the art.

In certain embodiments, a binding agent that specifically binds to cMET comprises (i) a CDR-L1, a CDR-L2 and a CDR-L3 which are polypeptide sequences of a light chain complementarity determining region (CDR-L), where the CDR-L1 is selected from the amino acid sequences of SEQ ID NOs: 1-15, the CDR-L2 is selected from the amino acid sequences of SEQ ID NOs: 16-25, and the CDR-L3 is selected from the amino acid sequences of SEQ ID NOs: 26-36, and (ii) a CDR-H1, a CDR-H2 and a CDR-H3 which are polypeptide sequences of a heavy chain complementarity determining region (CDR-H), where the CDR-H1 is selected from the amino acid sequences of SEQ ID NOs: 50-61, the CDR-H2 is selected from the amino acid sequences of SEQ ID NOs: 62-78, and the CDR-H3 is selected from the amino acid sequences of SEQ ID NOs: 79-93.

In some embodiments a binding agent comprises one or more suitable sequences selected from Tables 1-10 wherein the selected polypeptide sequence comprises 0 to 5, 1 to 5, 0 to 10, 1 to 10, 0 to 15, or 1 to 15 amino acid modifications where an amino acid modification can be an amino acid addition, an amino acid deletion and/or an amino acid substitution. In some embodiments a binding agent comprises one or more suitable sequences selected from Tables 4, 5, 9 or 10, wherein the selected polypeptide sequence comprises 0 to 5, 1 to 5, 0 to 10, 1 to 10, 0 to 15, or 1 to 15 amino acid modifications in a framework regions or a constant region, where an amino acid modification can be an amino acid addition, an amino acid deletion and/or an amino acid substitution. In some embodiments an amino acid modification is a conservative amino acid substitution. In some embodiments, a binding agent disclosed herein comprises one or more amino acid analogues, non-native amino acids or amino acid derivatives.

In certain embodiments, a binding agent, or antigen binding portion of a binding agent comprises one or more framework regions (FR). Framework regions are often located between CDRs and/or flank CDR sequences of a heavy or light chain variable region of an antibody or binding agent. In mammals, a heavy chain variable region often comprises four framework regions and a light chain variable region often comprises four framework regions. Any suitable method can be used to identify one or more framework regions in an antibody, in a variable region of an antibody or in a binding agent. A binding agent may comprise synthetic or naturally occurring framework regions which are unmodified or modified (e.g., optimized) as discussed below.

In some embodiments a binding agent, or antigen binding portion thereof is chimeric, grafted and/or humanized. Chimeric, grafted and or humanized binding agents often comprise modified or substituted constant regions and/or framework regions while maintaining binding specificity to cMET, or a portion thereof. In some embodiments a binding agent, or antigen binding portion thereof, comprises constant regions, framework regions, or portions thereof, derived from a human antibody. In some embodiments a binding agent, or antigen binding portion thereof, comprises fully synthetic portions, one or more amino acids, or sequences of amino acids that are not found in native antibody molecules.

Naturally occurring framework regions, or portions thereof may be obtained from any suitable species. In certain embodiments the complementarity determining regions (CDRs) of the light and heavy chain variable regions of a binding agent, or an antigen binding portion thereof, is grafted into framework regions from the same, or another, species. For example, one or more framework regions of a binding agent may be derived from a rodent species (e.g., a mouse or rat) or a primate species (e.g., a human).

In certain embodiments, the CDRs of the light and/or heavy chain variable regions of a binding agent, or an antigen binding portion thereof, can be grafted to consensus human framework regions. To create consensus human framework regions, in certain embodiments, framework regions from several human heavy chain or light chain amino acid sequences can be aligned to identify a consensus sequence. In certain embodiments, the heavy chain or light chain framework regions of an antibody or binding agent are replaced with one or more framework regions, or portions thereof, from a different heavy chain or light chain variable region. In some embodiments a binding agent, or antigen binding portion thereof, comprises one or more human framework regions. In certain embodiments a binding agent, or antigen binding portion thereof, comprises at least 1, 2, 3, 4, 5, 6, 7, 8, 9 or 10 human framework regions. In some embodiments a binding agent, or antigen binding portion thereof, comprises one or more mouse framework regions. In certain embodiments a binding agent, or antigen binding portion thereof, comprises at least 1, 2, 3, 4, 5, 6, 7, 8, 9 or 10 mouse framework regions. In certain embodiments a binding agent, or antigen binding portion thereof, comprises one or more human framework regions and one or more mouse framework regions.

Methods of generating chimeric, humanized and/or optimized antibodies or binding agents, for example by modifying, substituting or deleting framework regions, or portions thereof, are known. Non-limiting examples of CDR grafting are described, e.g., in U.S. Pat. Nos. 6,180,370; 6,054,297; 5,693,762; 5,859,205; 5,693,761; 5,565,332; 5,585,089; and 5,530,101, and in Jones et al. (1986) Nature 321:522-525; Verhoeyen et al. (1988) Science 239:1534-1536, and Winter (1998) FEBS Letts. 430:92-94. Additional non-limiting examples of generating chimeric, grafted and/or humanized binding agents include U.S. Pat. Nos. 5,530,101; 5,707,622; 5,994,524; 6,245,894; Queen et al. (1988) PNAS 86:10029-10033; Riechmann et al. (1988) Nature 332:323-327; Antibody Engineering: Methods and Protocols, Vol. 248 of Methods in molecular biology, edited by Benny K. C. Lo, Springer Science & Business Media, (2004); and Antibody Engineering, Vol. 1, Roland E. Kontermann, Stefan Dübel, Edition 2, Publisher Springer Science & Business Media, (2010). In some embodiments a binding agent can be humanized by exchanging one or more framework regions, or portions thereof (e.g., one or more amino acids), with one or more framework regions, or portions thereof from a human antibody. In certain embodiments, an antibody or binding agent can be humanized or grafted by transferring one or more CDRs (e.g., 1, 2, 3, 4, 5 or all 6 CDRs) from a donor binding agent (e.g., a mouse monoclonal antibody) to an acceptor binding agent (e.g., a human antibody) while retaining the binding specificity of the donor binding agent. In certain embodiments, the process of making a chimeric, grafted or humanized binding agent comprises making one or more amino acid substitutions, additions or deletions in a constant region or framework region of a binding agent. In certain embodiments, techniques such as “reshaping”, “hyperchimerization,” or “veneering/resurfacing” can be used to produce humanized binding agents. (e.g., see Vaswami et al. (1998) Annals of Allergy, Asthma, & Immunol. 81:105; Roguska et al. (1996) Prot. Engin. 9:895-904; and U.S. Pat. No. 6,072,035). In some aspects, a binding agent is modified by a method discussed above, or by another suitable method, to reduce immunogenicity (e.g., see Gilliland et al. (1999) J. Immunol. 62 (6): 3663-71).

In certain embodiments, an amino acid sequence of a binding agent is modified to optimize binding affinity for a target (e.g., cMET), species cross-reactivity, solubility and/or function (e.g., agonist activity, or lack thereof). In some embodiments a specific combination of CDRs disclosed herein can be optimized for binding to cMET, and/or to optimize a function or characteristic of a binding agent disclosed herein. For example, a characterized light chain variable region disclosed herein (e.g., a light chain variable region of SEQ ID NO:48) can be co-expressed, using a suitable expression system, with a library of heavy chain variable regions comprising a CDR-H1 and CDR-H2 of a characterized heavy chain variable region (e.g., a heavy chain variable region of SEQ ID NO:107), where the CDR-H3 is replaced with a library of CDR-H3 sequences, which may include one or more CDR-H3 regions of Table 8, for example. The resulting light chain/heavy chain binding agents can be screened for binding to cMET and/or for a specific function. Optimized binding agents can be identified and the amino acid sequence of the CDR-H3 can be identified by a suitable method. The above screening method can be used to identify binding agents comprising specific combinations of CDRs, or specific optimized CDR sequences (e.g., CDR sequences comprising amino acid substitutions, additions or deletions) that provide a binding agent with improved binding specificity, binding affinity and/or function. Such methods of screening and optimizing binding agents are known (e.g., see Portolano et al. (1993) Journal of Immunology 150:880-887; and Clarkson et al. (1991) Nature 352:624-628). Such references teach methods of producing antibodies that bind a specific antigen by using known variable light chain, known variable heavy chains, or portion thereof (e.g., CDRs thereof) by screening a library of complementary variable regions.

In certain embodiments, a binding agent is modified to eliminate or add glycosylation sites in order to optimize affinity and/or function of a binding agent (e.g., see Co et al. (1993) Mol. Immunol. 30:1361-1367). In some embodiments the number and/or type of glycosylation sites in a binding agent is modified or altered. An N-linked glycosylation site is often characterized by the sequence Asn-X-Ser or Asn-X-Thr, where the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided in certain embodiments is a rearrangement of N-linked carbohydrate chains where one or more N-linked glycosylation sites (typically those that are naturally occurring) are eliminated and one or more new N-linked sites are created. In some embodiments a binding agent is modified by deleting one or more cysteine residues or substituting one or more cysteine residues for another amino acid (e.g., serine) as compared to an unmodified binding agent. In certain embodiments cysteine variants can be useful for optimizing expression, secretion, and/or solubility.

In certain embodiments a binding agent is modified to include certain amino acid additions, substitutions, or deletions designed or intended, for example, to reduce susceptibility of a binding agent to proteolysis, reduce susceptibility of a binding agent to oxidation, increase serum half-life and/or confer or modify other physicochemical, pharmacokinetic or functional properties of a binding agent.

In some embodiments a binding agent specifically binds to a mammalian cMET, or portion thereof. In some embodiments a binding agent specifically binds to an extracellular domain or extracellular regions of a mammalian cMET, or a portion thereof. In certain aspects, a binding agent specifically binds to a wild-type cMET produced by a cell of an unaltered (non-genetically modified) mammal found in nature. In certain aspects a binding agent specifically binds to a naturally occurring cMET variant. In certain aspects a binding agent specifically binds to a cMET comprising one or more amino acid substitutions, additions or deletions. In certain embodiments a binding agent specifically binds to a cMET produced and/or expressed on the surface of a cell of a human, non-human primate, dog, cat, or rodent (e.g., a mouse or rat). In certain embodiments, a binding agent specifically binds to one or more cMET polypeptides, or a portion thereof, having an amino acid sequence of any one of SEQ ID NOs: 109 to 113. In certain embodiments, a binding agent specifically binds to a human cMET. In certain embodiments, a binding agent specifically binds to an extracellular domain of human cMET. In certain embodiments, a binding agent specifically binds to a human cMET, and/or an extracellular domain thereof, wherein the human cMET comprises an E168 to D168 substitution (i.e., an E168D variant of cMET). In certain embodiments, a binding agent specifically binds to a human cMET, and/or an extracellular domain thereof, wherein the human cMET comprises an N375 to S375 substitution (i.e., an N375S variant of human cMET).

The term “specifically binds” refers to a binding agent that binds a target peptide in preference to binding other molecules or other peptides as determined by, for example, as determined by a suitable in vitro assay (e.g., an Elisa, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

In some embodiments a binding agent that specifically binds to cMET, or a portion thereof, is a binding agent that binds cMET, or a portion thereof (e.g., an extracellular domain of cMET), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In some embodiments a binding agent that specifically binds to cMET, or a portion thereof, is a binding agent that binds human cMET, or a portion thereof (e.g., an extracellular domain of human cMET), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In some embodiments a binding agent that specifically binds to cMET, or a portion thereof, is a binding agent that binds specifically to cMET, or a portion thereof, derived from a non-human species (e.g., a non-human primate, or rodent; e.g., a mouse or rat), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In certain embodiments, a binding agent disclosed herein specifically binds human cMET, or a portion thereof, and specifically binds to cMET, or a portion thereof, derived from a non-human primate. In certain embodiments, a binding agent disclosed herein specifically binds human cMET, or a portion thereof, and specifically binds to cMET, or a portion thereof, derived from a rodent (e.g., a mouse or rat). In certain embodiments, a binding agent (i) specifically binds to a human cMET, or portion thereof (e.g., an extracellular domain of human cMET) with a KD of 10 nM or less, or 1 nM or less, and (ii) specifically binds to a rat or mouse cMET, or portion thereof (e.g., an extracellular domain of rat or mouse cMET) with a KD of 100 nM or less, 90 nM or less, 80 nM or less, 70 nM or less, 60 nM or less, 50 nM or less, 40 nM or less, 30 nM or less, 20 nM or less or 10 nM or less.

In certain embodiments, a binding agent comprises one or more functional characteristics. Accordingly, a binding agent can be described structurally and functionally (e.g., by what it does, or by what it is capable of doing). Binding agents disclosed herein can bind specifically to an extracellular portion of cMET, for example, an extracellular portion of cMET present on the surface of a cell. In some embodiments a cell is a human cancer cell or human neoplastic cell that expresses cMET. In certain embodiments, binding agents disclosed herein, upon binding cMET on the surface of a cell, induce internalization of cMET. The ability of a cMET binding agent to induce internalization and/or degradation of cMET provides an advantage over other cMET binding agents that lack this ability. A cMET binding agent-drug conjugate that induces internalization and/or degradation of cMET after binding provides for localized intracellular delivery of a cytotoxic drug. Further, in some embodiments, a binding agent-drug conjugate is configured to release a PBD toxin from the binding agent only after internalization, for example by means of a lysosomal enzyme cleavage site integrated into a linking group. Accordingly, binding agent-drug conjugates described herein can deliver a toxin specifically to the inside of a cancer cell that expresses cMET while minimizing non-specific cytotoxicity to healthy cells in a subject. Consequently, the anti-cMET binding agent-drug conjugates described herein provide for higher efficacy (e.g., target-specific cytotoxicity) and less adverse side effects (e.g., less non-specific cytotoxicity). Accordingly, in certain embodiments, a binding agent-drug conjugate comprises a binding agent that specifically binds cMET on a cell surface of a cell and induces internalization of cMET after binding. In some embodiments, a binding agent binds specifically to cMET, or a portion thereof, and induces degradation of cMET. Accordingly, in certain embodiments, a binding agent-drug conjugate comprises a binding agent that specifically binds cMET on a cell surface of a cell and induces internalization and/or degradation of cMET after binding. Internalization and/or degradation of a cell surface-bound receptor induced by binding of a ligand or binding agent is a known biological process that can be detected, measured, and/or quantitated using a suitable assay known in the art. Accordingly, the ability of a binding agent to induce cMET internalization and/or degradation can be determined, without undue experimentation, by use of a suitable experimental assay. Accordingly, in some embodiments, a binding agent described herein is a binding agent that binds specifically to cMET, or a portion thereof, on a cell surface and induces internalization and/or degradation of cMET.

Activation of cMET by binding of its cognate ligands is implicated in tumor growth, angiogenesis, and metastasis. Agonist anti-cMET antibodies often mimic ligand binding by cross-linking cMET receptors and inducing cMET activation. Accordingly, a binding agent that binds cell-surface cMET without activating the cMET receptor is better suited for anti-cancer therapy applications. In some embodiments, a binding agent of a binding agent-drug conjugate binds specifically to cMET, or a portion thereof, on a cell surface and does not detectably induce or promote signaling (e.g., tyrosine kinase activity). In some embodiments, a binding agent of a binding agent-drug conjugate binds specifically to cMET, or a portion thereof, on a cell surface and does not substantially activate cMET (e.g., tyrosine kinase activity). In certain embodiments, an anti-cMET binding agent disclosed herein does not have detectable cMET agonist activity. In certain embodiments, an anti-cMET binding agent lacks agonistic activity upon binding cMET on a cell surface and/or fails to induce or promote detectable tyrosine kinase activity upon binding to cMET on a cell surface. In some embodiments, an anti-cMET binding agent is a cMET antagonist. In certain embodiments, an anti-cMET binding agent decreases, inhibits, reduces, blocks or prevents signaling through a cMET receptor and/or decreases, inhibits, reduces, blocks or prevents a cMET receptor from inducing or promoting detectable tyrosine kinase activity. In some embodiments, an anti-cMET binding agent disclosed herein decreases, inhibits, reduces, prevents or blocks cMET from binding to its native cognate ligand (e.g., hepatocyte growth factor, or an isoform thereof).

In some embodiments a binding agent comprises a label. As used herein, the terms “label” or “labeled” refers to incorporation of a detectable marker, e.g., by incorporation of a labeled amino acid or attachment to a polypeptide of biotin moieties that can be detected by labeled avidin (e.g., streptavidin containing a fluorescent marker or enzymatic activity that can be detected by optical or colorimetric methods). In certain embodiments, a label or marker can be attached to a binding agent to generate a therapeutic or diagnostic agent. A binding agent can be attached covalently or non-covalently to any suitable label or marker. Various methods of labeling polypeptides and glycoproteins are known to those skilled in the art and can be used. Non-limiting examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹²⁵I, ¹³¹I), fluorescent labels, enzymatic labels (e.g., horseradish peroxidase, β-galactosidase, luciferase, alkaline phosphatase), chemiluminescent labels, a metallic label, a chromophore, an electro-chemiluminescent label, a phosphorescent label, a quencher (e.g., a fluorophore quencher), a fluorescence resonance energy transfer (FRET) pair (e.g., donor and acceptor), a dye, an enzyme substrate, a small molecule, a mass tag, quantum dots, nanoparticles, biotinyl groups, predetermined polypeptide epitopes recognized by a secondary reporter (e.g., leucine zipper pair sequences, binding sites for secondary antibodies, metal binding domains, epitope tags), the like or combinations thereof.

In some embodiments a binding agent comprises a suitable carrier. A binding agent can be attached covalently or non-covalently to a suitable carrier. In certain embodiments, a carrier is an agent or molecule that alters or extends the in vivo half-life of a binding agent, or improves it pharmacokinetic characteristics. Non-limiting examples of a carrier include polyethylene glycol, glycogen (e.g., by glycosylation of a binding agent), dextran, and a carrier or vehicle described in U.S. Pat. No. 6,660,843, the like or combinations thereof.

### Payloads

### PBD Toxins

In certain embodiments, a binding agent-drug conjugate comprises a binding agent described herein (e.g., a binding agent that specifically binds to cMET) and a payload (e.g., a cytotoxic payload). A payload of a binding agent-drug conjugate is often covalently linked to a binding agent. In some embodiments, a payload comprises a pyrrolobenzodiazepine (PBD) toxin. In some embodiments, a payload comprises a linking group or a suitable linker. In some embodiments, a payload comprises a pyrrolobenzodiazepine (PBD) toxin and a linking group. In certain embodiments a payload comprises a pyrrolobenzodiazepine (PBD) toxin and a linking group, where the pyrrolobenzodiazepine toxin is covalently linked to a linking group, and the linking group is covalently linked to a binding agent described herein.

Non-limiting examples of PBD toxins and methods of making PBD toxins are described in the following patent application publications: US 2011/0256157, WO/2015/052322, US 2016/0106861, US 2007/0072846, US 2011/0201803, US 2010/0113425, US 2008/0167293, US 2014/0127239, US 2015/0158869, US 2015/0344482, US 2015/0111880, US 2015/0315196, US 2016/0015828, US 2014/0088089, US 2013/0035484, US 2011/0196148, US 2013/0028919, US 2013/0059800, US 2014/0274907, US 2014/0275522, US 2014/0234346, US 2013/0266595, US 2014/0302066, US 2014/0286970, US 2014/0294868, US 2016/0144052, US 2016/0031887, US 2014/0120118, US 2016/0250344, WO/2017/137553, WO/2017/137555 and WO/2017/186894, the entire contents of which are incorporated herein by reference in their entirety.

In some embodiments, a pyrrolobenzodiazepine toxin comprises the structure of chemical formula I:

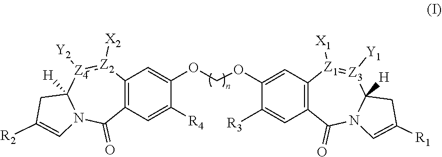

where Z₁ and Z₂ are both N; Z₃ and Z₄ are both C; the double-dash lines [symbol] represent a single bond or a double bond; n is 1 to 12; each of R₃ and R₄ are independently H, or a C₁₋₄ alkoxyl; and each of R₁ and R₂ are independently selected from the group consisting of H, C₁₋₅ alkyl, C₃₋₆ cycloalkyl, C₂₋₅ alkenyl, and a phenyl optionally substituted with R₅, where R₅ is selected from the group consisting of —NH₂, —NHR₆, and a piperazinyl substituted with R₇ having the structure

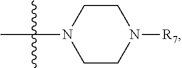

where R₆ comprises a linking group, and R₇ is null, or a C₁₋₅ alkyl; X₁ is null, a protecting group, or comprises a linking group; X₂ is null, a protecting group, or comprises a linking group; only one of X₁, X₂, R₁, and R₂ comprises a linking group; and each of Y₁ and Y₂ are independently either null, OH, or SO₃; provided that: (i) when X₁ comprises a linking group, Z₁ [symbol] Z₃ is N—C, (ii) when X₂ comprises a linking group, Z₂ [symbol] Z₄ is N—C, (iii) when X₁ comprises the protecting group, Z₁ [symbol] Z₃ is N—C, and (iv) when X₂ comprises the protecting group, Z₂ [symbol] Z₄ is N—C.

In certain embodiments, a PBD toxin comprises only one linking group. For example, in chemical formula I, only one of X₁, X₂, R₁, and R₂ may comprise a linking group. For example, where X₁ comprises a linking group, X₂, R₁, and R₂ do not comprise a linking group.

In certain embodiments of the PBD toxin of chemical formula I, n is 1-12. In certain embodiments of the PBD toxin of chemical formula I, n is 1-10, 1-9, 1-7, 1-5, or 1-3. In certain embodiments of the PBD toxin of chemical formula I, n is 1, 2, 3, 4, 5, 6, 7, 8, 9, 10, 11 or 12. In some embodiments, n is 1, 3 or 5. In some embodiments, n is 3 or 5.

In certain embodiments of the PBD toxin of chemical formula I, R₃ and R₄ are independently C₁₋₄ alkoxyl. In certain embodiments of the PBD toxin of chemical formula I, R₃ and R₄ are independently selected from —O—CH₂CH₃ or —O—CH₃. In certain embodiments of the PBD toxin of chemical formula I, R₃ and R₄ are both —O—CH₃.

In certain embodiments of the PBD toxin of chemical formula I, R₁ and R₂ are independently selected from the group consisting of H, C₁₋₅ alkyl, C₃-C₆ cycloalkyl, and C₂₋₅ alkenyl. R₁ and R₂ can be the same or different. In some embodiments, R₁ and R₂ are independently selected from a C₁-C₃ alkyl and a C₂-C₃ alkenyl. In certain embodiments, R₁ and R₂ are independently selected from —CH₂CH₂CH₃ and —CH₃. In certain embodiments, both R₁ and R₂ are —CH₂CH₂CH₃ or —CH₃.

In certain embodiments of the PBD toxin of chemical formula I, R₁ and R₂ are independently selected from a C₃-C₆ cycloalkyl, and a phenyl optionally substituted with R₅, where R₅ is selected from the group consisting of —NH₂, —NHR₆, and a piperazinyl substituted with R₇ having the structure

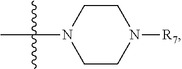

where R₆ comprises a linking group, and R₇ is null, or a C₁₋₅ alkyl. In certain embodiments, R₁ and R₂ are different and independently selected from a (i) a C₃-C₆ cycloalkyl, and (ii) a phenyl optionally substituted with R₅, where R₅ is selected from —NH₂, and —NHR₆, where R₆ comprises a linking group. In certain embodiments, R₁ and R₂ are different and independently selected from a (i) a C₃ cycloalkyl, and (ii) a phenyl substituted with —NH₂, or —NHR₆, where R₆ comprises a linking group. In certain embodiments, R₁ and R₂ are different and independently selected from a (i) a phenyl optionally substituted with R₅, where R₅ is selected from —NH₂, and —NHR₆, where R₆ comprises a linking group and (ii) a piperazinyl substituted with R₇ having the structure

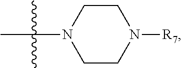

where R₇ is null, or a C₁-C₂ alkyl. In certain embodiments, R₁ and R₂ are different and independently selected from a (i) a phenyl substituted with R₅, where R₅ is —NH₂, and —NHR₆, where R₆ comprises a linking group and (ii) a piperazinyl substituted with R₇ having the structure

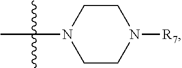

where R₇ is —CH₃. In certain embodiments, R₂ is phenyl substituted with 4-methylpiperazin-1-yl.

In certain embodiments of the PBD toxin of chemical formula I, X₁ is null, Y₁ is null, Z₁ [symbol] Z₃ is N═C, X₂ is null, Y₂ is null and Z₂ [symbol] Z₄ is N═C. In certain embodiments of the PBD toxin of chemical formula I, X₁ comprises the linking group, Y₁ is a OH, Z₂ [symbol] Z₄ is N═C, X₂ is null, and Y₂ is null. In certain embodiments of the PBD toxin of chemical formula I, X₁ comprises the linking group, Y₁ is a OH, Z₂ [symbol] Z₄ is N—C, X₂ is a protecting group, and Y₂ is OH.

In some embodiments, a PBD toxin comprises the structure of chemical formula VII shown below:

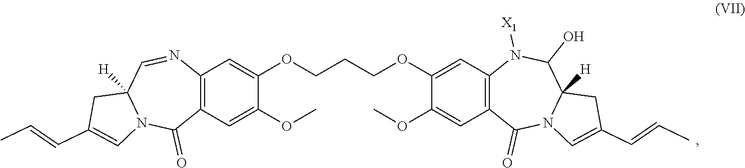

where X₁ comprises the linking group.

In some embodiments, a PBD toxin comprises the structure of chemical formula VIII shown below:

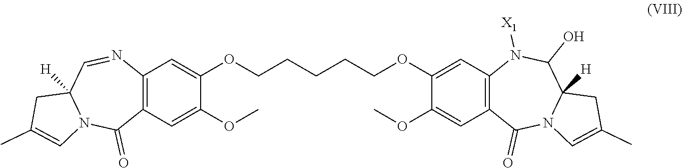

where X₁ comprises the linking group.

In some embodiments, a PBD toxin comprises the structure of chemical formula IX shown below:

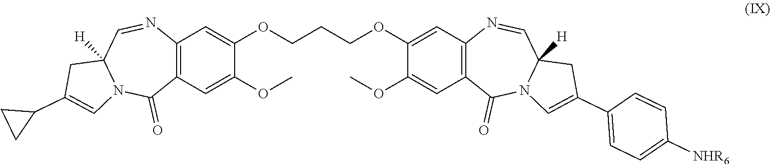

where R₆ comprises the linking group.

In some embodiments, a PBD toxin comprises the structure of chemical formula X shown below:

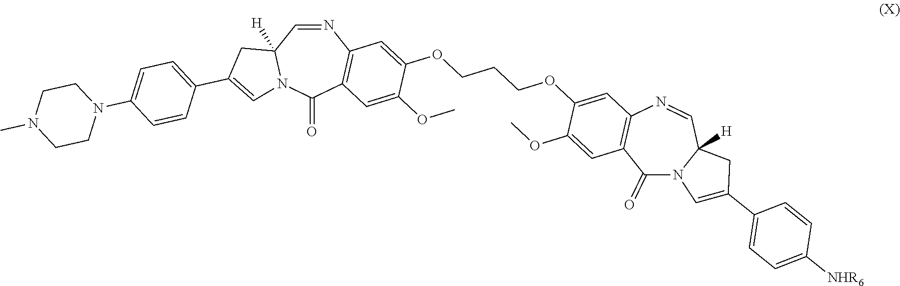

where R₆ comprises the linking group.

In some embodiments, a PBD toxin is attached (e.g., covalently linked) to a linking group by a suitable bond, moiety or group. In some embodiments, a PBD toxin is attached (e.g., covalently linked) to a linking group by a carbonyl linkage or an amide linkage. In some embodiments, a PBD toxin is attached (e.g., covalently linked) to a linking group by a carbamate group. In some embodiments, a PBD toxin is attached (e.g., covalently linked) to a linking group by an amide group. Non-limiting examples of attaching PBD toxin to a linking group are described in US 2017/0002096, US 2016/0331842, US 2015/0250896, US 2017/0080103, US 2016/0136300, US 2017/0152274, US 2015/0209444, US 2013/0274091, US 2017/0095570, US 2017/0157264, US 2015/0125474, US 2011/0256157, WO/2015/052322, US 2016/0106861, US 2007/0072846, US 2011/0201803, US 2010/0113425, US 2008/0167293, US 2014/0127239, US 2015/0158869, US 2015/0344482, US 2015/0111880, US 2015/0315196, US 2016/0015828, US 2014/0088089, US 2013/0035484, US 2011/0196148, US 2013/0028919, US 2013/0059800, US 2014/0274907, US 2014/0275522, US 2014/0234346, US 2013/0266595, US 2014/0302066, US 2014/0286970, US 2014/0294868, US 2016/0144052, US 2016/0031887, US 2014/0120118, US 2016/0250344, WO/2017/137553, WO/2017/137555 and WO/2017/186894, the entire contents of which are incorporated herein by reference in their entirety.

The term “null” as used herein means that an indicated moiety is absent from a structure, however, the indicated moiety may be replaced or occupied by one or more hydrogen atoms to complete a required valency. Further, in reference to any structure shown herein, one or more hydrogens may be present to complete a required valency of a carbon, nitrogen or oxygen atom shown in a structure. Accordingly, where not explicitly indicated, one or more hydrogen atoms may be present.

### Linking Groups

In some embodiments, a payload comprises a linking group that, in part, facilitates a linkage between a binding agent and a PBD toxin. In certain embodiments, any suitable linking group can be used to link a PBD toxin to a binding agent. Non-limiting examples of linking groups and methods a making linking groups are described in WO/2015/052322, US 2015/0158869, US 2015/0344482, US 2014/0127239, US 2017/0002096, US 2016/0331842, US 2015/0250896, US 2017/0080103, US 2016/0136300, US 2017/0152274, US 2015/0209444, US 2013/0274091, US 2017/0095570, US 2017/0157264 and US 2015/0125474, which are incorporated herein by reference in their entirety. In some embodiments, a linking group comprises a C1-C20 alkyl, a C1-C20 alkenyl, a C1-C20 alkoxyl, one or more amino acids or amino acid derivatives, a peptide comprising 1 to 20 amino acids, a phenyl group, a suitable polymer (e.g., polyethylene glycol), or a combination thereof.

In some embodiments, a linking group comprises the structure of chemical formula A:

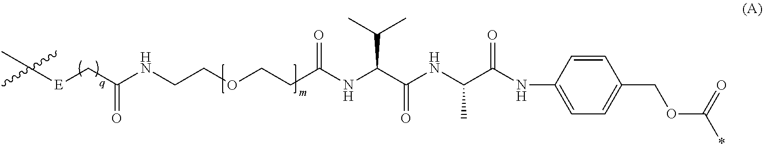

wherein the asterisk indicates the point of attachment of the linking group to a pyrrolobenzodiazepine toxin, the wavy line indicates the point of attachment of the linking group to a binding agent, m is 0 to 20, q is 0 to 10 and E is a connecting group. In some embodiments of the linking group of chemical formula A, m is 1 to 20, 1 to 10, 1 to 8, 1 to 6, 1 to 4, 2 to 8 or 4 or 8. In some embodiments of the linking group of chemical formula A, m is selected from 1, 2, 3, 4, 5, 6, 7, 8, 9 or 10. In some embodiments of the linking group of chemical formula A, q is 1 to 10, 1 to 8, 1 to 6, or 1 to 4. In some embodiments of the linking group of chemical formula A, q is selected from 0, 1, 2, 3, 4, 5, 6, 7, 8, 9 or 10. In some embodiments of the linking group of chemical formula A, q is 0, 1 or 2. 18. In some embodiments of the linking group of chemical formula A, m is 8 and q is 2.

In some embodiments, a linking group comprises the structure of chemical formula B:

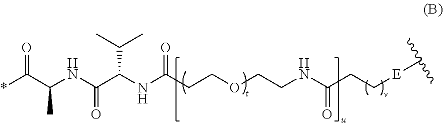

wherein the asterisk indicates the point of attachment of the linking group to a pyrrolobenzodiazepine toxin, the wavy line indicates the point of attachment of the linking group to a binding agent, v is 0 to 10, and u is 0 or 1, wherein when u is 1, tis 1 to 10, and E is a connecting group. In some embodiments of the linking group of chemical formula B, v is 1 to 10, 1 to 8, 1 to 4, or 0 to 4. 21. In some embodiments of the linking group of chemical formula B, v is selected from 0, 1, 2, 3, 4, 5, 6, 7 or 8. In some embodiments of the linking group of chemical formula B, when u is 1, tis 1 to 8, 1 to 5, 1 to 4, or 2 to 5. In some embodiments of the linking group of chemical formula B, when u is 1, t is selected from 0, 1, 2, 3, 4, 5, 6, 7, 8, 9 or 10. In some embodiments of the linking group of chemical formula B, tis 8, u is 1, and vis 2. In some embodiments of the linking group of chemical formula B, u is 0, and v is 4.

The connecting group E of chemical formulas A and B can comprise any suitable bond, linker or moiety non-limiting examples of which include a disulfide bond, a thioether bond, a thioester bond, an amide bond, an amine, a ketone, a carboxylate ether, a carbamate, an ester, a thioester, the like, or a combination thereof. In certain embodiments, E comprises a covalent linkage between the linking group and the binding agent. In some embodiments, E comprises a covalent bond. In some embodiments, E comprises a reacted moiety that remains after a suitable conjugation reaction is conducted. A multitude of conjugation reactions are known in the art, any one of which can be used to covalently link a linking group disclosed herein to a binding agent disclosed herein. Any suitable conjugation chemistry can be used to covalently attach a linking group to a binding agent, either stochastically or site-specifically, non-limiting examples of which include a conjugation reaction described in Shan S. Wong (Published Jun. 18, 1991) Chemistry of Protein Conjugation and Cross-Linking, CRC Press; Greg T. Hermanson (Copyright 2013) Bioconjugate Techniques, Third Edition, Elsevier Inc.; and Thiol-X Chemistries in Polymer and Materials Science, RSC Polymer Chemistry Series No. 6 (2013) Edited by Andrew B. Lowe and Christopher N. Bowman, RCS Publishing, WO/2015/052322, US 2015/0158869, US 2015/0344482, US 2014/0127239, US 2017/0002096, US 2016/0331842, US 2015/0250896, US 2017/0080103, US 2016/0136300, US 2017/0152274, US 2015/0209444, US 2013/0274091, US 2017/0095570, US 2017/0157264 and US 2015/0125474, the entire contents of which are incorporated herein by reference in their entirety. Other non-limiting examples of conjugating a payload or linking group to a binding agent include reacting an amine or amino group with an N-hydroxysuccinimide (NHS) ester, succinimidyl succinate, succinimidyl succinamide, succinimidyl propionate, succinimidyl carbonate, oxycarbonylimidazole, nitrophenyl carbonates, trichlorophenyl carbonate, tresylate, maleic anhydride, methylmaleic anhydride, an imidoester, a pentafluorophenyl (PFP) ester, a hydroxymethyl phosphine, an oxirane or any other carbonyl moiety; reacting a carboxyl moiety with a carbodiimide; reacting a sulfhydryl moiety with a maleimide, a haloacetyl, a pyridyldisulfide, orthopyridyldisulfide and/or a vinyl sulfone; reacting an aldehyde moiety with a hydrazine or hydrazide; reacting any non-selective group with diazirine and/or aryl azide; reacting a hydroxyl moiety with isocyanate; reacting a hydroxylamine moiety with a carbonyl moiety; the like and combinations thereof.

Accordingly, E is often defined by a chemistry used to conjugate a linking group to a binding agent. In some embodiments, E comprises a suitable moiety configured to attach a linking group to a binding agent. In some embodiments, a linking group is covalently linked to a binding agent by means of a suitable sulfhydryl-sulfhydryl reaction, for example by use of a maleimide or pyridyldithiol reactive group that reacts with a reduced cysteine to form stable thioether bond. Additional non-limiting examples of reactive sulfhydryl reactive moieties include a haloacetyls, aziridines, acryloyls, arylating agents, vinylsulfones, a pyridyl disulfide, and TNB-thiol. In certain embodiments, a binding agent is connected to E by a thioether bond formed between a cysteine thiol residue (e.g., a thiol) of the binding agent and E. Accordingly, in certain embodiments, E comprises a disulfide bond or thioether bond. In some embodiments, for example where a maleimide reaction is used to covalently link a binding agent to a linking group, E comprises the structure of chemical formula C:

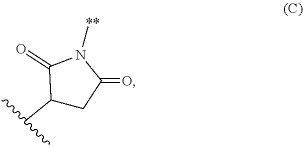

wherein the wavy line indicates the point of attachment to the binding agent and the double asterisk (**) indicates the point of attachment to the linking group. In certain embodiments the double asterisk of chemical formula C represents a thioether bond.

A payload, linking group or connecting group can be conjugated stochastically or site-specifically to any suitable amino acid of a binding agent. In some embodiments, a payload, linking group or connecting group is conjugated to one or more suitable cysteines of a binding agent. In some embodiments, a payload, linking group or connecting group is conjugated to one or more suitable lysine residues of a binding agent. In certain embodiments, one or more amino acids of a binding agent are substituted with an amino acid that is suitable for conjugation to a payload, linking group or connecting group. Non-limiting examples of amino acids that can be substituted with a thiol containing amino acid residue or a lysine residue include A118, S119, S239, V282, T289, N361, and V422 of an IgG2, S115, S252, V289, T306, and N384 of an IgG1, or a corresponding position in an IgG1, IgG2, IgG3 or IgG4. Incorporation of cysteines into antibodies by mutagenesis allows for direct conjugation of a payload, linking group or connecting group to specific sites on the antibody, for example via a disulfide bond or thioether bond. For example, one or more amino acids of a binding agent can be substituted with a cysteine, where the cysteine can be used for site-specific conjugation of a payload, linking group or connecting group using a suitable chemical reaction. Any suitable amino acid of a constant region of an antibody can be mutated to a cysteine or lysine for site-specific conjugation to a payload, linking group or connecting group. The stability of an antibody drug conjugate resulting from a site-specific conjugation can be assessed by methods known in the art.

In some embodiments, a linking group comprises a suitable enzyme cleavage site. In certain embodiments, an enzyme cleavage site comprises an enzyme recognition site of a mammalian protease. Accordingly, in some embodiments, a linking group, or portion thereof, is cleavable by a mammalian protease. A linking group may be cleaved by an enzyme present at or near a target site (e.g., at or near a cMET protein). An enzyme present at or near a target site may be intracellular, membrane bound, membrane associated or extracellular (e.g., secreted). For example a linking group may be configured to be cleaved by a cell surface protease, a secreted protease, or an intracellular protease (e.g., a lysosomal protease). Non-limiting examples of enzyme cleavage sites include a protease recognition site of a lysosomal cysteine protease and/or a lysosomal aspartic protease. Non-limiting examples of lysosomal proteases include cathepsin B, C, H, I, J, K, L, M, N, O, P, S, T and X, and cathepsin D, E, F, G, and/or cathepsin A (carboxypeptidase A).

### Protecting Groups

In some embodiments, a PBD toxin comprises a suitable protecting group. Non-limiting examples of protecting groups and method of making protecting groups are described in the following patent application publications: US 2011/0256157, WO/2015/052322, US2011/0201803, US2008/0167293, US2014/0127239, US2015/0158869, US2015/0344482, US2015/0315196, US2015/0315196, US2014/0302066, US2006/0264622 and US2015/0133435, the entire contents of which are incorporated herein by reference in their entirety.

In some embodiments, a protecting group comprises the structure of chemical formula D below:

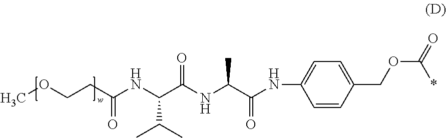

wherein the asterisk indicates a point of attachment to a pyrrolobenzodiazepine toxin; and w is 0 to 10. In some embodiments, w is 0 to 8, 0 to 6, 0 to 4, 1 to 10, 1 to 8, 1 to 5, or 1 to 4. In certain embodiments, w is selected from 0, 1, 2, 3, 4, 5, 6, 7, 8, 9 and 10. In some embodiments, w is 2.

In some embodiments, a protecting group is removable. In certain embodiments, a protecting group is cleavable using a suitable chemistry.

In some embodiments, a payload comprises a structure of chemical formula II:

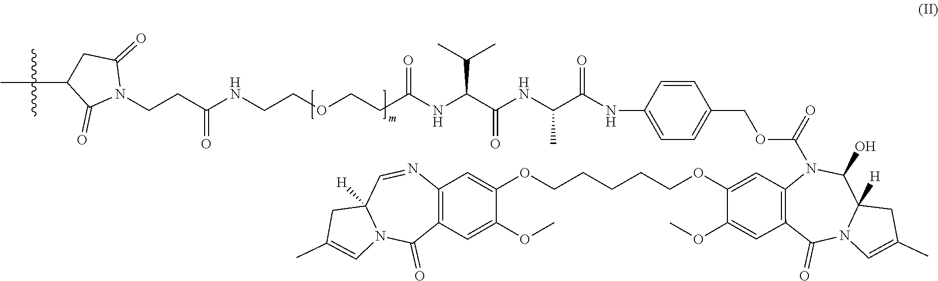

wherein m is 8 and the wavy line indicates the point of attachment to the binding agent.

In some embodiments, a payload comprises a structure of chemical formula III:

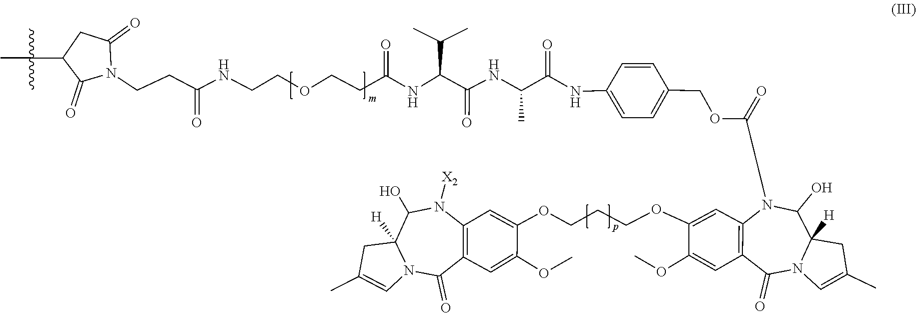

wherein m is 8, p is 1 or 3, X₂ is null, or is a protecting group and the wavy line indicates the point of attachment to the binding agent. In certain embodiments, a payload comprises a structure of chemical formula IV:

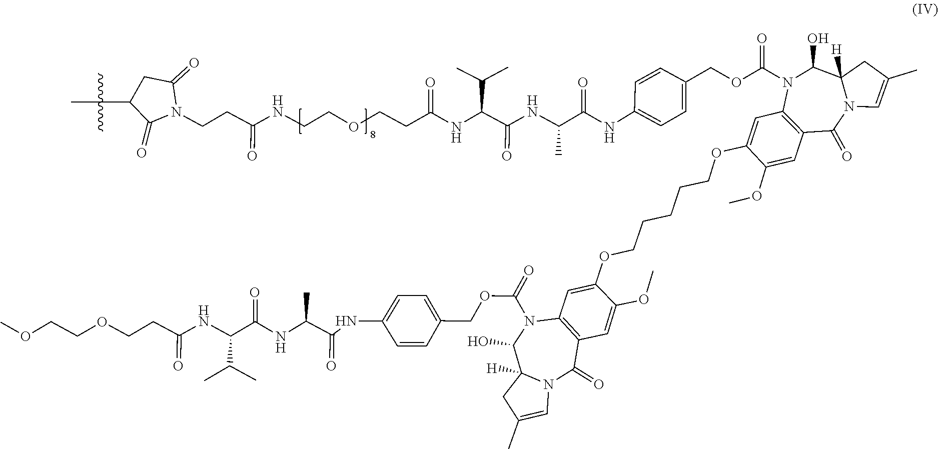

where the wavy line indicates the point of attachment to the binding agent.

In some embodiments, a payload comprises a structure of chemical formula V:

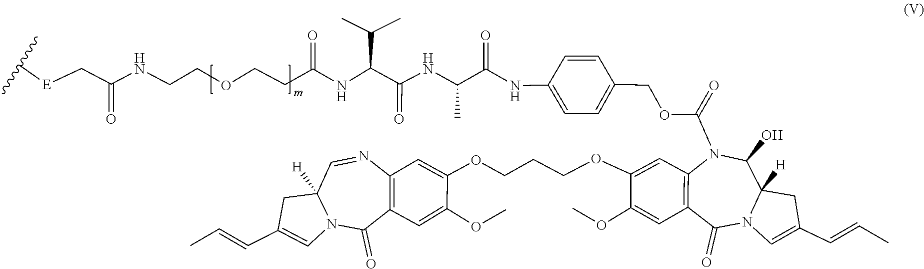

wherein m is 8, E is a suitable connecting group and the wavy line indicates the point of attachment to the binding agent. In some embodiments, E comprises a succinamide moiety of the structure C:

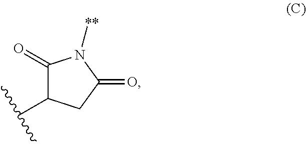

wherein the wavy line indicates the point of attachment to the binding agent and the double asterisk indicates the point of attachment to the payload of chemical formula V. The payload of chemical formula V comprising the connecting group of structure C is sometimes referred to herein as chemical formula XI.

In some embodiments, a payload comprises a structure of chemical formula VI:

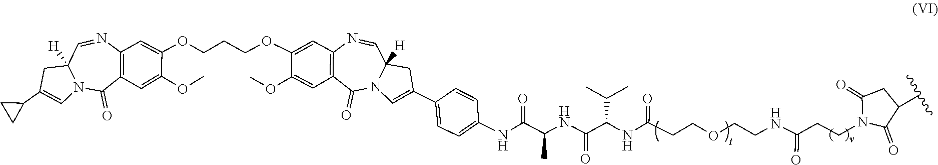

wherein tis 8, v is 1 and the wavy line indicates the point of attachment to the binding agent.

In some embodiments, a payload comprises a structure of chemical formula VII:

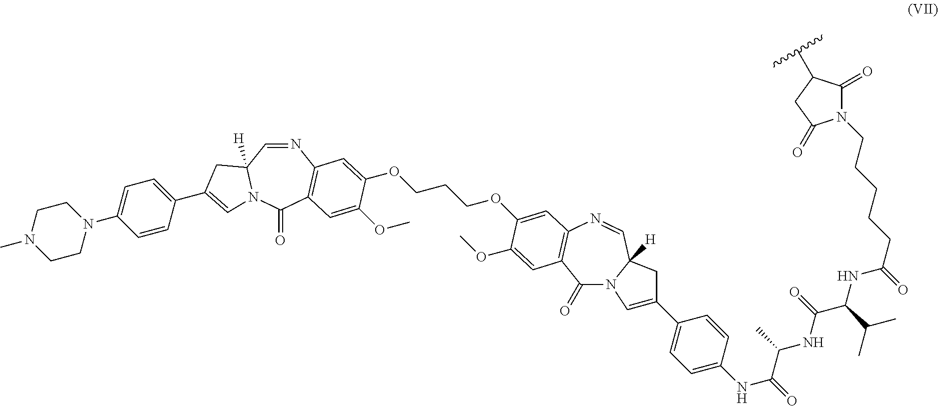 [SMILES: *C1CC(=O)N(CCCCCC(=O)N[C@H](C(=O)N[C@@H](C)C(=O)Nc2ccc(C3=CN4C(=O)c5cc(OC)c(OCCCOc6cc7c(cc6OC)C(=O)N6C=C(c8ccc(N9CCN(C)CC9)cc8)C[C@H]6C=N7)cc5N=C[C@@H]4C3)cc2)C(C)C)C1=O]

wherein the wavy line indicates the point of attachment to the binding agent.

In some embodiments, a binding agent-drug conjugate comprises a payload comprising a structure selected from any one of chemical formulas II, III, IV, V, VI, VII and XI, and a binding agent comprising a CDR-L1 selected from the amino acid sequences of SEQ ID NOs: 2, 4, 6, 8, 10, 12 and 14, a CDR-L2 selected from the amino acid sequences of SEQ ID NOs: 17, 19, 21, 23 and 25, a CDR-L3 selected from the amino acid sequences of SEQ ID NOs: 27, 29, 31, 33 and 35, a CDR-H1 selected from the amino acid sequences of SEQ ID NOs: 51, 53, 55, 57 and 59, a CDR-H2 selected from the amino acid sequences of SEQ ID NOs: 63, 65, 67, 69, 71, 73 and 75, and a CDR-H3 selected from the amino acid sequences of SEQ ID NOs: 80, 82, 84, 86, 88, 91 and 93.

In some embodiments, a binding agent-drug conjugate comprises a payload comprising a structure selected from any one of chemical formulas II, III, IV, V, VI, VII and XI, and a binding agent comprising a CDR-L1 comprising the amino acid sequence of SEQ ID NO: 10 or 14, a CDR-L2 comprising the amino acid sequence of SEQ ID NO: 21, a CDR-L3 comprising the amino acid sequence of SEQ ID NO: 35, a CDR-H1 comprising the amino acid sequences of SEQ ID NO: 59, a CDR-H2 comprising the amino acid sequence of SEQ ID NO: 71, and a CDR-H3 comprising the amino acid sequence of SEQ ID NO: 88.

In some embodiments, a binding agent-drug conjugate comprises a payload comprising a structure selected from any one of chemical formulas II, III, IV, V, VI, VII and XI, and a binding agent comprising a CDR-L1 comprising the amino acid sequence of SEQ ID NO: 9, a CDR-L2 comprising the amino acid sequence of SEQ ID NO: 24, a CDR-L3 comprising the amino acid sequence of SEQ ID NO: 34, a CDR-H1 comprising the amino acid sequences of SEQ ID NO: 58, a CDR-H2 comprising the amino acid sequence of SEQ ID NO: 70, and a CDR-H3 comprising the amino acid sequence of SEQ ID NO: 87.

In some embodiments, a binding agent-drug conjugate comprising a payload comprising a structure selected from any one of chemical formulas II, III, IV, V, VI, VII and XI, and a binding agent comprising a variable light chain region having at least 90% sequence identity to an amino acid sequence selected from the amino acid sequences of SEQ ID NOs: 37-44, and a variable heavy chain region having at least 90% sequence identity to an amino acid sequence selected from the amino acid sequences of SEQ ID NOs: 94-103.

In some embodiments, a binding agent-drug conjugate comprising a payload comprising a structure selected from any one of chemical formulas II, III, IV, V, VI, VII and XI, and a binding agent comprising a variable light chain region having an amino acid sequence selected from the amino acid sequences of SEQ ID NOs: 37-44, and a variable heavy chain region having an amino acid sequence selected from the amino acid sequences of SEQ ID NOs: 94-103.

In some embodiments, a binding agent-drug conjugate comprising a payload comprising a structure selected from any one of chemical formulas II, III, IV, V, VI, VII and XI, and a binding agent comprising a variable light chain region having at least 90% sequence identity to an amino acid sequence selected from the amino acid sequences of SEQ ID NOs: 45-49, and a variable heavy chain region having at least 90% sequence identity to an amino acid sequence selected from the amino acid sequences of SEQ ID NOs: 104-108.

In some embodiments, a binding agent-drug conjugate comprising a payload comprising a structure selected from any one of chemical formulas II, III, IV, V, VI, VII and XI, and a binding agent comprising a variable light chain region comprising an amino acid sequence selected from the amino acid sequences of SEQ ID NOs: 45-49, and a variable heavy chain region comprising an amino acid sequence selected from the amino acid sequences of SEQ ID NOs: 104-108.

### Pharmaceutical Compositions

In some embodiments, a composition or pharmaceutical composition comprises a binding agent-drug conjugate described herein. In some embodiments, a pharmaceutical composition comprises a binding agent-drug conjugate and a pharmaceutically acceptable excipient, diluent, additive or carrier.

A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants. In particular, pharmaceutical compositions can comprise any suitable carrier, formulation, or ingredient, the like or combinations thereof as listed in “Remington: The Science And Practice Of Pharmacy” Mack Publishing Co., Easton, PA, 19^{th} Edition, (1995) (hereafter, Remington '95), or “Remington: The Science And Practice Of Pharmacy”, Pharmaceutical Press, Easton, PA, 22^{nd} Edition, (2013) (hereafter, Remington 2013), the contents of which are incorporated herein by reference in their entirety. The various materials listed herein, alone or in combination, can be incorporated into or used with the materials described in Remington '95 or Remington 2013. Any suitable techniques, carriers, and excipients can be used, including those understood in the art; e.g., as described in Remington '95 or Remington 2013.

In certain embodiments, a pharmaceutical composition comprises a suitable excipient, non-limiting examples of which include anti-adherents (e.g., magnesium stearate), a binder, fillers, monosaccharides, disaccharides, other carbohydrates (e.g., glucose, mannose or dextrin), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington '95 or Remington 2013. The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art. For clarification, the term “binding agent” as used herein does not refer to a “binder” that is used in certain pharmaceutical formulations. Although a pharmaceutical composition, in certain embodiments, may comprise a binding agent that specifically binds cMET as well as a binder.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent include those having a sulfhydryl group (e.g., a thiol) such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOLR). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or binding agent-drug conjugate is substantially free of contaminants (e.g., blood cells, platelets, polypeptides, minerals, blood-borne compounds or chemicals, virus, bacteria, other pathogens, toxin, and the like). In some embodiments a composition, pharmaceutical composition or binding agent-drug conjugate is substantially free of serum and serum contaminants (e.g., serum proteins, serum lipids, serum carbohydrates, serum antigens and the like). In some embodiments a composition, pharmaceutical composition or binding agent-drug conjugate is substantially free of a pathogen (e.g., a virus, parasite or bacteria). In some embodiments a composition, pharmaceutical composition or binding agent-drug conjugate is substantially free of endotoxin. In some embodiments a composition, pharmaceutical composition or binding agent-drug conjugate is sterile. In certain embodiments, a composition or pharmaceutical composition comprises a binding agent-drug conjugate that specifically binds an extracellular domain of cMET and a suitable diluent (e.g., phosphate buffered saline).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parenteral administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parenteral administration (e.g., intravenous administration) to a mammal.

In certain embodiments, a pharmaceutical composition is configured for oral administration and may be formulated as a tablet, microtablet, minitablets, micropellets, powders granules, capsules (e.g., capsules filled with microtablets, micropellets, powders or granules), emulsions or solutions. Pharmaceutical compositions configured for oral administration may comprise suitable coatings to delay or sustain release of the active ingredient (e.g., a binding agent), non-limiting examples of which include enteric coatings such as fatty acids, waxes, shellac, plastics, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, sodium alginate, zein, plant fibers, the like and combinations thereof.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a binding agent from a topical patch.

In certain embodiments, an optimal pharmaceutical composition will be determined by one skilled in the art depending upon, for example, the intended route of administration, delivery format and desired dosage (see e.g., Remington '95 or Remington 2013, supra). In certain embodiments, such compositions may influence the physical state, stability, rate of in vivo release and rate of in vivo clearance of the antibody drug conjugates of the invention. A pharmaceutical composition can be manufactured by any suitable manner, including, e.g., by means of conventional mixing, dissolving, granulating, dragee-making, levigating, emulsifying, encapsulating, entrapping or tableting processes (e.g., see methods described in Remington '95 or Remington 2013).

### Second Medical Use

In some embodiments, presented herein is a composition or pharmaceutical composition for use as a medicament for the treatment of cancer or a neoplastic disorder in a subject, wherein the composition or pharmaceutical composition comprises a binding agent-drug conjugate described herein. In some embodiments, presented herein is a composition or pharmaceutical composition comprising a binding agent-drug conjugate described herein for use in the treatment of cancer or a neoplastic disorder.

### Methods of Treatment

In some embodiments a composition, pharmaceutical composition or binding agent-drug conjugate described herein is used to treat a subject having or suspected of having a neoplastic disorder or cancer. In certain embodiments, a binding agent-drug conjugate or pharmaceutical composition described herein is used in treating a neoplastic disorder or cancer in a subject, wherein the binding agent-drug conjugate specifically binds to an extracellular domain of human cMET. In some embodiments, presented herein is a method of treating a subject having or suspected of having a neoplastic disorder or cancer. In certain embodiments, a method of treating a subject having or suspected of having a neoplastic disorder or cancer comprises administering a therapeutically effective amount of a composition, pharmaceutical composition or binding agent-drug conjugate described herein to the subject. In certain embodiments, a method comprises contacting a cell (e.g., one or more cells) of a subject with a therapeutically effective amount of a composition, pharmaceutical composition or binding agent-drug conjugate described herein. In certain embodiments, a method comprises contacting a cancer cell or neoplastic cell of a subject with a therapeutically effective amount of a composition, pharmaceutical composition or binding agent-drug conjugate described herein. In certain embodiments, a method comprises contacting a cell (e.g., one or more cells) of a subject with a therapeutically effective amount of a binding agent-drug conjugate that specifically binds to an extracellular portion of human cMET, or variant thereof. In certain embodiments, a method comprises contacting a cancer cell or neoplastic cell with a therapeutically effective amount of a binding agent-drug conjugate that specifically binds to an extracellular portion of human cMET, or variant thereof, wherein the cell expresses cMET on its cell surface. The cell of a subject is often a cell that expresses an extracellular portion of cMET. A cell that is contacted with a binding agent-drug conjugate may be found inside a subject (e.g., in vivo) or outside a subject (e.g., in vitro or ex vivo).

In certain embodiments, a binding agent-drug conjugate blocks, inhibits, ameliorates, abrogates, or suppresses growth, viability or metastasis of a cancer or cancer cell. In certain embodiments, a binding agent-drug conjugate induces death, necrosis or apoptosis of a cancer or cancer cell. In certain embodiments, contacting a cell of a subject with a binding agent-drug conjugate disclosed herein induces or promotes death, necrosis or apoptosis of the cell. In certain embodiments, contacting a cell of a subject with a binding agent-drug conjugate disclosed herein induces or promotes death of a cell by an ADCC, ADCP or complement-dependent cellular cytotoxicity (CDCC) process. In certain embodiments, contacting a cell of a subject with a binding agent-drug conjugate disclosed herein decreases, inhibits, or reduces mitosis of the cell. In certain embodiments, contacting a cancer or cancer cell of a subject with a binding agent-drug conjugate disclosed herein decreases, inhibits, or reduces metastasis of the cancer or cancer cell.

### Subjects

The term “subject” refers to a mammal. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female.

In some embodiments a subject is in need of a treatment or composition described herein. In certain embodiments a subject has or is suspected of having a neoplastic disorder or a cancer. In some embodiments a subject in need of a treatment or composition described herein has or is suspected of having a neoplastic disorder or a cancer. In certain embodiments a binding agent-drug conjugate or composition described herein is used to treat a subject having, or suspected of having, a neoplastic disorder or cancer.

### Cancer Types

A composition, pharmaceutical composition or binding agent-drug conjugate disclosed herein can be used to treat a neoplastic order or cancer non-limiting examples of which include a carcinoma, sarcoma, neuro neoplasia, lymphoma, myeloma, leukemia, melanoma, mesothelioma, solid or soft tissue tumors, and secondary cancers (e.g., derived from a primary site)). Non-limiting examples of a carcinoma include respiratory system carcinomas, gastrointestinal system carcinomas, genitourinary system carcinomas, testicular carcinomas, prostatic carcinomas, endocrine system carcinomas, basal cell carcinoma of the skin, carcinoma of unknown primary, cholangiocarcinoma, ductal carcinoma in situ (DCIS), Merkel cell carcinoma, lung carcinoma, thymoma and thymic carcinoma, midline tract carcinoma, lung small cell carcinoma, thyroid carcinoma, liver hepatocellular carcinoma, squamous cell carcinoma, head and neck squamous carcinoma, breast carcinoma, epithelial carcinoma, adrenocortical carcinoma, ovarian surface epithelial carcinoma, and the like, further including carcinomas of the uterus, cervix, colon, pancreas, kidney, esophagus, stomach and ovary. Non-limiting examples of a sarcoma include Ewing sarcoma, lymphosarcoma, liposarcoma, osteosarcoma, breast sarcoma, soft tissue sarcoma, Kaposi sarcoma, rhabdomyosarcoma, uterine sarcoma, chondrosarcoma, leiomyosarcoma, fibrosarcoma and the like. Non-limiting examples of a neuro neoplasia include glioma, glioblastoma, meningioma, neuroblastoma, retinoblastoma, astrocytoma, oligodendrocytoma and the like. Non-limiting examples of lymphomas, myelomas, and leukemia include acute and chronic lymphoblastic leukemia, myeloblastic leukemia, multiple myeloma, poorly differentiated acute leukemias (e.g., erythroblastic leukemia and acute megakaryoblastic leukemia), acute promyeloid leukemia (APML), acute myelogenous leukemia (AML), chronic myelogenous leukemia (CML), acute lymphoblastic leukemia (ALL), which includes B-lineage ALL and T-lineage ALL, chronic lymphocytic leukemia (CLL), prolymphocytic leukemia (PLL), hairy cell leukemia (HLL), Waldenstrom's macroglobulinemia (WM), non-Hodgkin lymphoma and variants, peripheral T-cell lymphomas, adult T-cell leukemia/lymphoma (ATL), cutaneous T-cell lymphoma (CTCL), large granular lymphocytic leukemia (LGF), Hodgkin's disease and Reed-Sternberg disease. Non-limiting examples of soft or solid tissue tumors include visceral tumors, seminomas, hepatomas, and other tumors of the breast, liver, lung, pancreas, uterus, ovary, testicle, head, neck, eye, brain, mouth, pharynx, vocal cord, ear, nose, esophagus, stomach, intestine, colon, adrenal gland, kidney, bone, bladder, urethra, carcinomas, lung, muscle, skin, feet, hands, and soft tissue. In some embodiments, a neoplastic disorder or cancer that can be treated by a pharmaceutical composition or binding agent-drug conjugate disclosed herein is selected from a bladder cancer, breast cancer, colorectal cancer, cervical cancer, gastric cancer, liver cancer, hepatocellular cancer, hypopharynx cancer, lung cancer, adenocarcinoma, ovarian cancer and renal cancer. In some embodiments, a neoplastic disorder or cancer that can be treated by a pharmaceutical composition or binding agent-drug conjugate disclosed herein is selected from a pancreatic cancer (e.g., a pancreatic adenocarcinoma, exocrine pancreatic cancer or pancreatic neuroendocrine cancer), a colorectal cancer (e.g., a colorectal adenocarcinoma), small intestinal malignancy, cholangiocarcinoma, non-small cell lung cancer (NSCLC), thyroid carcinoma, esophageal or esophagogastric junction (EGJ) cancer, gastric adenocarcinoma, liver hepatocellular carcinoma, head and neck squamous carcinoma, female genital tract malignancy, breast carcinoma, lung small cell carcinoma, ovarian surface epithelial carcinoma, retroperitoneal or peritoneal sarcoma, prostatic adenocarcinoma, neuroendocrine tumor, gastrointestinal stromal tumor, glioblastoma or non-epithelial ovarian cancer. In some embodiments, a neoplastic disorder or cancer that can be treated by a pharmaceutical composition or binding agent-drug conjugate disclosed herein is a breast cancer, non-limiting examples of which include ductal carcinoma in situ (DCIS), invasive ductal carcinoma (IDC) (e.g., tubular carcinoma of the breast, medullary carcinoma of the breast, mucinous carcinoma of the breast, papillary carcinoma of the breast, and cribriform carcinoma of the breast), invasive lobular carcinoma (ILC), inflammatory breast cancer, lobular carcinoma in situ (LCIS), male breast cancer, molecular subtypes of breast cancer (e.g., Luminal B breast cancer or hormone-receptor positive breast cancer, Triple-negative breast cancer, HER2-enriched breast cancer, and normal-like breast cancer), Paget's disease of the nipple, phyllodes tumors of the breast, and metastatic breast cancer. In some embodiments a neoplastic disorder or cancer that can be treated by a pharmaceutical composition or binding agent-drug conjugate disclosed herein is a triple negative breast cancer.

In some embodiments, the effectiveness of a treatment described herein can be determined or predicted, in part, by an amount of cMET that a cancer or neoplasia expresses. Many cancer and tumor types are known to express cMET, non-limiting examples of which include certain bladder cancers, breast cancers, colorectal cancers, gastric cancers, hepatocellular cancers, HNSCC, hypopharynx cancers, lung cancers, adenocarcinomas, ovarian cancers and renal cancers (e.g., see Ariyawutyakorn et al. (2016) Journal of Cancer 7 (6): 633-649) and the amounts of cMET expressed by many cancer types are known (e.g., see Arguello et al. (2013) Annual Meeting of Association for Molecular Pathology (AMP) Abstract No. 294319). In addition, a neoplastic cell or cancer cell can be quickly assayed for expression of cMET using a suitable anti-cMET binding agent (e.g., antibody) using a suitable method (e.g., whole-cell ELISA, FACs, any suitable immunoassay, and the like). Accordingly, in some embodiments, a method of treating a subject having or suspected of having a cancer comprises administering a therapeutically effective amount of a binding agent-drug conjugate described herein, or a pharmaceutical composition comprising a binding agent-drug conjugate described herein, to the subject, wherein the cancer expresses detectable levels of cMET. In certain embodiments, a cancer that expresses detectable levels of cMET can be a cancer that is known or reported to express cMET, or is suspected of expressing cMET (e.g., by having a similar genotype or phenotype to another cancer that is known to express cMET). In some embodiments, a cancer that expresses cMET, or a cancer that is suspected of expressing cMET is a cancer that expresses an RNA transcript that encodes cMET, or a portion thereof. In some embodiments, a cancer that expresses cMET or a cancer that is suspected of expressing cMET is a cancer that expresses cMET on its cell surface.

### Route of Administration

Any suitable method of administering a composition, pharmaceutical composition or binding agent-drug conjugate to a subject can be used. The exact formulation and route of administration for a composition for use according to the methods of the invention described herein can be chosen by a medical professional (e.g., a physician) in view of a patient's condition. (e.g., see Fingl et al. 1975, in “The Pharmacological Basis of Therapeutics”, which is incorporated herein by reference in its entirety). Any suitable route of administration can be used for administration of a pharmaceutical composition or a binding agent-drug conjugate described herein. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is sometimes provided to a subject for self-administration or for administration to a subject by another (e.g., a non-medical professional). For example a composition described herein can be provided as an instruction written by a medical practitioner that authorizes a patient to be provided a composition or treatment described herein (e.g., a prescription). In another example, a composition can be provided to a subject where the subject self-administers a composition orally, intravenously or by way of an inhaler, for example.

Alternately, one can administer compositions for use according to the methods of the invention in a local rather than systemic manner, for example, via direct application to the skin, mucous membrane or region of interest for treating, including using a depot or sustained release formulation.

In some embodiments a pharmaceutical composition comprising a binding agent-drug conjugate can be administered alone (e.g., as a single active ingredient (AI or e.g., as a single active pharmaceutical ingredient (API)). In other embodiments, a pharmaceutical composition comprising a binding agent-drug conjugate can be administered in combination with one or more additional AIs/APIs, for example, as two separate compositions or as a single composition where the one or more additional AIs/APIs are mixed or formulated together with the binding agent-drug conjugate in a pharmaceutical composition.

In certain embodiments, a cMET binding agent-drug conjugate is delivered to a cell (e.g., a mammalian cell). A cMET binding agent-drug conjugate can be delivered to a cell using any suitable method. In certain embodiments, delivering a cMET binding agent-drug conjugate to a cell comprises contacting a mammalian cell, in vitro or in vivo, with a composition comprising a cMET binding agent-drug conjugate under conditions that allow the binding agent-drug conjugate to bind to the cell.

### Dose and Therapeutically Effective Amount

In some embodiments, an amount of a binding agent-drug conjugate in a composition is a therapeutically effective amount. In some embodiments, a therapeutically effective amount of a binding agent-drug conjugate is administered to a subject. In some embodiments, an therapeutically effective amount of a binding agent-drug conjugate in a composition is an amount needed to obtain an effective therapeutic outcome. In certain embodiments, the amount of a binding agent-drug conjugate in a composition (e.g., a pharmaceutical composition) is an amount sufficient to prevent, treat, reduce the severity of, delay the onset of, and/or alleviate a symptom of a neoplastic disorder or cancer, as contemplated herein.

A “therapeutically effective amount” means an amount sufficient to obtain an effective therapeutic outcome and/or an amount sufficient to prevent, treat, reduce the severity of, delay the onset of, and/or alleviate a symptom of a neoplastic disorder or cancer. In certain embodiments, a “therapeutically effective amount” means an amount sufficient to terminate the growth of, and/or slow the growth of a neoplasm or cancer. In certain embodiments, a “therapeutically effective amount” means an amount sufficient to inhibit the replication of, and/or induce the death of one or more neoplastic or cancer cells. Determination of a therapeutically effective amount is well within the capability of those skilled in the art, especially in light of the detailed disclosure provided herein.

In certain embodiments, a therapeutically effective amount is an amount high enough to provide an effective therapeutic effect and an amount low enough to minimize unwanted adverse reactions. Accordingly, in certain embodiments, a therapeutically effective amount of a binding agent-drug conjugate may vary from subject to subject, often depending on age, weight, general health condition of a subject, severity of a condition being treated, and a particular combination of drugs administered. Thus, in some embodiments, a therapeutically effective amount is determined empirically. Accordingly, a therapeutically effective amount of a binding agent-drug conjugate used to treat a subject can be determined by one of ordinary skill in the art based on amounts found effective in animal or clinical studies, a physician's experience, and suggested dose ranges or dosing guidelines, for example.

In certain embodiments, a binding agent-drug conjugate (e.g., a binding agent-drug conjugate in a pharmaceutical composition) is administered at a suitable therapeutically effective amount or dose (e.g., at a suitable volume and concentration, which sometimes depends, in part, on a particular route of administration). In certain embodiments, a therapeutically effective amount of a binding agent-drug conjugate is selected from one or more doses of about 0.01 mg/kg (e.g., per kg body weight of a subject) to 500 mg/kg, 0.1 mg/kg to 500 mg/kg, 0.1 mg/kg to 400 mg/kg, 0.01 mg/kg to 300 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg, 0.1 mg/kg to 1 mg/kg, intervening amounts and combinations thereof. In some aspects the therapeutically effective amount of a binding agent-drug conjugate comprises one or more doses of about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, and 0.1 mg/kg, intervening amounts and combinations thereof. In some embodiments a therapeutically effective amount of a binding agent-drug conjugate is between about 0.1 mg/kg to 100 mg/kg, or between about 1 mg/kg and about 50 mg/kg.

In some embodiments administering a therapeutically effective amount of a binding agent-drug conjugate or a pharmaceutical composition comprising a binding agent-drug conjugate comprises administering a suitable dose at a frequency or interval as needed to obtain an effective therapeutic outcome. In some embodiments administering a therapeutically effective amount of a binding agent-drug conjugate or a pharmaceutical composition comprising a binding agent-drug conjugate comprises administering a suitable dose hourly, every two hours, every 4 hours, every 6 hours, three times a day, twice a day, once a day, six times a week, five times a week, four times a week, three times a week, twice a week, weekly, at combinations thereof, and/or at regular or irregular intervals thereof, and/or simply at a frequency or interval as needed or recommended by a medical professional. An effective therapeutic outcome can be determined, in certain embodiments, by monitoring the number, size, viability, growth, mitosis, or metastasis of a cancer, neoplastic growth or cancerous cells in a subject. Accordingly, in certain embodiments, a decrease or reduction in the number, viability, size, growth, mitosis, or metastasis of neoplastic or cancerous cells in a subject is considered an effective therapeutic outcome.

### Kits

A pharmaceutical composition comprising an amount or dose of a binding agent-drug conjugate can, if desired, be provided in a kit, pack or dispensing device, which can contain one or more doses of a binding agent. In some embodiments, a kit comprises a pack and/or dispensing device. Non-limiting examples of a pack include a metal, glass, or plastic container, or blister pack that comprises a binding agent-drug conjugate or composition described herein. In certain embodiments, a kit comprises a dispensing device such as a syringe or inhaler. A pack and/or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a binding agent-drug conjugate sufficient to treat a patient for 1 day to 1 year, 1 day to 180 days, 1 day to 120 days, 1 day to 90 days, 1 day to 60 days, 1 day to 30 days, 1-24 hours, 1-12 hours, 1-4 hours, or amount of time there between.

A kit optionally includes a product label and/or one or more packaging inserts, that provide a description of the components or instructions for use in vitro, in vivo, or ex vivo, of the components therein. Exemplary instructions include instructions for a diagnostic method, treatment protocol or therapeutic regimen. In certain embodiments, a kit comprises packaging material, which refers to a physical structure housing components of the kit. The packaging material can maintain the components sterilely, and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory-type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein. Product labels or inserts can include information on potential adverse side effects and/or warnings.

### EXAMPLES

### Example 1—Antibody Generation

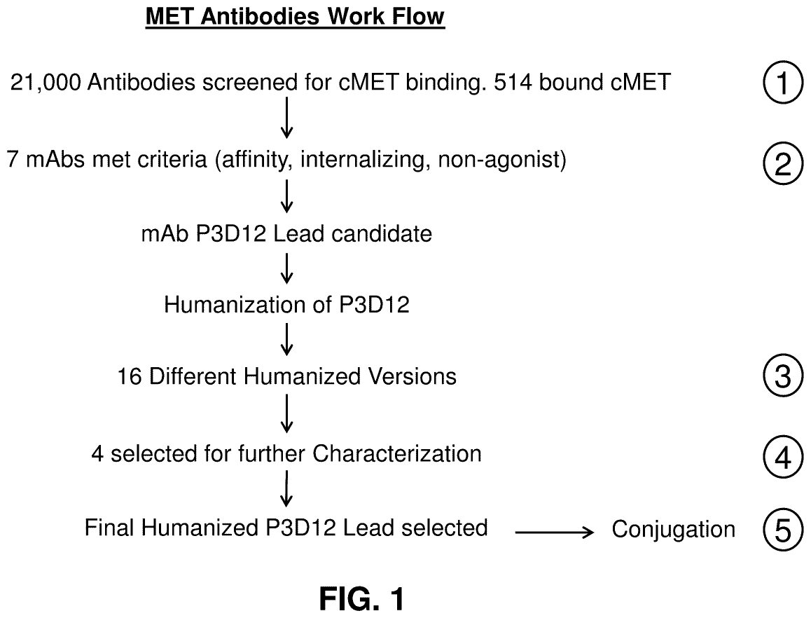

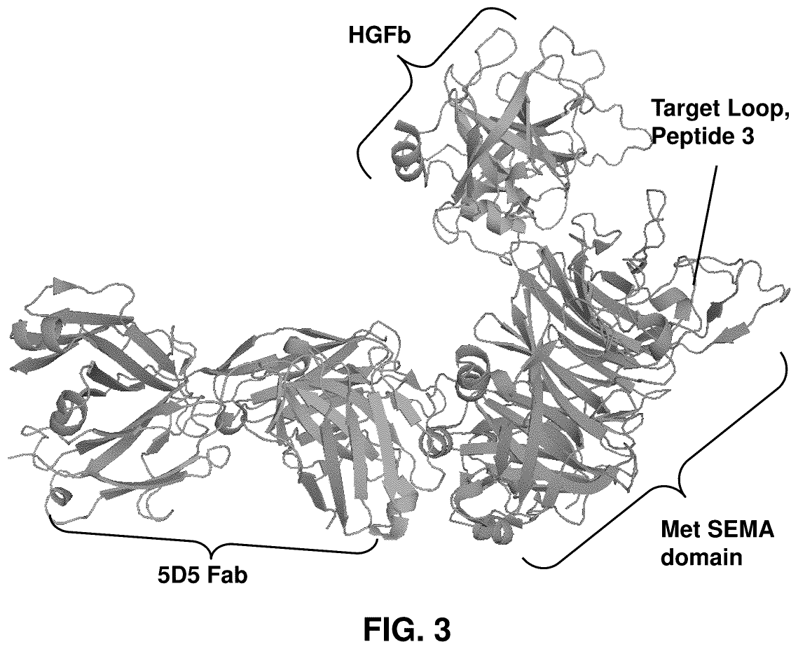

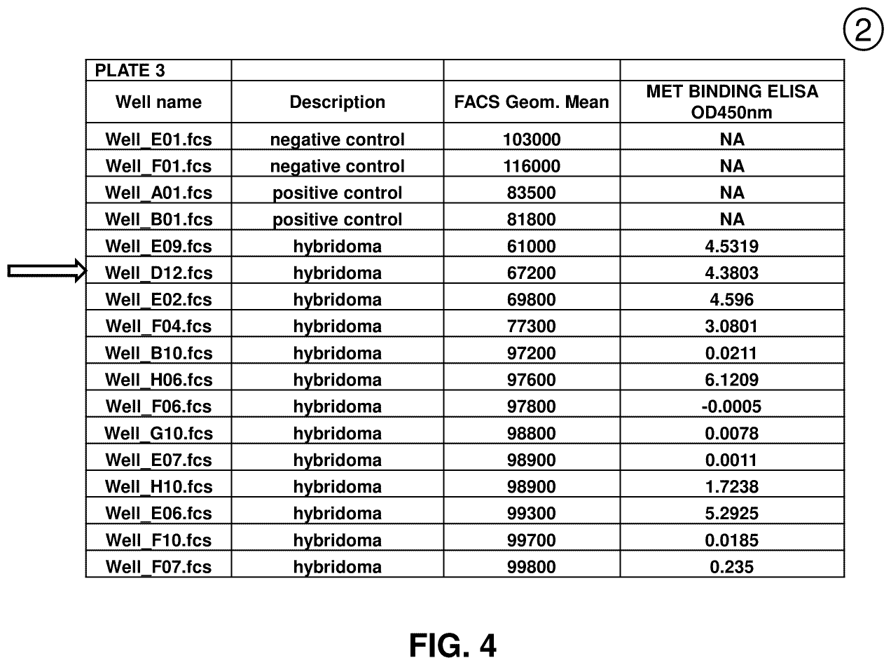

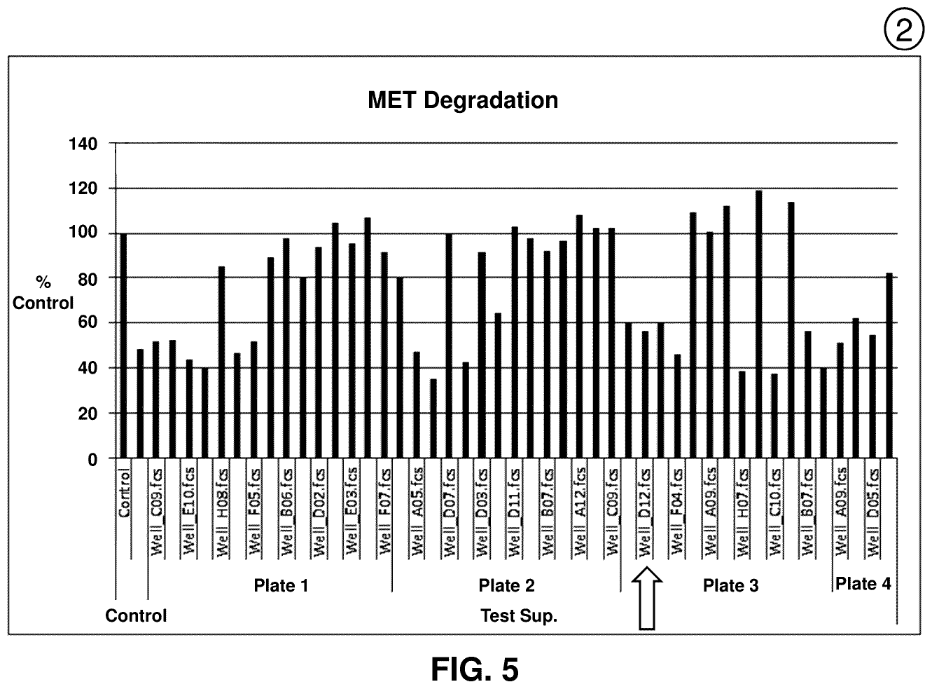

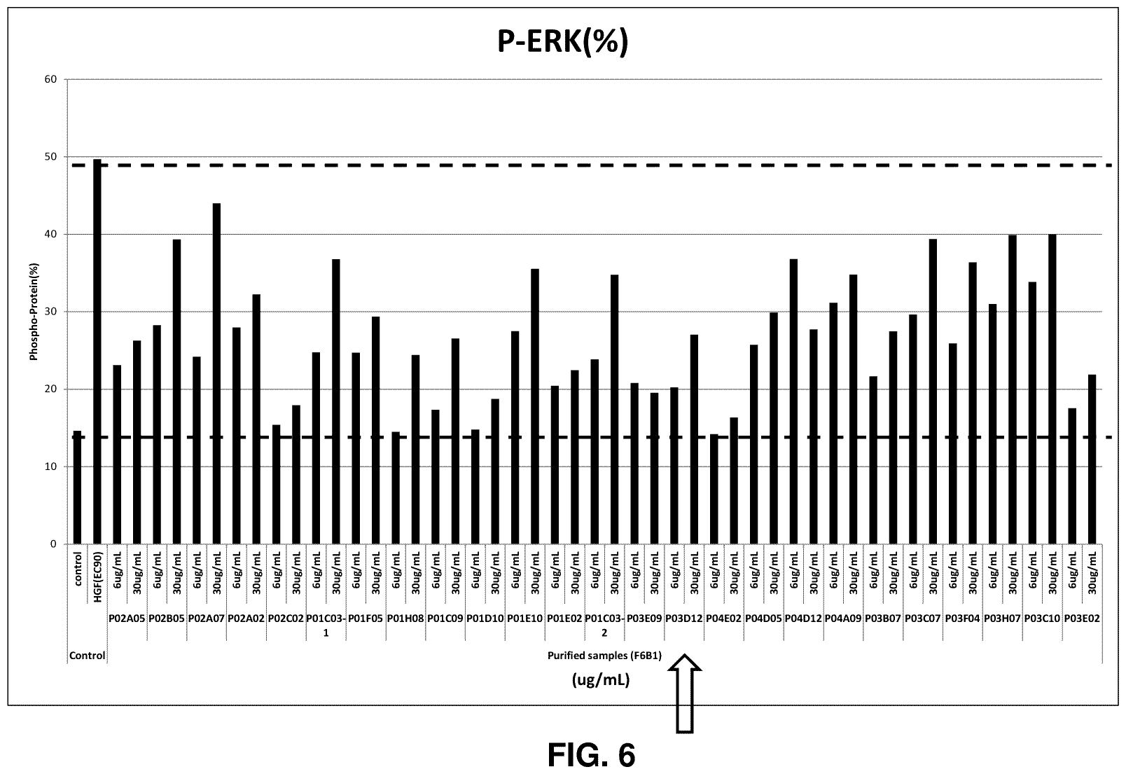

To induce an antibody response against cMET, mice were immunized with cMET-Fc or cMET peptide as described in FIGS. 1 and 2. In some embodiments peptide derived from strategic regions were selected for immunization. FIG. 3 illustrates an example of a structural loop on MET that inspired the design of peptide 3. Spleens from immunized mice were obtained and splenocytes were fused to a suitable fusion partner to produce hybridomas using a standard protocol. Hybridoma clones were isolated and hybridoma media was tested for binding to MET and/or for the ability to induce internalization of cMET on human cancer cell lines as measured by flow cytometry (FIG. 4). Selected hybridoma antibodies were selected for their ability to induce MET degradation (FIG. 5) or inability to induce phosphorylation of ERK (FIG. 6). The lead hybridoma F6B1P3D12 has been deposited with American Type Culture Collection, Patent Depository of 10801 University Boulevard Manassas, Virginia 20110-2209 USA on Mar. 20, 2019 under ATCC PTA-125826. The deposit was made under the provisions of the Budapest Treaty on the International Recognition of the Deposit of Microorganisms for the Purpose of Patent Procedure and the Regulations thereunder (Budapest Treaty.

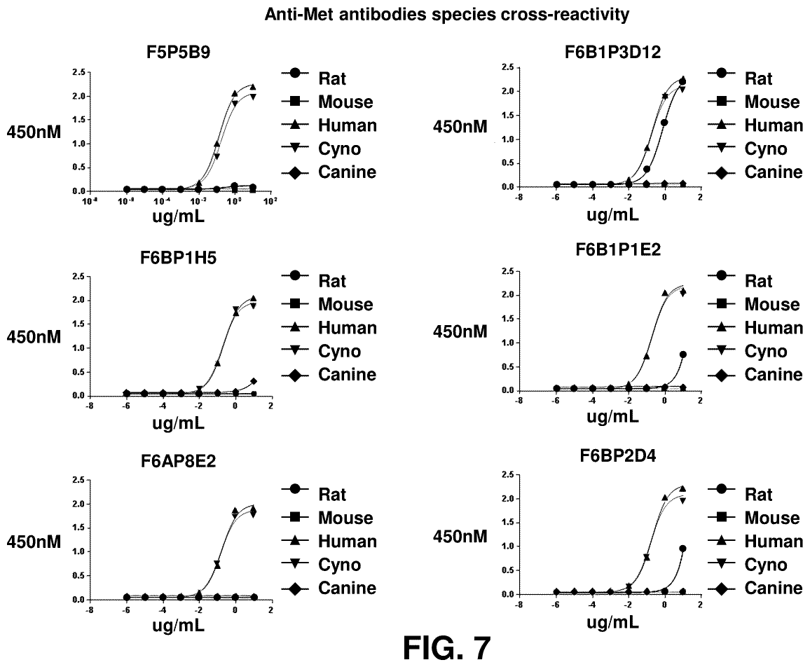

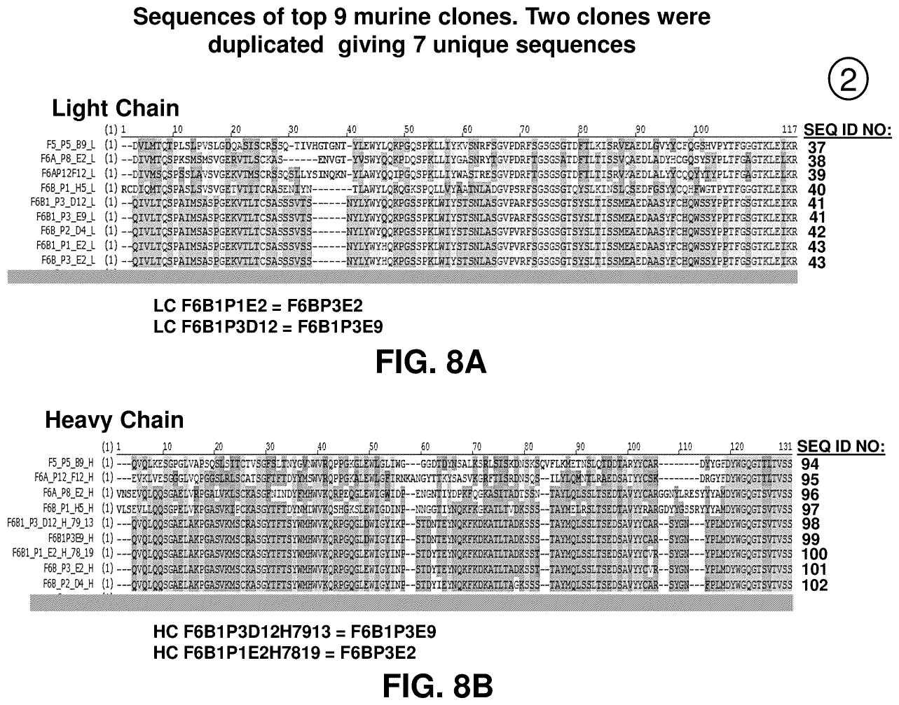

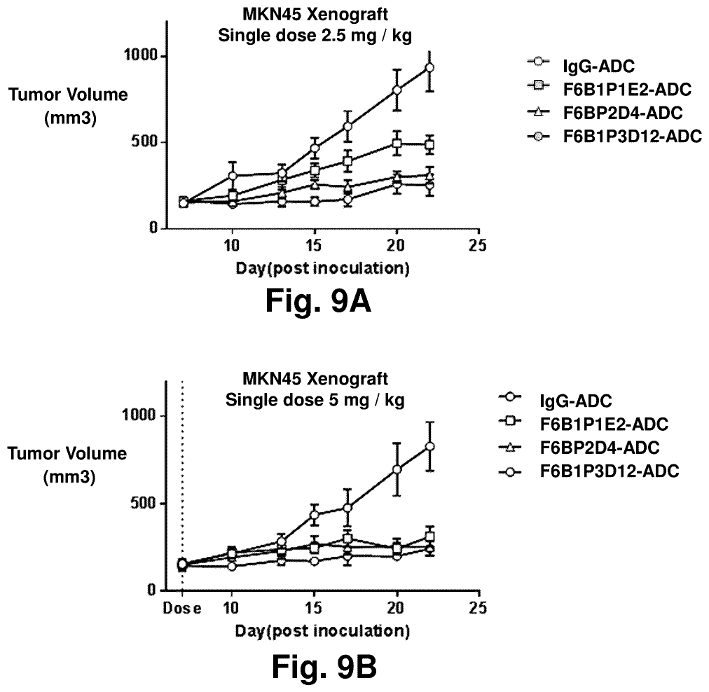

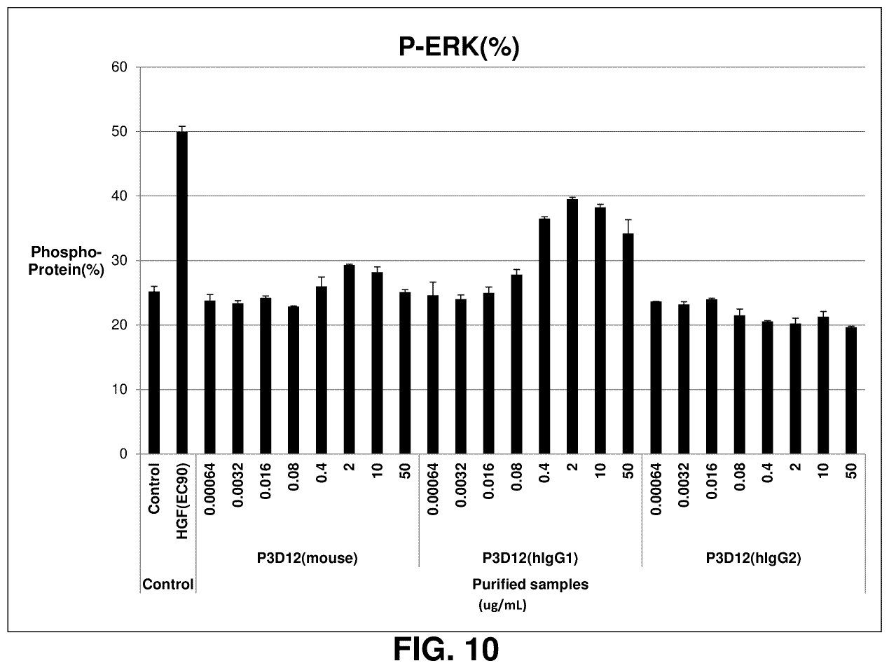

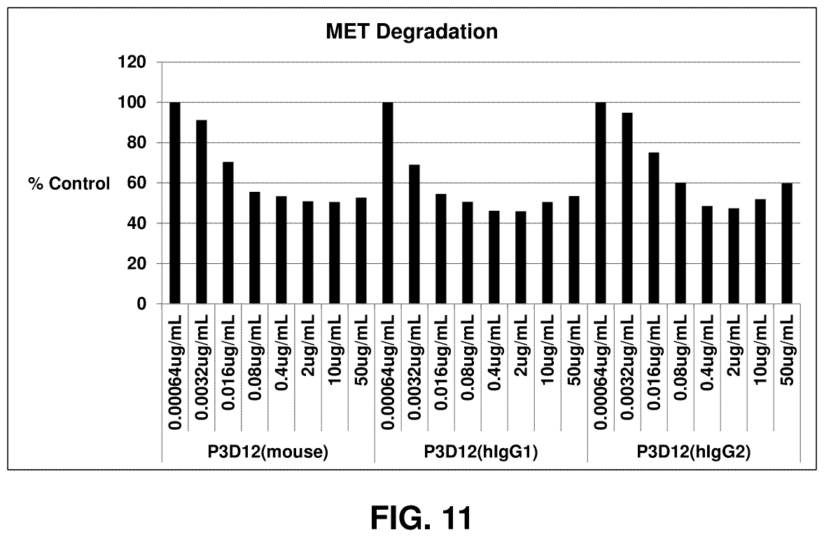

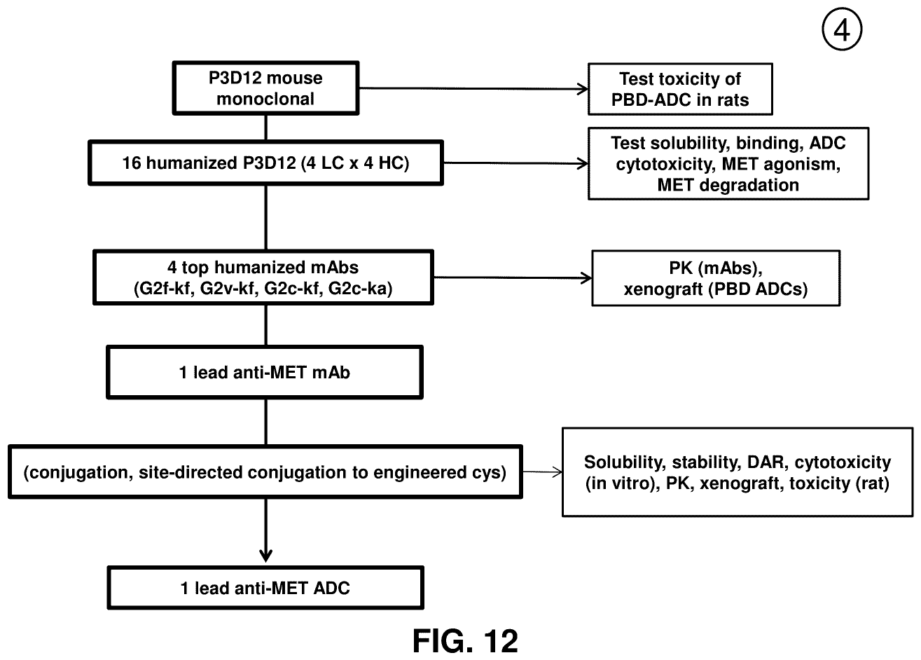

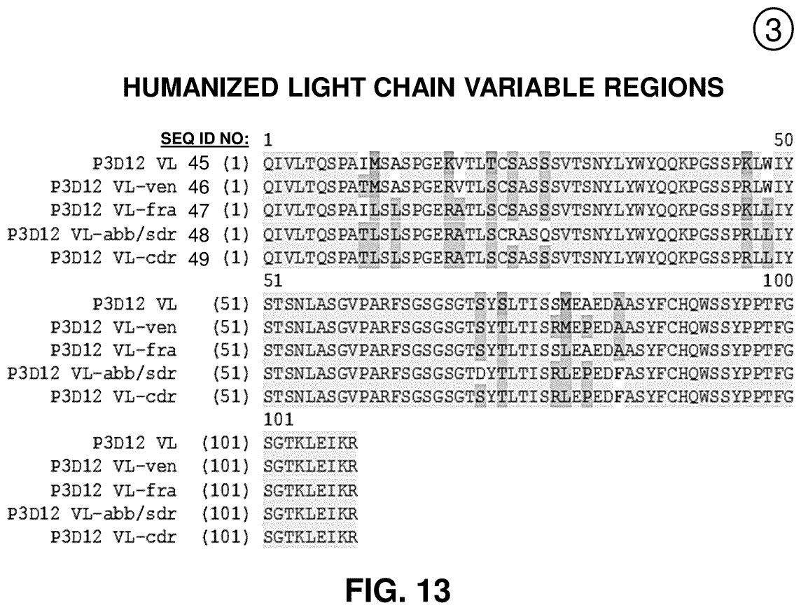

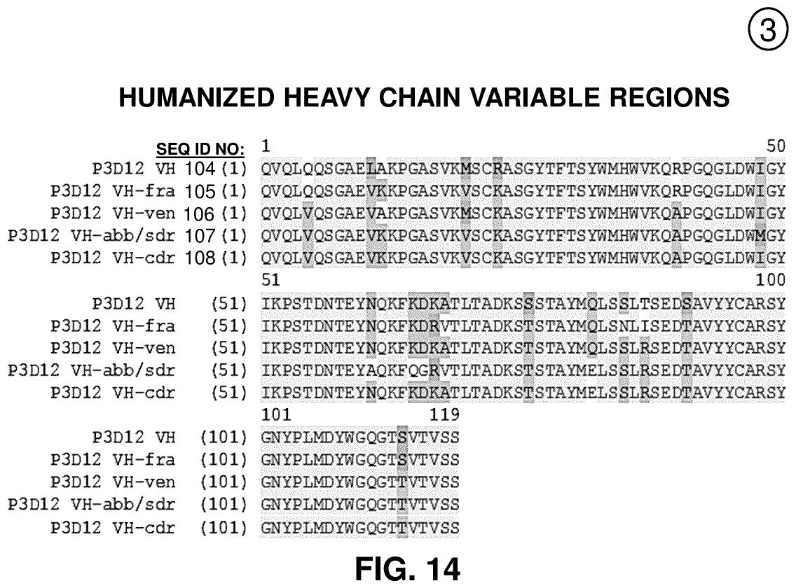

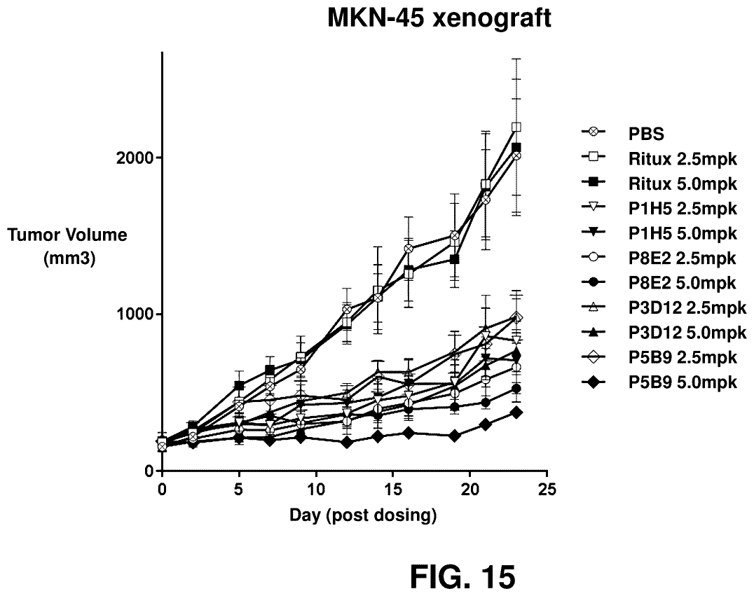

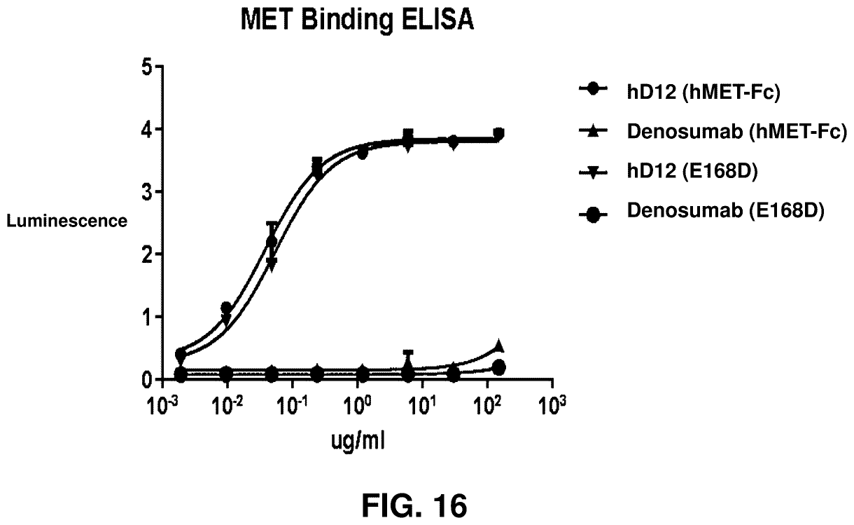

Additional assays were performed to select ideal anti-cMET antibody candidates. For example, anti-cMET antibodies were tested for species cross-reactivity by determining the ability of an antibody to bind to human cMET, monkey cMET (e.g., Macaca fascicularis, i.e., Cynomolgus Macaque), rat cMET and mouse cMET as measured by ELISA (FIGS. 7 & Table 11). In vivo half-life and other pharmacokinetic characteristics were also evaluated (data not shown). Potency and specificity of antibody drug conjugates (ADC) was also determined on high, medium and negative cMET expressing cell lines using anti-cMET antibodies that were conjugated to MMAF) (FIGS. 9 and 15, Tables 11 and 12). ADCs were tested for efficacy in vivo using an MKN45 Xenograft model.

TABLE 11

| | | Avg. | Avg. | | | | | | |
|---|---|---|---|---|---|---|---|---|---|
| | | Cytotoxicity | Cytotoxicity | | | Cell | | | |
| | | EC50, pM | EC50, pM | Met Binding | Met | Proliferation | | | |
| | | SNU16 | SNU20 | KD (pM) | Degradation | (Agonism) | | | |
| In vitro | | cells (Met | cells (Met | SPR | (pM) | ERK Phospho | Cross-reactivity | | |
| Ranking | Antibody | medium) | high) | w/hMet-Fc | MSD | Assay | NHP | Rat | Mouse |
| | 5D5 | | | 800 | | Yes/Strong | Yes | No | No |
| | ABF46 | 450 | 67 | 700 | 10,000 | NO | Yes | No | No |
| 1 | F6B1P3D12 | 25 | 60 | 750 | 140 | NO | Yes | Yes | No |
| 2 | F6BP2D4 | 60 | 33 | 480 | 3,000 | NO | Yes | No | No |
| 2 | F6B1P1E2 | 70 | 55 | 890 | 1,920 | Very low | Yes | Low | No |
| 3 | F6AP12F12 | 180 | 97 | 110 | 450 | NO | Not tested | Not tested | Not tested |
| 3 | F5P5B9 | 20 | 3 | 80 | 255 | Very low | Yes | No | No |
| 3 | F6AP8E2 | 351 | 102 | 160 | 7,000 | Very low at high | Yes | No | No |
| | | | | | | concentrations | | | |
| 3 | F6BP1H5/H6 | 80 | 63 | 340 | 3,000 | Maybe at high | Yes | No | No |
| | | | | | | concentrations | | | |

*SPR = Surface Plasmon Resonance
**MSD = Meso Scale Discovery Platform
***NHP-Non-human Primate (i.e., Cynomolgus Macaque)
5D5 = agonist positive control
ABF46 = MET ADC, positive control

### Example 2—Summary of Characteristics of Selected Humanized Monoclonal Binding Agents

Humanized and isotype switched monoclonal binding agents were generated which comprise the heavy chain CDRs and light chain CDRs of the mouse monoclonal antibody P3D12. Sixteen different heavy chain (HC) and light chain (LC) combinations were tested for solubility in PBS, binding to human cMET, binding to rat cMET, binding affinity to human and rat cMET as determined by surface plasmon resonance (SPR), the presence of agonistic activity and cMET degradation reported as Meso Scale Discovery platform (MSD). The results are summarized in Table 12 below.

TABLE 12

| | | | | | | | | | vc-MMAF | | | | MET |
|---|---|---|---|---|---|---|---|---|---|---|---|---|---|
| | | | | | | | ELISA | ELISA | ADC | | | pERK | degradation |
| | | SEQ | | SEQ | | Solubility | hMET | rMET | IC50, pM, | SPR kD, | SPR kD, | (MET | equal to |
| | | ID | | ID | | in | vs | vs | SNU-16 | nM, | nM, | agonism, | parental? |
| Rank | HC | NO: | LC | NO: | Clone | PBS | parental | parental | (n = 1) | hMET-Fc | rMET-Fc | MSD) | (MSD) |
| | VH-abb/sdr | 107 | VL-abb/sdr | 48 | G2aka | | 0.9 | 1.1 | 352 | 0.9 | 26 | negative | yes |
| | | | VL-fra | 47 | G2akf | | 1.0 | 0.5 | 368 | 0.6 | 23 | negative | yes |
| | | | VL-ven | 46 | G2akv | insoluble | 1.6 | 1.3 | | | | negative | yes |
| | | | VL-cdr | 49 | G2akc | | 1.3 | 0.7 | 382 | 0.7 | 38 | negative | yes |
| | VH-fra | 105 | VL-abb/sdr | 48 | G2fka | | 1.8 | 3.8 | | | | negative | yes |
| 3 | | | VL-fra | 47 | G2fkf | | 1.7 | 1.4 | 266 | 0.4 | 30 | negative | yes |
| | | | VL-ven | 46 | G2fkv | insoluble | 1.7 | 1.2 | | | | negative | yes |
| | | | VL-cdr | 49 | G2fkc | | 1.7 | 2.3 | | | | negative | yes |
| | VH-ven | 106 | VL-abb/sdr | 48 | G2vka | | 0.9 | 2.7 | | | | negative | yes |
| 2 | | | VL-fra | 47 | G2vkf | | 1.1 | 1.0 | 380 | 0.3 | 8 | negative | yes |
| | | | VL-ven | 46 | G2vkv | insoluble | 1.6 | 1.6 | | | | negative | yes |
| | | | VL-cdr | 49 | G2vkc | | 1.1 | 1.9 | | | | negative | yes |
| 4 | VH-cdr | 108 | VL-abb/sdr | 48 | G2cka | | 1.3 | 2.7 | 238 | 0.4 | 29 | negative | yes |
| 1 | | | VL-fra | 47 | G2ckf | | 0.7 | 0.9 | 302 | 0.3 | 7 | negative | yes |
| | | | VL-ven | 46 | G2ckv | insoluble | 1.1 | 1.0 | | | | negative | yes |
| | | | VL-cdr | 49 | G2ckc | | 1.0 | 2.0 | 169 | 0.9 | 31 | negative | yes |
| | Chimeric | 104 | | 45 | | | | | 249 | 0.7 | 30 | | |
| | P3D12 | | | | | | | | | | | | |
| | Mouse | 98 | | 41 | | | | | 42 | 0.9 | 16 | | |
| | P3D12 | | | | | | | | | | | | |

Monoclonal humanized antibody of IgG2 isotype comprising a humanized light chain variable region of SEQ ID NO: 47 and a humanized heavy chain variable region of SEQ ID NO: 108 was selected as a representative humanized anti-cMET antibody, named hD12 and used as the antibody in the following examples.

### Example 3—Binding Assays

A representative humanized anti-cMET antibody, hD12 comprising the humanized heavy chain of SEQ ID NO: 105 and the humanized light chain sequences of SEQ ID NO: 47 was stochastically conjugated to five representative payloads (i.e., payloads of chemical formulas II, IV, VI, VII and XI), each payload comprising a pyrrolobenzodiazepine toxin and a linking group. In this example, the linking groups of the payload were stochastically linked to the hD12 antibody using disulfide chemistry. Briefly, the hD12 antibody was first subjected to reduction with glutathione (GSH), unreacted GSH was removed, and the linking group, which comprises a reactive maleimide group, is reacted with one or more free sulfhydryl groups (i.e., thiol groups) on the antibody. Using this approach, one or more payloads are covalently linked to the hD12 antibody at random positions occupied by a cysteine residue. Accordingly, this method is referred to a stochastic conjugation.

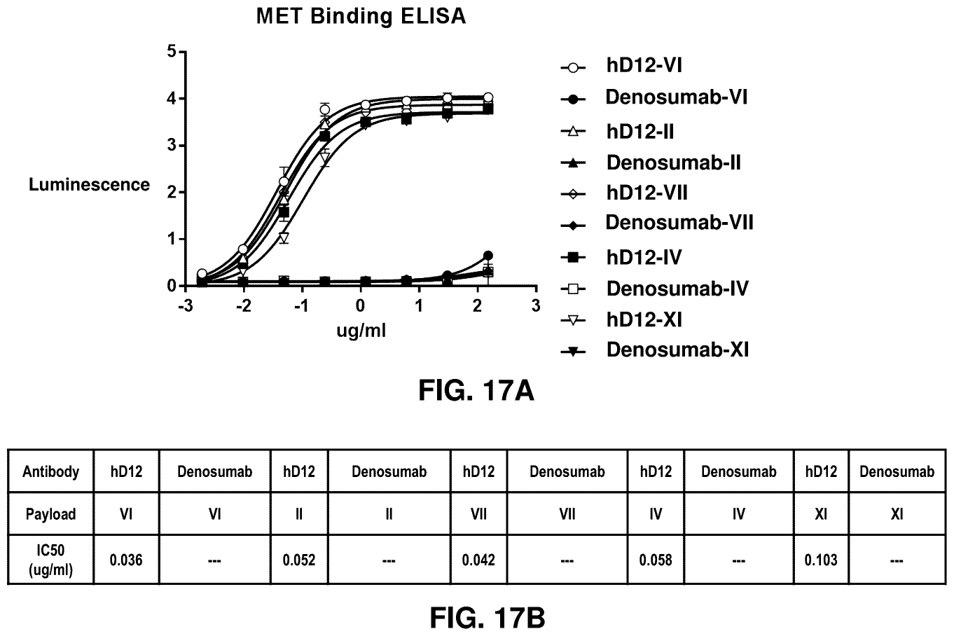

The resulting hD12 antibody drug conjugates (i.e., hD12-II (Antibody hD12 attached to the payload of chemical formula II); hD12-IV (Antibody hD12 attached to the payload of chemical formula IV); hD12-VI (Antibody hD12 attached to the payload of chemical formula VI); hD12-VII (Antibody hD 12 attached to the payload of chemical formula VII); and hD12-XI (Antibody hD12 attached to the payload of chemical formula XI)) were assayed for binding to plate-bound cMET by ELISA. Denosumab conjugated to each of the five payloads was used as a negative control, as the monoclonal antibody Denosumab specifically binds to RANK ligand (RANKL) and does not bind to cMET. The results of the ELISA binding study are shown in FIGS. 17A and 17B. There were no significant differences in binding between the 5 different hD12 drug conjugates to cMET. The isotype control conjugates of Denosumab did not bind to cMET as expected.

Materials: High binding 384 well plates (Thermo Fisher #: 8755), Blocking buffer (SkyTek Lab #AAA500), Recombinant Human c-Met-10X His (1.04 mg/ml, in-house, Lot #140924TA), Anti-human Kappa Light chain HRP conjugated (1 mg/ml, Brthyl #AP80-219P), 1x KPL wash buffer in water (20x, 200 ml, KPL #50-63-01), TMB (100 ml, KPL #53-00-00), and Stop solution (Cell Signaling #7002L).

### Example 4—Cytotoxicity Assays

The cytotoxicity of the five hD12 antibody drug conjugates of Example 3 were tested against cells expressing different levels of surface cMET. Denosumab conjugated to each of the five representative payloads was used as a negative control (Data not shown). Denosumab conjugates had little or no effect on cell killing in cMET expressing cell lines.

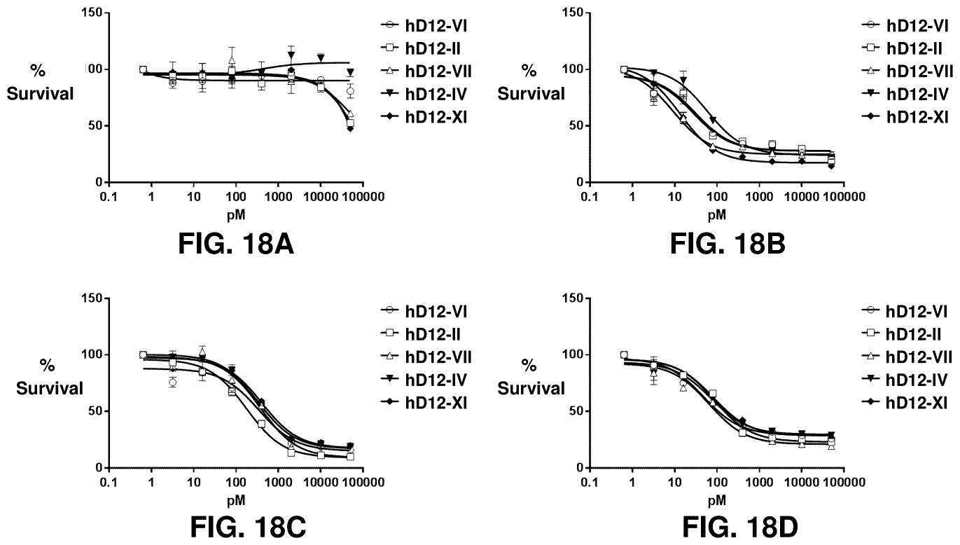

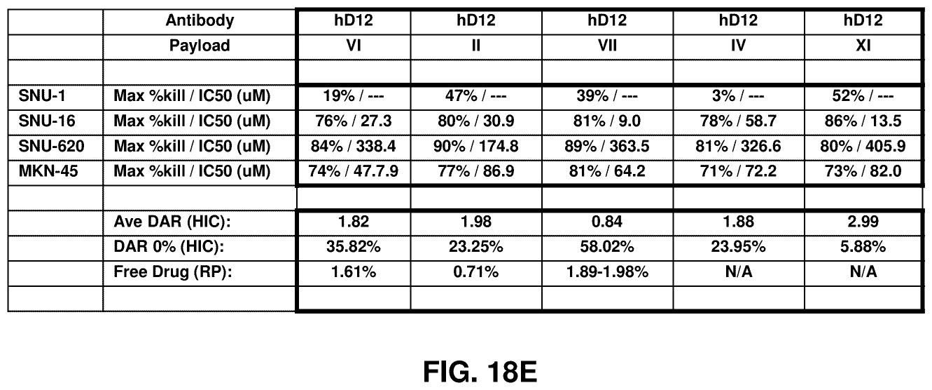

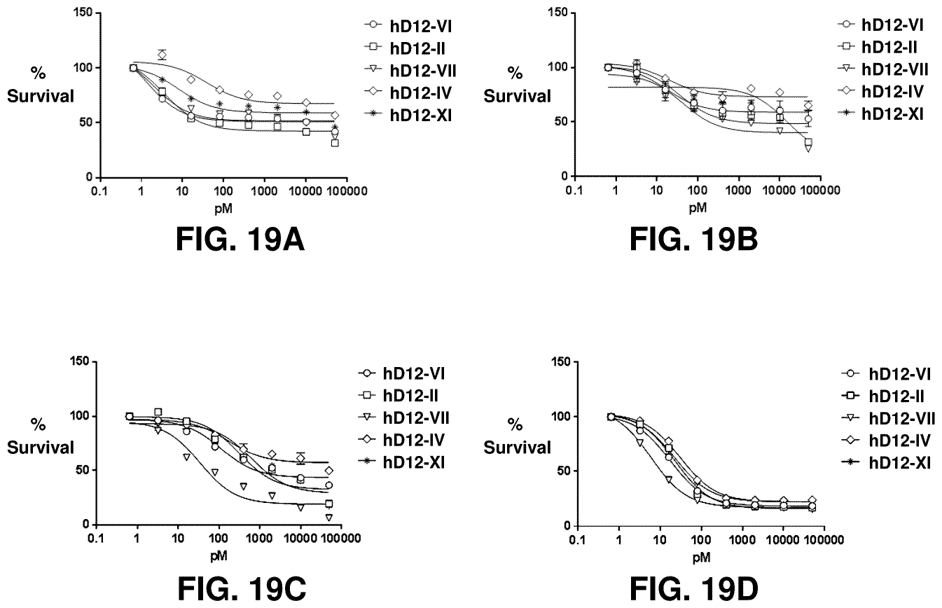

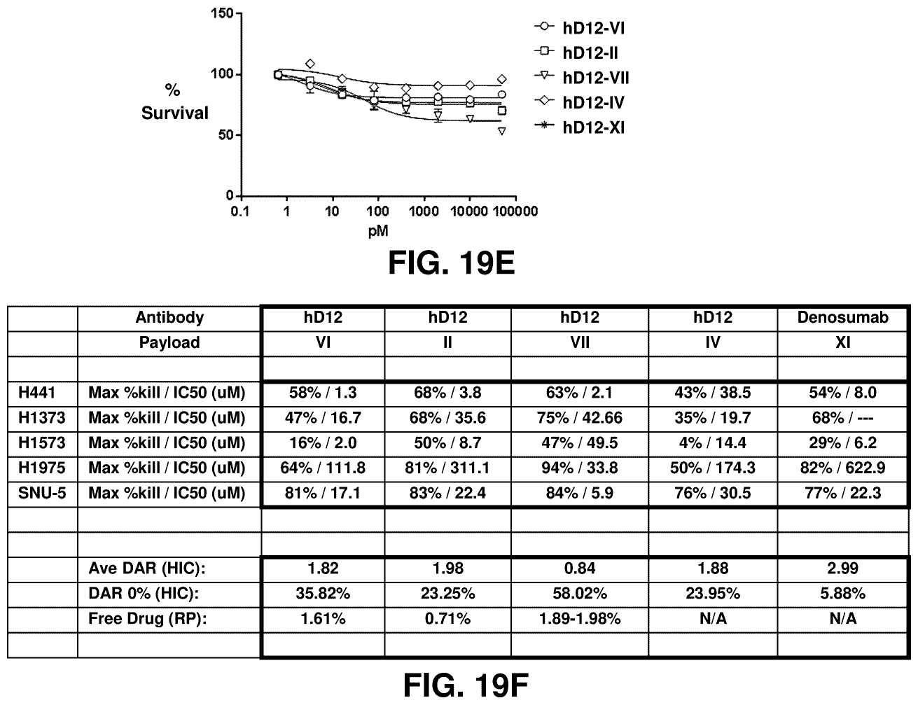

The results of the cytotoxicity assay are shown in FIGS. 18A-18E and FIGS. 19A-19F. The cell lines tested were SNU-1 (ATCC, no expression of cMET, FIG. 18A), SNU-16 (ATCC, medium expression of cMET, FIG. 18B), SNU-620 (KCLB, high expression of cMET, FIG. 18C), MKN-45 (DSMZ, high expression of cMET, FIG. 18D), H441 (ATCC, medium expression of cMET, FIG. 19A), H1373 (ATCC, medium expression of cMET, FIG. 19B), H1975 (ATCC, medium expression of cMET, FIG. 19C), SNU-5 (ATCC, high expression of cMET, FIG. 19D), and H1573 (ATCC, medium expression of cMET, FIG. 19E). hD12-II and hD12-VII showed slightly higher potency in some medium and high cMET-expressing cell lines.

### Example 5—Xenograft Studies

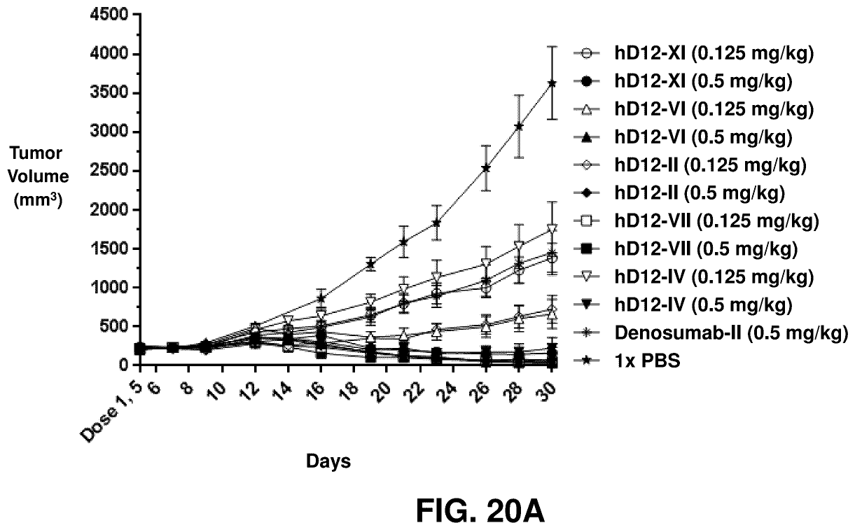

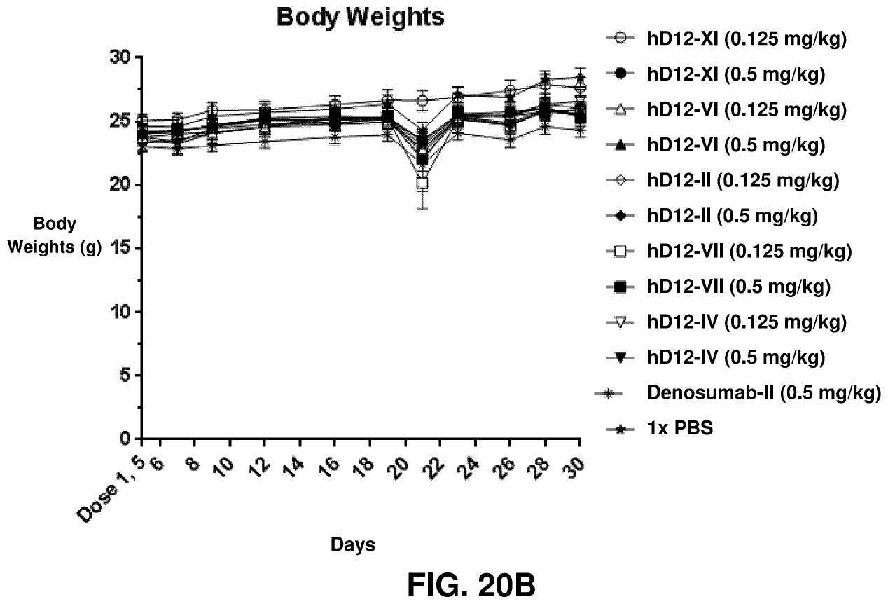

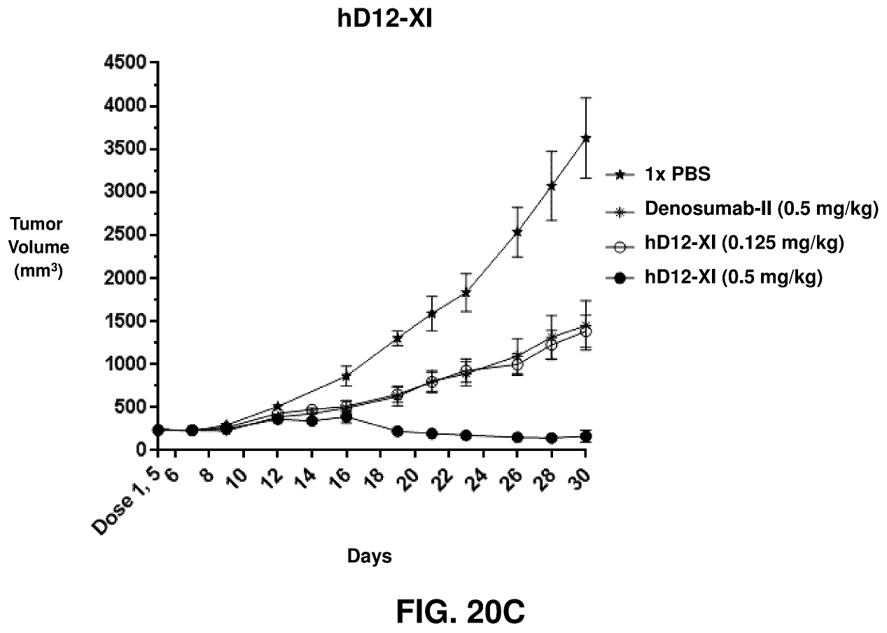

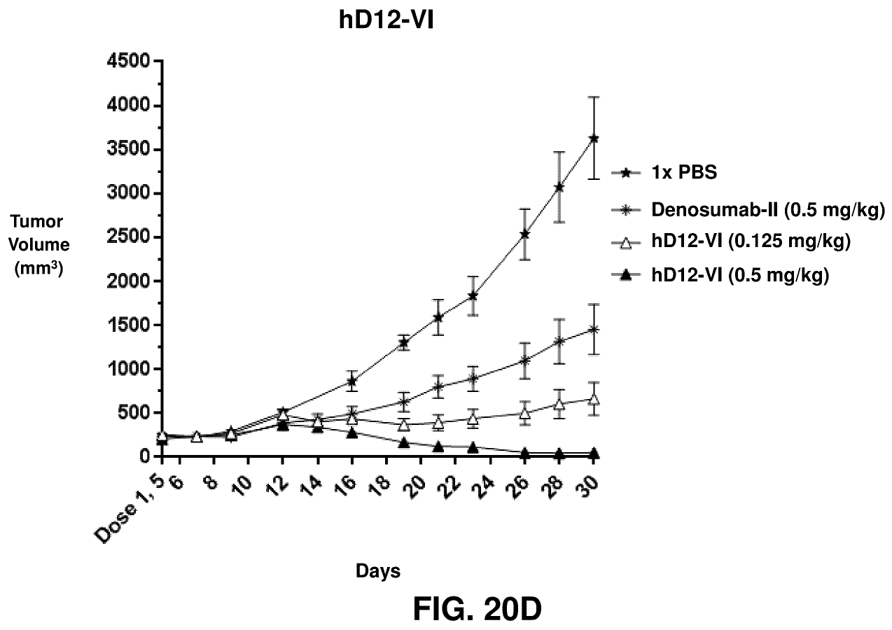

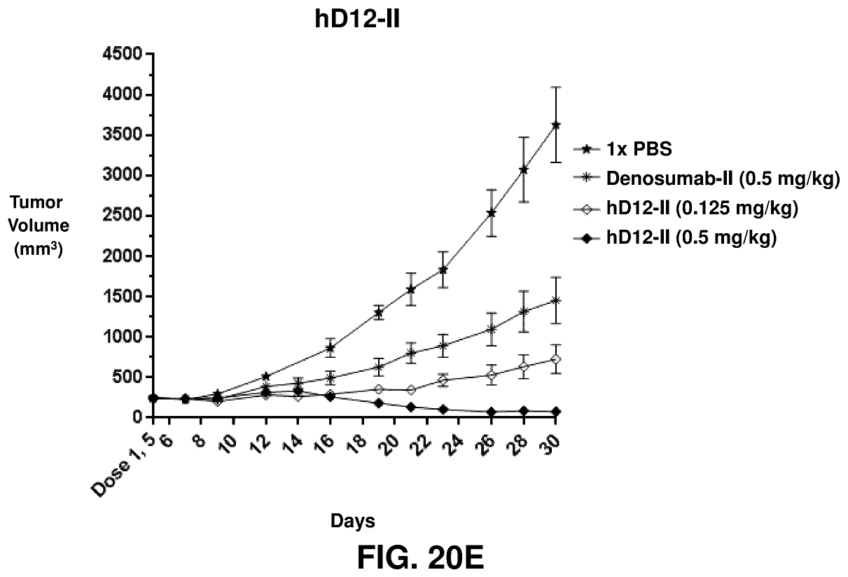

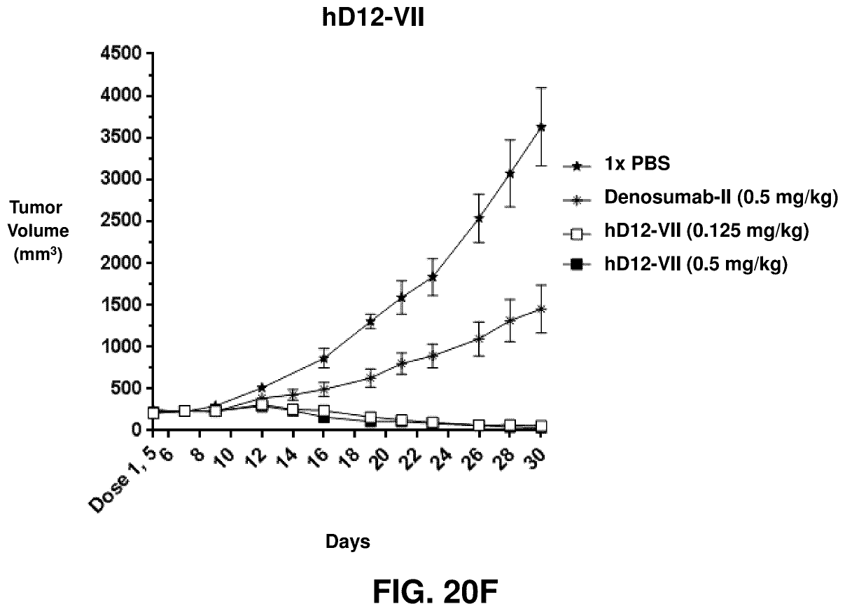

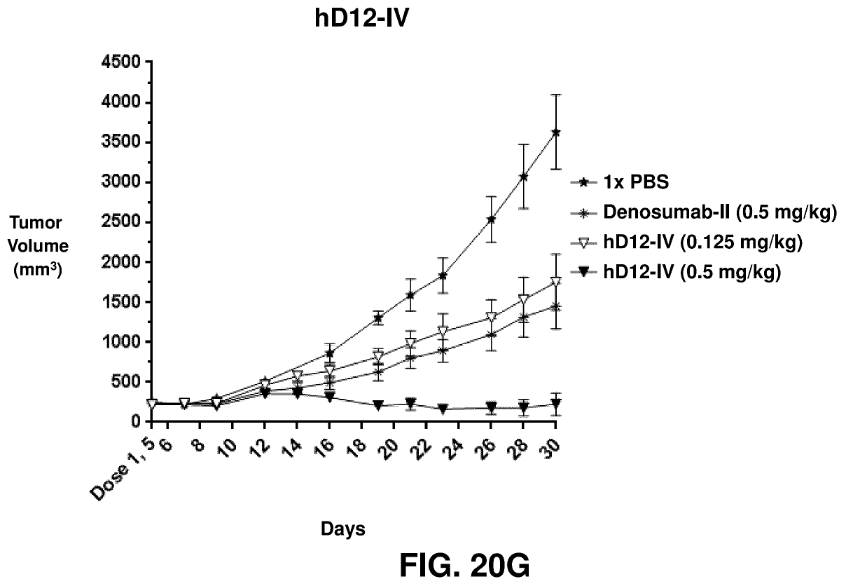

Two in vivo xenograft studies were conducted to assess the efficacy of the five hD12 antibody drug conjugates of Example 3 (i.e., hD12-VI, hD12-II, hD12-VII and hD12-XI). Note that the terms hD12-vc-XI, hD12-vc-VI, hD12-vc-II, hD12-vc-VII and hD12-vc-IV as shown in FIGS. 20-22 are used synonymously with the terms hD12-XI, hD12-VI, hD12-II, hD12-VII and hD12-IV, respectively. Also, Denosumab-* (e.g., Denosumab-II) is used synonymously with the terms Denosumab-vc-* (e.g., Denosumab-vc-II) as shown in the figures. The “vc” designation does not confer any significant meaning. The term “Denosumab-II” refers to the monoclonal antibody “Denosumab” attached to the payload of chemical formula II.

The first H1975 in vivo xenograft study was set up with ten mice (Nu/nu: (Charles River)) in each group. Each mouse was inoculated with H1975 cells followed by treatment with one of the indicated antibody drug conjugates, or with PBS. Antibody drug conjugates were administered as a single administration by i.v. tail vein injection on day 1. Two different doses (0.5 mg/kg and 0.125 mg/kg) of each antibody drug conjugate was tested. Denosumab-II was used as a negative control. Tumor volume and weight were measured 3 times a week. Results of the H1975 in vivo xenograft study are shown in FIGS. 20A-20G.

All animals tolerated the antibody drug conjugates well. There was no significant weight loss observed in any of the groups (e.g., see FIG. 20B). hD12-VII showed the highest efficacy at both dose concentrations of all ADCs tested (FIGS. 20A and 20F). hD12-II and hD12-VI were slightly less efficacious in the low dose group (0.125 mg/kg) (FIGS. 20E and 20D) than hD12-VII. hD12-3315 and hD12-XI were least efficacious in the low dose group (FIGS. 20G and 20C). The isotype control Denosumab-II showed some efficacy at the 0.5 mg/kg dose. It is possible that Denosumab showed tumor growth inhibition because H1975 has a RANK-RANKL signaling pathway (Journal of Thoracic Oncol., 2014, 9 (3) 345-54). In summary, all five hD12 drug conjugates showed significant efficacy against the H1975 xenografts. hD12-VII, hD12-II and hD12-VI showed the highest treatment efficacy with VII slightly outperforming the other two.

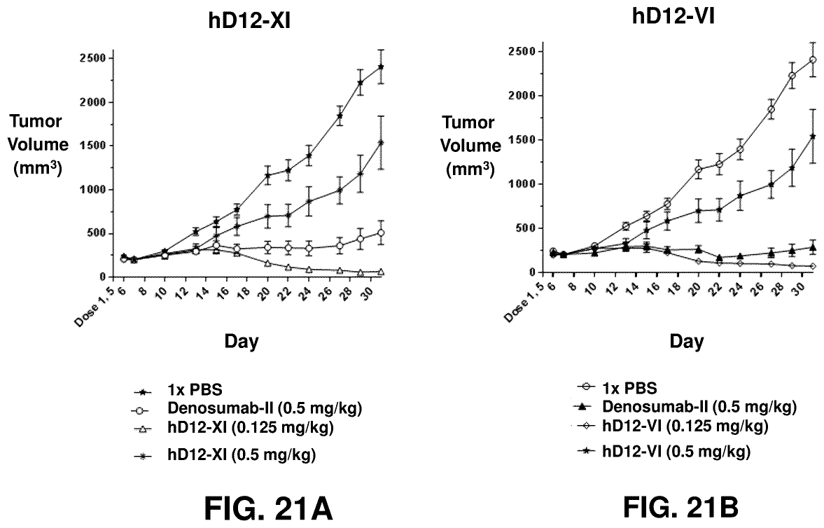

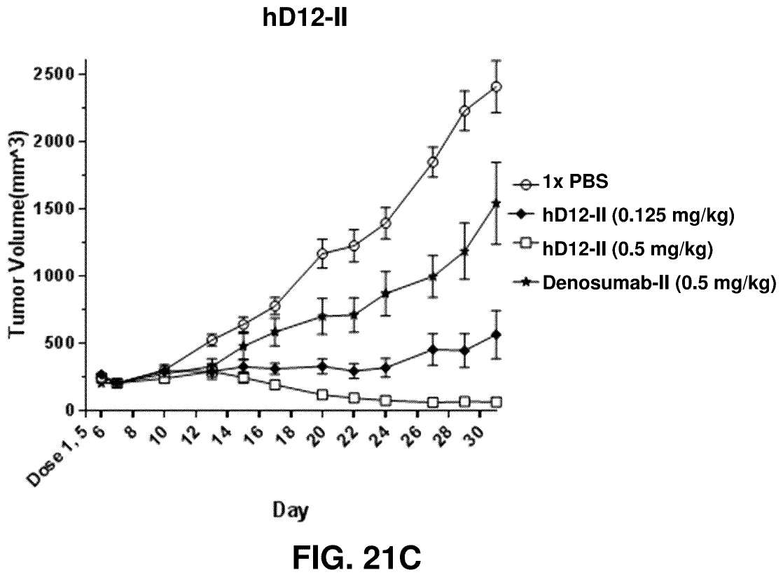

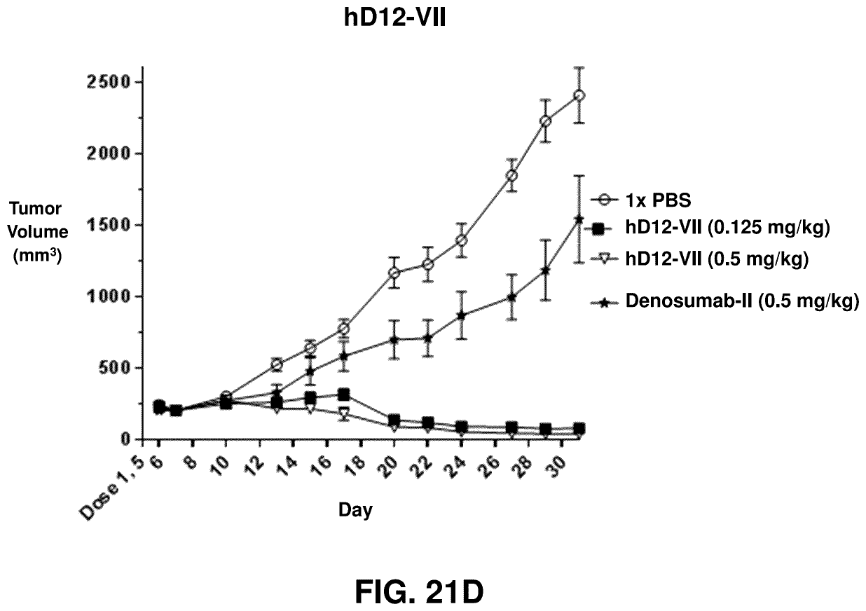

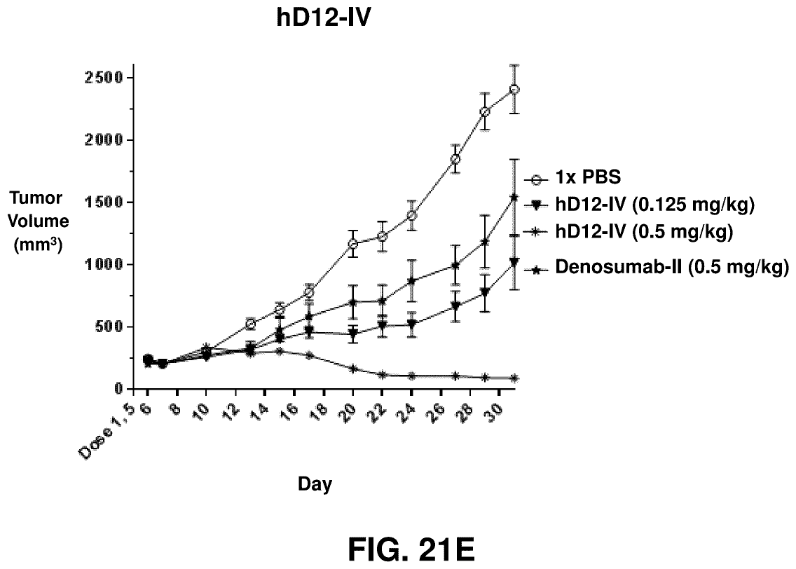

A second H1373 in vivo xenograft study was conducted to further assess the efficacy of the five hD12 antibody drug conjugates of Example 3 (i.e., hD12-VI, hD12-II, hD12-VII and hD12-XI). The H1373 in vivo xenograft study was set up with ten mice (Nu/nu: (Charles River)) in each group. Each mouse was inoculated with H1373 cells followed by treatment with one of the indicated antibody drug conjugates, or with PBS. Antibody drug conjugates were administered as a single administration by i.v. tail vein injection on day 7. Two different doses (0.5 mg/kg and 0.125 mg/kg) of each antibody drug conjugate was tested. Denosumab-II was used as a negative control. Tumor volume and weight were measured 3 times a week. Results of the H1373 in vivo xenograft study are shown in FIGS. 21A-213 and FIG. 22.

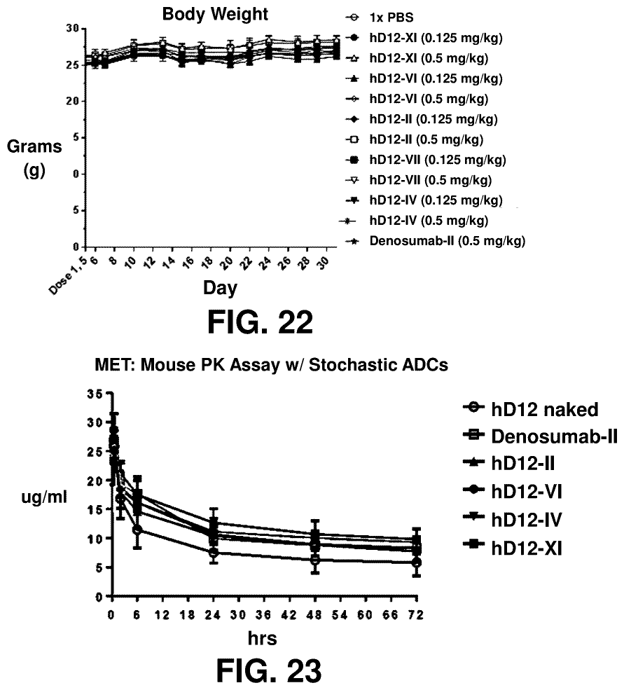

All animals tolerated the antibody drug conjugates well. There was no significant weight loss observed in any of the groups (e.g., see FIG. 22). As seen in the first H1975 xenograft model hD12-VII (FIG. 21D) showed slightly better efficacy in the low dose group (0.125 mg/kg) in comparison to hD12-II and hD12-VI (FIGS. 21C and 21B). hD12-IV was the least efficacious drug, as observed before (FIG. 21E).

### Example 6—PK Studies in Mice

The circulating half-life of the hD12 drug conjugates of Example 3 (i.e., hD12-VI, hD12-II, hD12-VII and hD12-XI) was assessed in 5 groups of 3 mice over a 72 hour period. Each group of mice received a single i.v. injection with 1 mg/kg of one of hD12-II, hD12-IV, hD12-VI, hD12-XI or Denosumab-II. Blood was drawn after 0.5 h, 2 h, 6 h, 24 h, 48 h, 72 h. Serum samples were prepared and analyzed for the amount of each of the indicated antibody drug conjugates (FIG. 23). Serum antibodies were captured with an anti Fc-specific antibody and detected with goat anti-human IgG (H+L)-HRP.

### Example 7—Site-Specific Conjugation of Payloads to hD12

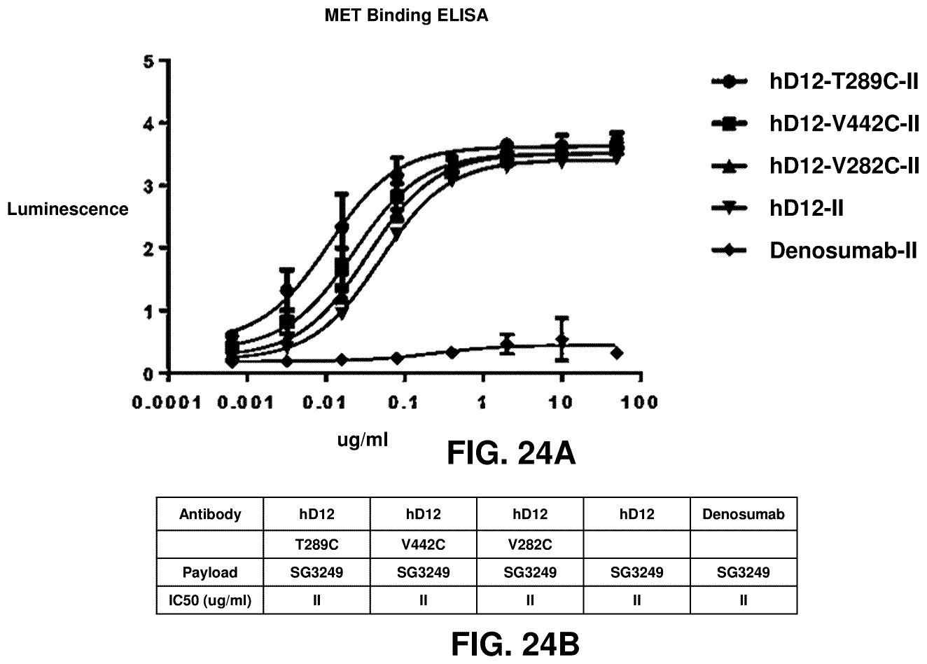

The coding regions of hD12 was mutated at various sites to introduce a cysteine residue into the heavy chain constant region of the IgG2 antibody to obtain the hD12 variant antibodies hD12-T289C (T at position 289 mutated to cysteine), hD12-V442C (V at position 442 mutated to cysteine), hD12-V282C (V at position 282 mutated to cysteine), hD12-S119C (S at position 119 mutated to cysteine). Points of mutation are in the constant region of hD12 and are defined according to the EU numbering system as described in Edelman, G. M. et al. (1969) Proc. Natl. Acad. USA, 63, 78-85. PMID: 5257969. The payload of chemical formula II was site-specifically conjugated to each of the mutated cysteine residues using Maleimide chemistry. The quality and extent of conjugation was assessed by a determination of total recovery, aggregate content, monomer content and drug-antibody ratio (DAR). Optimal conjugation was observed for the hD12 conjugates hD12-T289C-II, hD12-V442C-II and hD12-V282C-II. The relative binding affinity of these three site-specific conjugates for cMET was compared to the stochastically conjugated hD 12-II using a cMET binding ELISA as described in Example 3. The results of the cMET binding assay are shown in FIGS. 24A and 24B. All of the site-specific compounds successfully bound to human cMET with similar affinities. The site specific conjugates bound with similar, or slightly better, affinity than the stochastically coupled hD12-II.

### Example 8—Cytotoxicity Assessment of Site-Specific Conjugates to cMET Expression Cell Lines

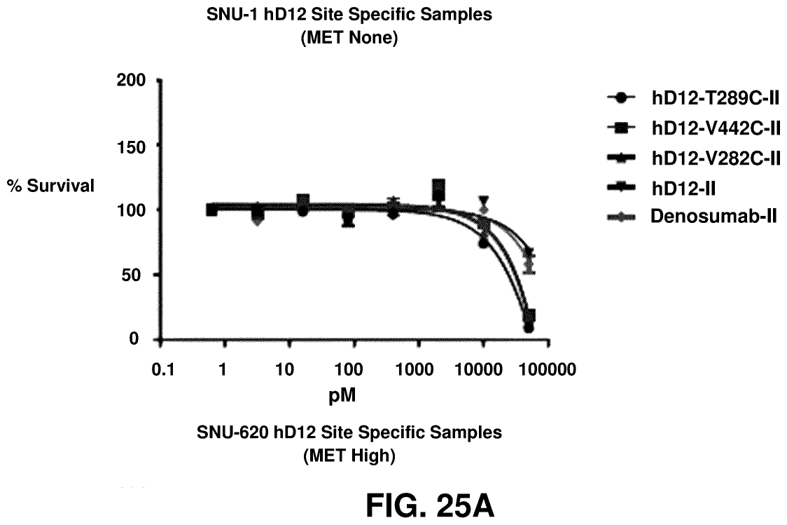

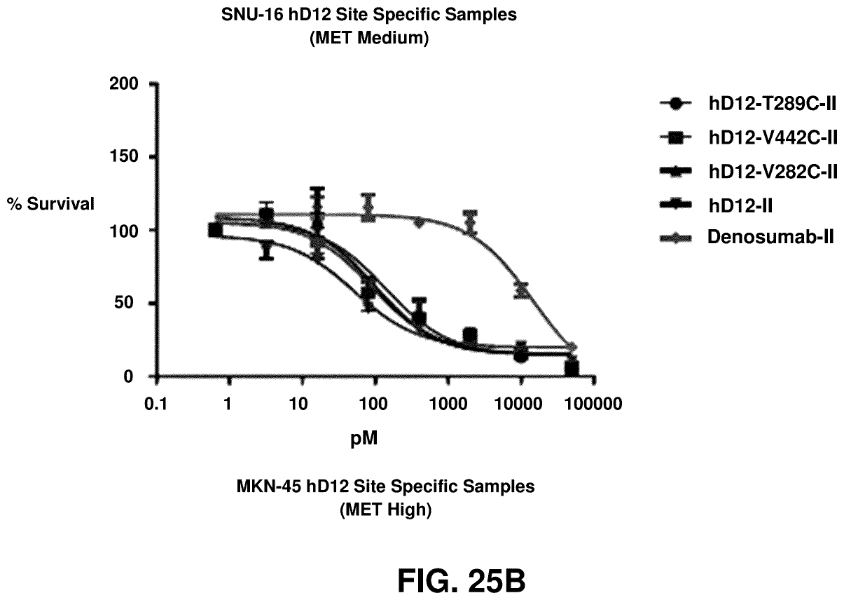

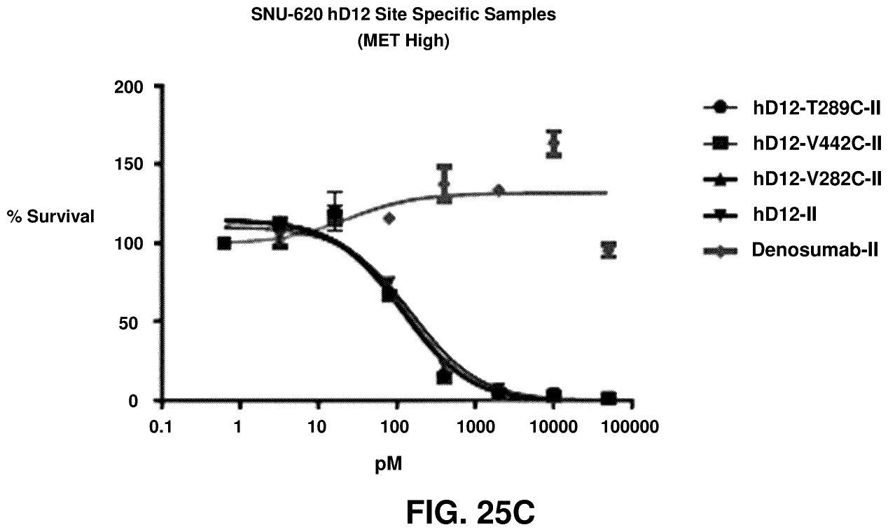

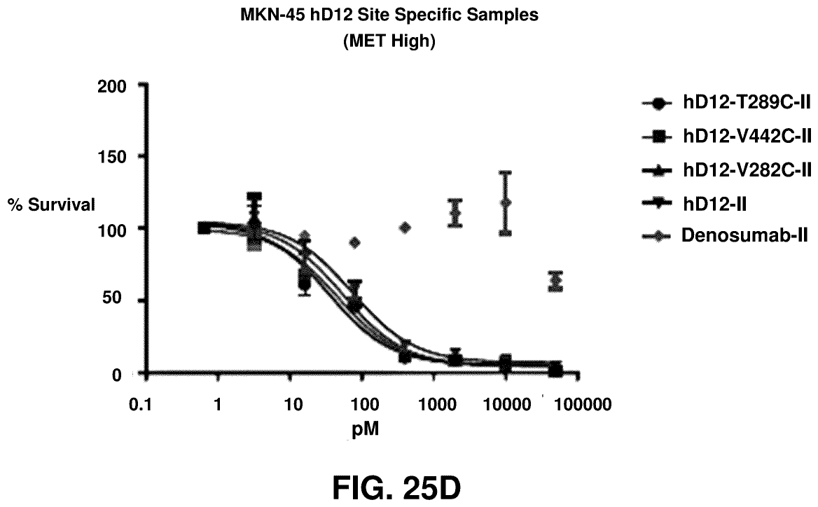

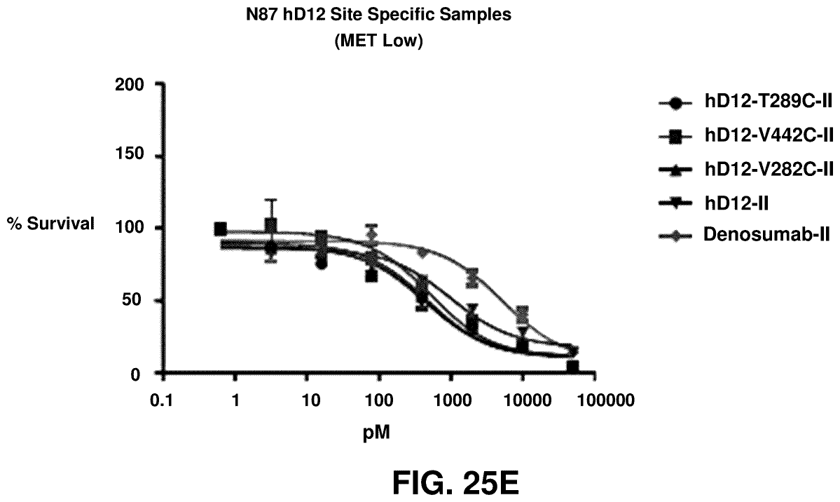

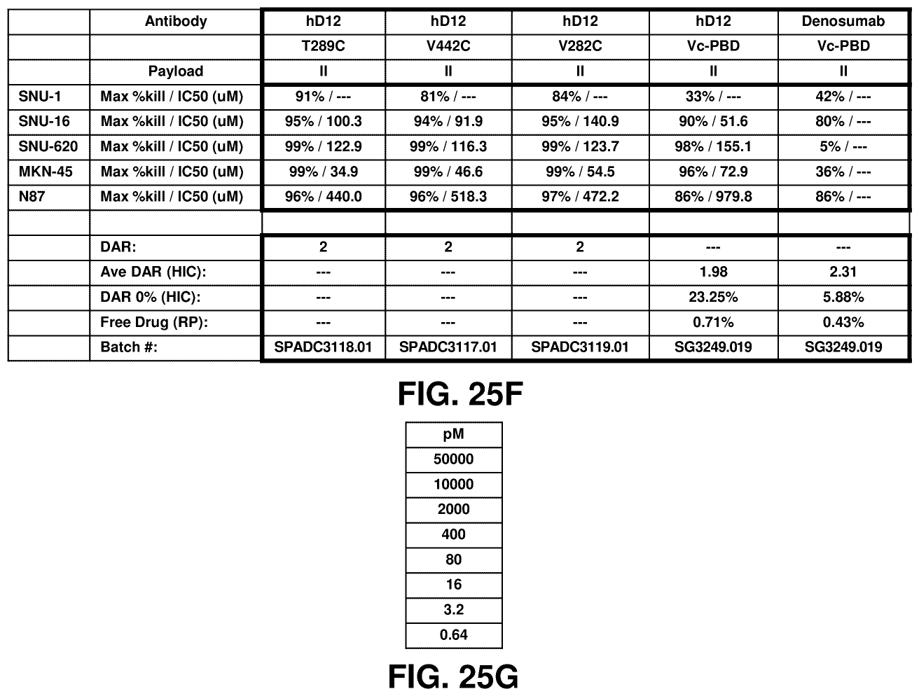

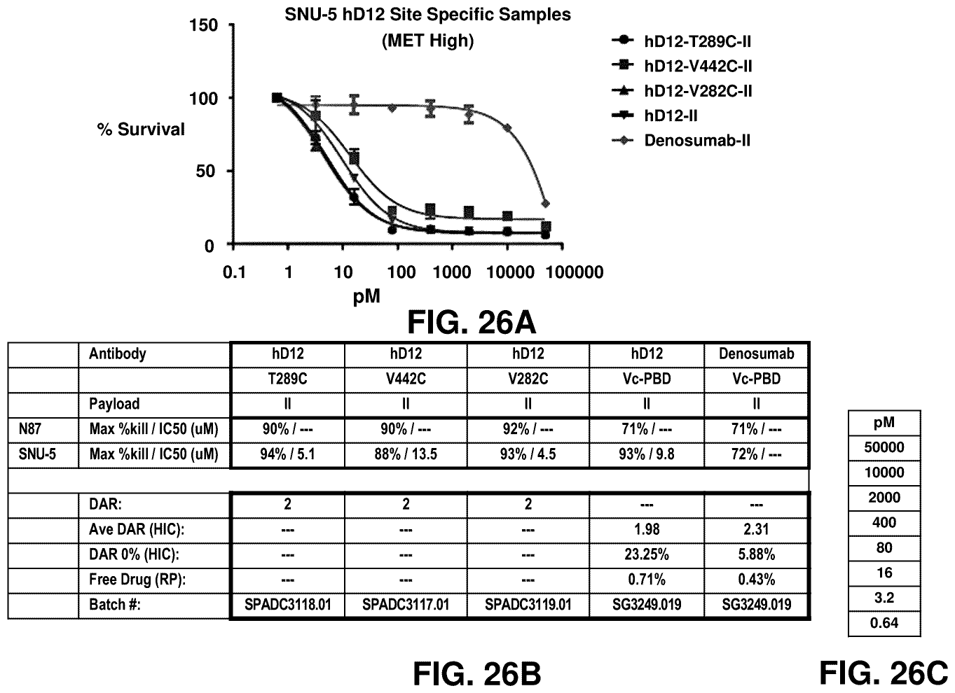

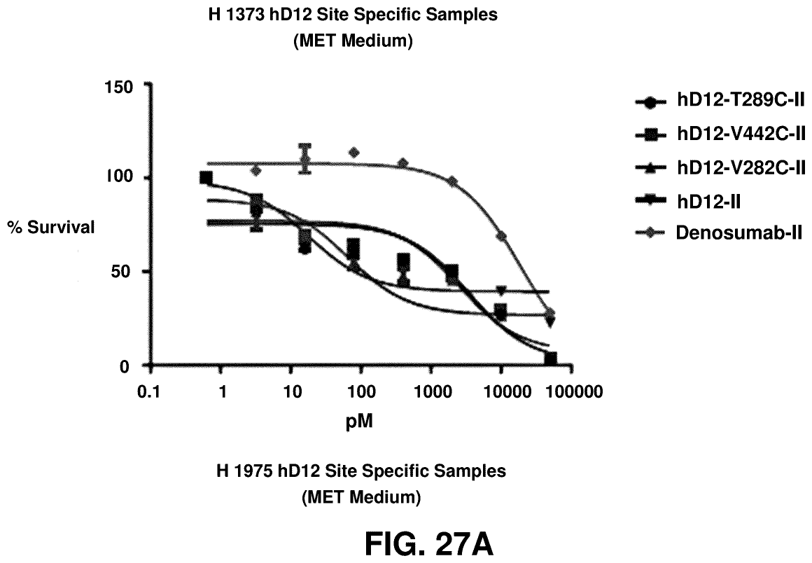

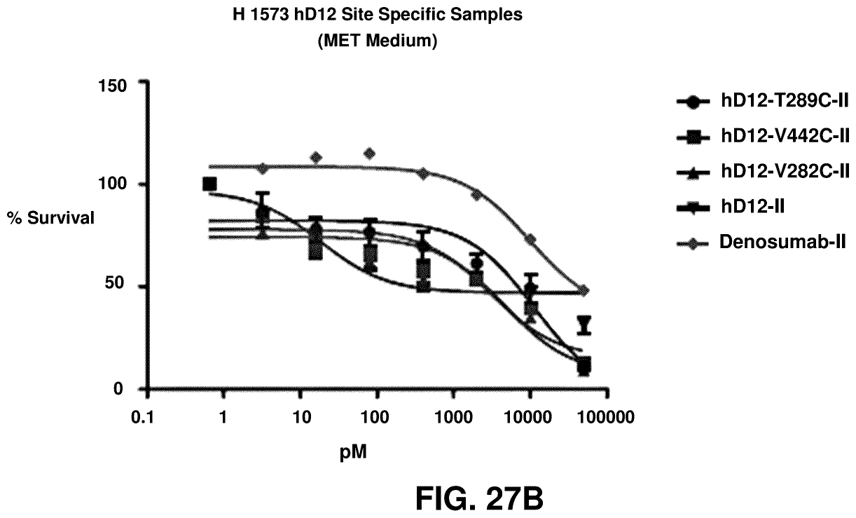

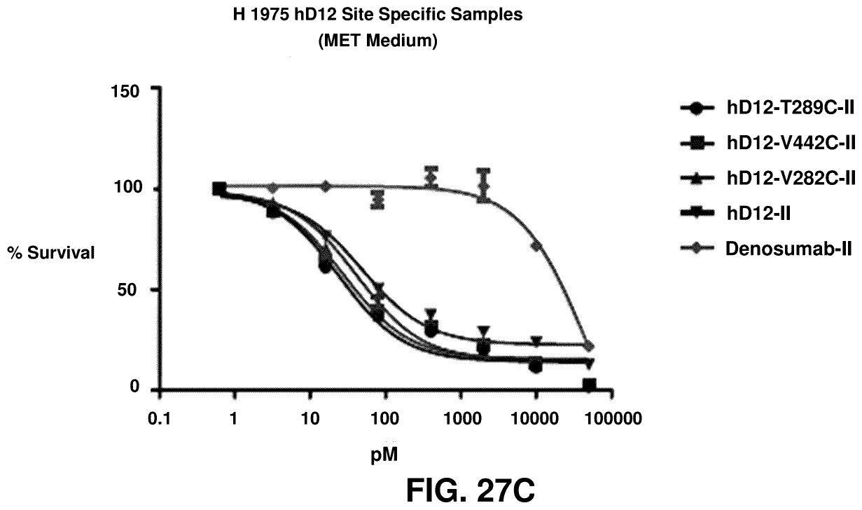

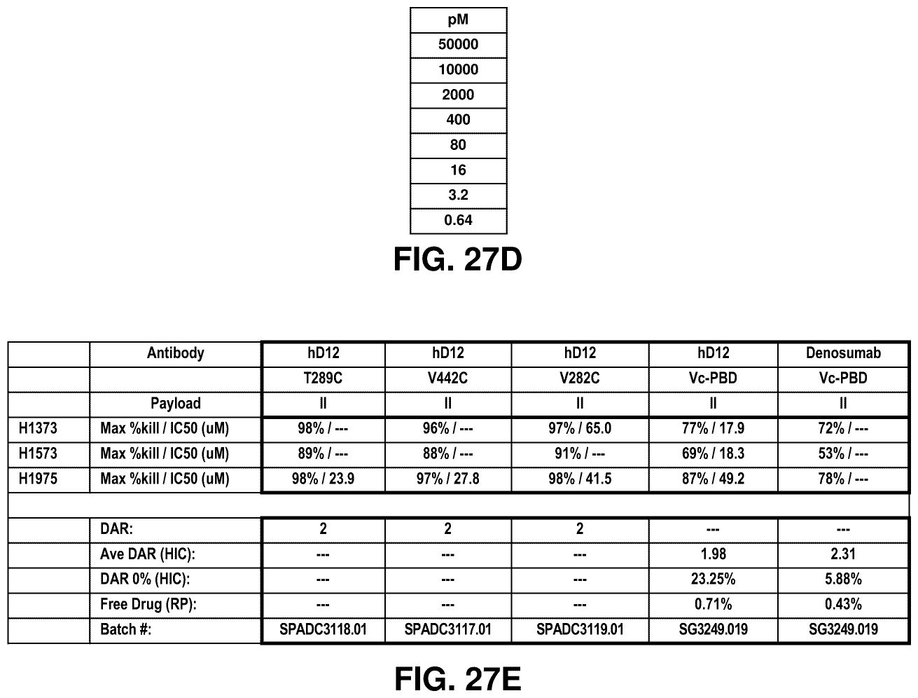

The cytotoxic potency of the site-specifically coupled II conjugates (i.e., hD12-T289C-II, hD12-V442C-II and hD12-V282C-II) was compared to the stochastically coupled hD12-II as a benchmark, against eight cMET expressing cell lines SNU-16 (FIG. 25B), SNU-620 (FIG. 25C), MKN-45 (FIG. 25D), NCI-N87 (FIG. 25E), SNU-5 (FIG. 26A), H1373 (FIG. 27A), H1573 (FIG. 27B), H1975 (FIG. 27C) and the cMET negative cell line SNU-1 (FIG. 25A). Cytotoxicity results are summarized in FIGS. 25F, 26B and 27E. The doses administered are indicated in FIGS. 25G, 26C and 27D.

All site-specific conjugated antibodies showed comparable cytotoxicity to the stochastically coupled hD12-II control against the MKN-45, SNU-620, SNU-5, SNU-16, NCI-N87 and H1975 cell lines. The EC50 for the site-specific conjugates could not be determined for the H1373 and H1573 cell lines due to complex killing curves that differed significantly from sigmoidal curves. The complex shape of the killing curves was more pronounced with the site-specific conjugates, and was repeatable in duplicate experiments. Overall, the cytotoxic potency of the site-specific hD12 conjugates seemed to be better than the stochastically conjugated hD12-II.

### Example 9—In Vivo Xenograft Analysis of Site-Specific Conjugates

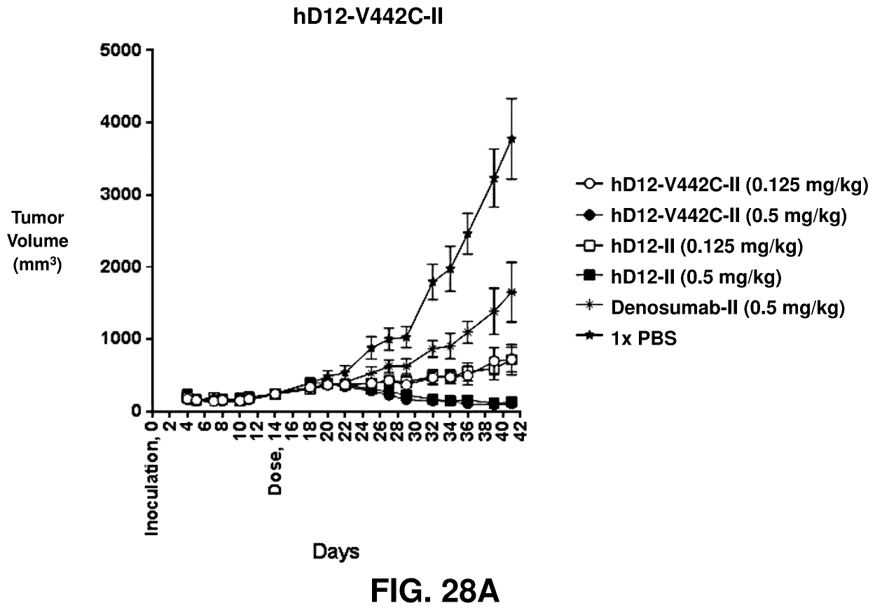

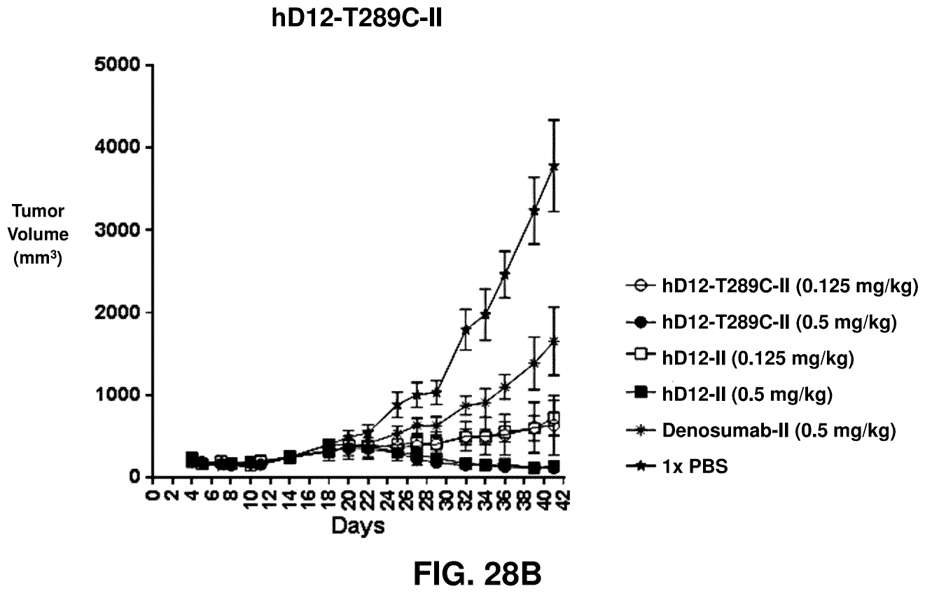

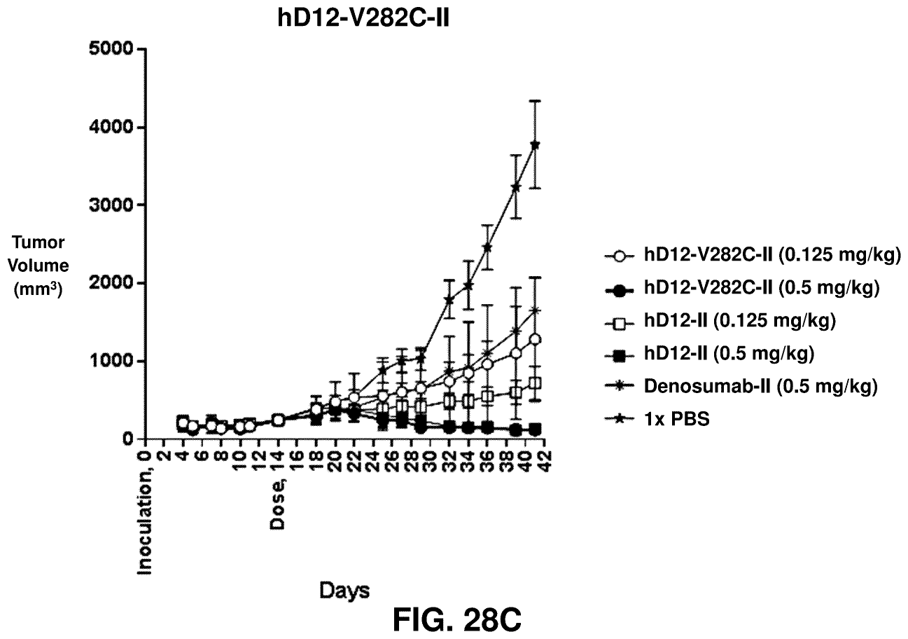

The site-specific conjugates hD12-T289C-II, hD12-V442C-II and hD12-V282C-II and the stochastically coupled hD12-II were assessed in an H1975 (medium cMET expression) xenograft tumor model. Denosumab-II was used as a negative control. Each mouse was inoculated with H1975 cells (Day 0) followed by treatment with one of the indicated antibody drug conjugates, or with PBS. Antibody drug conjugates were administered as a single administration by i.v. tail vein injection on day 14 after tumor cell inoculation. Two different doses (0.5 mg/kg and 0.125 mg/kg) of each antibody drug conjugate were tested. Ten mice were tested for each treatment. Tumor volume and weight were measured 3 times a week. Results of the H1975 in vivo xenograft study are shown in FIGS. 28A-28C.

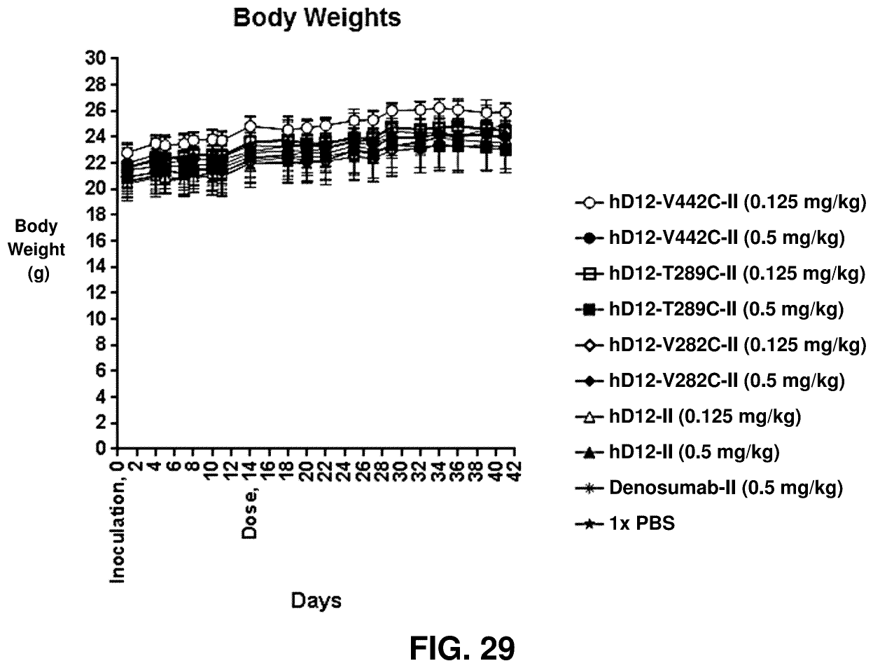

All high dose groups with site-specific ADCs, and the stochastically coupled II (0.5 mg/kg), showed high efficacy in the H1975 model. Tumors regressed completely (FIGS. 28A-28C). The low dose groups (0.125 mg/kg) showed tumor regression with hD12-II (stochastic) and hD12-T289C-II with hD12-V422C-II being the most efficacious. hD12-V282C-II was the least efficacious. The Denosumab control showed mild efficacy in the high dose, which was also seen in the previous H1975 model ran with the stochastically conjugated ADCs. All administered test articles were well tolerated and significant weight loss was not observed (FIG. 29).

### Example 10—Pharmacokinetics in Rats with hD12 Site-Specific Conjugates (Total Antibody Detection)

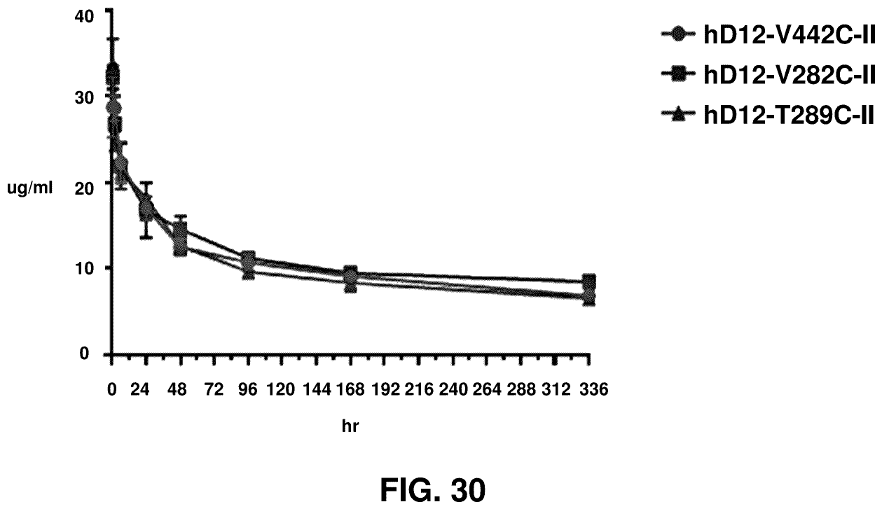

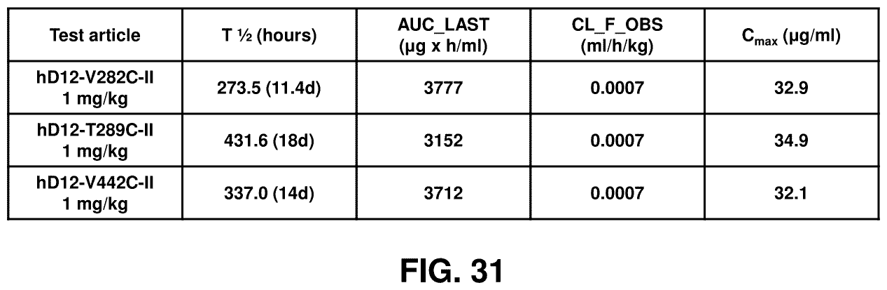

The circulating half-life of the site-specific hD12 conjugates hD12-V422C-II, hD12-V282C-II and hD12-T289C-II was assessed in rats for 21 days. Each group of rats (3 rats/group) received a single i.v. injection with 1 mg/kg of the indicated antibody drug conjugate (FIG. 30). Blood was drawn at 0.5 hr, 2 hr, 6 hr, 24 hr, 48 hr, 72 hr, 168 hr, 312 hr, and 480 hr post injection. Antibody levels in serum samples were analyzed in a PK ELISA using a capture antibody (anti Fc-specific) and goat anti-human IgG (H+L)-HRP detection antibody. The results are summarized in FIG. 31. Pharmacokinetic (PK) parameters of the site-specific conjugates was determined with WinNonlin software. All 3 site-specific conjugates showed similar shaped concentration-time curves. The calculated half-life was between 12 and 18 days. The variants could not be distinguished from one another based on the total antibody pharmacokinetic data alone. Based on the results of this experiment there is no significant pharmacokinetic difference between hD12-V422C-II, hD12-V282C-II and hD12-T289C-II.

### Example 11—Non-Human Primate Tolerability

Stochastically coupled hD12-II and site-specific variants hD12-V282C-II and hD12-T289C-II were tested at amounts up to 1 mg/kg for tolerability in non-human primates. In general all of the antibody drug conjugates were well tolerated. There was no severe body weight loss observed by study end (day 21).

### Example 12—PDX Models, Methods and Results

Patient derived xenografts (PDX) are models of cancer, where tissue or cells from a patient's tumor are implanted into an immunodeficient mouse. PDX models are often used to create an environment that resembles the natural growth of cancer, for the study of cancer progression and treatment. Multiple Crown Bio HuPrime® gastric, colorectal and head and neck (H&N) PDX models were carried out to evaluate the efficacy of hD12-T289C-II. The PDX models were chosen that ranged in their expression of c-Met (low to high).

Briefly, female BALB/c nude mice, 14-15 weeks old, were inoculated subcutaneously in the right flank with a primary human tumor fragment (gastric, colorectal or H&N cancer, 2-3 mm in size) for tumor development. Mice were randomized and grouped (6 treatment groups) when the tumor size reached an average volume of 200 mm³. Each group consisted of 10 mice. Test articles were administered i.v. as a single dose into tumor-bearing mice starting on day 0. If required a second dose was administered. A non-targeting antibody (Secukinumab) linked to the payload of chemical formula II (Secukinumab-II) was administered at a dose of 1 mg/kg. hD12-T289C-II was administered as a single dose of 1.0 mg/kg, 0.5 mg/kg, 0.25 and 0.125 mg/kg, except where noted in FIG. 34. The vehicle control group was administered a single i.v. dose of 1×PBS.

After randomization, tumor size was measured by calipers twice weekly in two dimensions. The tumor volume (mm³) was calculated by TV=0.5a×b² where a and b are the long and short diameters of the tumor surface. Bodyweight was measured and updated along with tumor measurements.

Tumor size was then used to calculate TGI % according to the following formula: TGI %=((mean(C)−mean(C0))−(mean(T)−mean(T0))/(mean(C)−mean(C0))*100% with T-current group value; T0—current group initial value; C—control group value; C0—control group initial value.

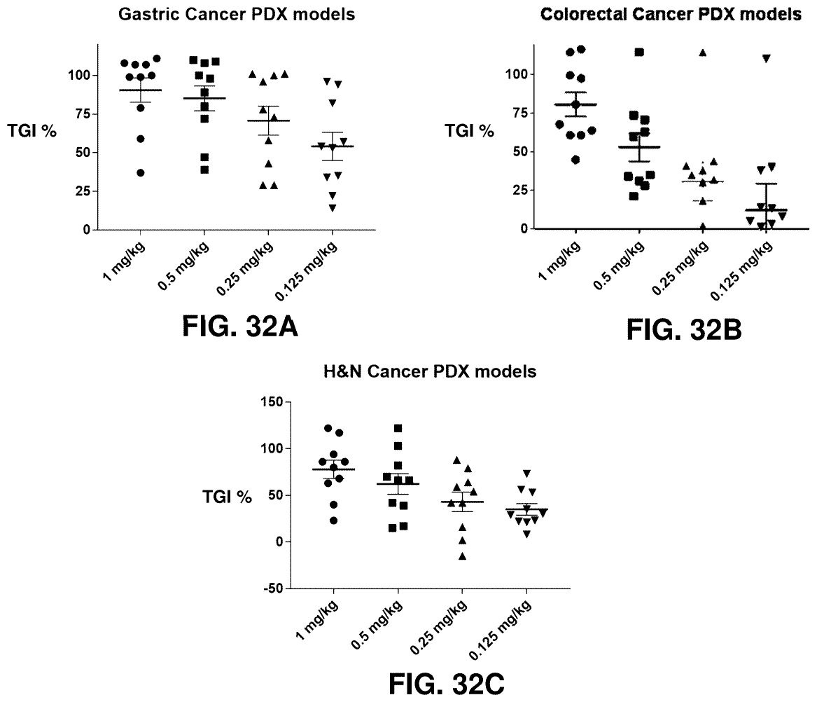

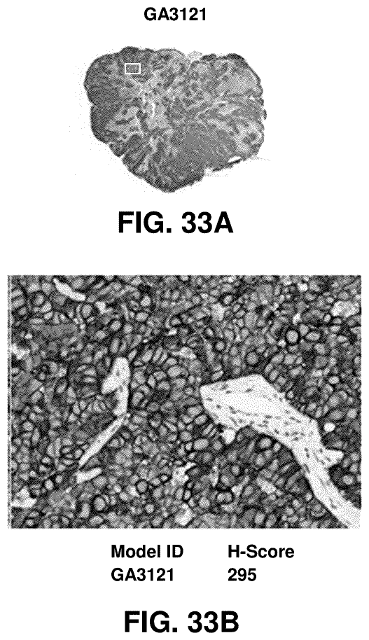

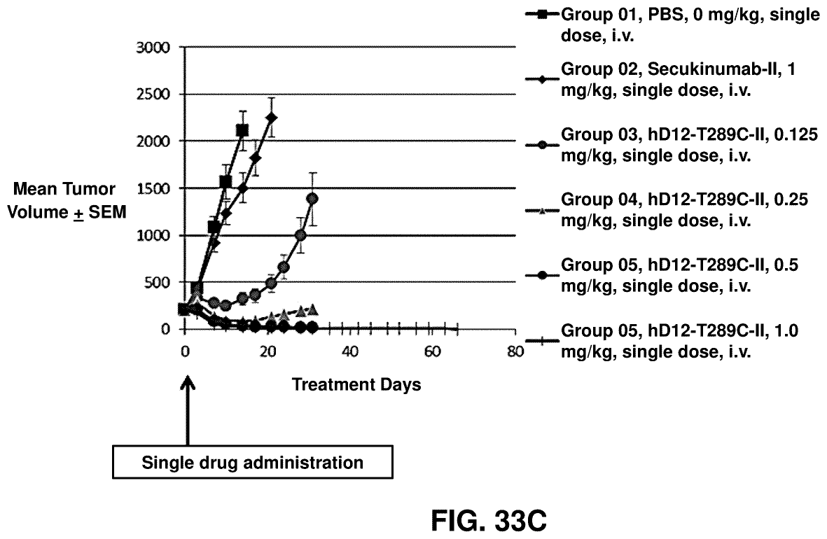

Results of the PDX studies are shown in FIGS. 32 and 33. Each data point represents 1 PDX model group consisting of 10 mice inoculated with a single PDX tumor. TGI % was calculated as described above. Mice were treated with vehicle (PBS) or Secukinumab-II as negative controls. Tumor volume (y-axis) was determined over time (i.e., days, x-axis; FIG. 33C). The results of FIGS. 32 and 33 show that hD12-T289C-II effectively inhibits the growth of human tumor tissues derived from gastric, colorectal or H&N cancer in a dose-dependent manner.

### Example 13

A human subject presents with multiple metastatic carcinomas of 2 cm or larger, present in liver and lung. A biopsy is performed to determine if the cells of the carcinoma express cMET on their cell surface. The presence of cell surface cMET expression is confirmed from the biopsy results.

The human subject is administered a binding agent-drug conjugate described herein that specifically binds to the extracellular domain of human cMET. The binding agent optionally comprises human kappa and IgG2 heavy-chain constant regions, the light chain variable region of SEQ ID NO: 41 and the heavy chain variable region of SEQ ID NO:98. The binding agent-drug conjugate is administered at a dose of 15 mg/kg, intravenously, in a volume of 100 ml over a period of 1 hour, once a day for six weeks. The presence, size and viability of the tumors are determined by follow-up biopsy and ultrasound. The size and number of the tumors are substantially reduced after 2 weeks of treatment. The subject is determined to be in remission after six weeks of treatment.

### Example 14

A human subject presents with colorectal adenocarcinoma having a solid tumor of 2 cm in diameter. The human subject is administered a binding agent-drug conjugate described herein. The binding agent of the binding agent-drug conjugate is a monoclonal humanized antibody of IgG2 isotype comprising a light chain variable region of SEQ ID NO: 47 and a humanized heavy chain variable region of SEQ ID NO: 108. The drug payload has the structure of chemical formula II. The binding agent is administered at a dose of 1 mg/kg, intravenously, in a volume of 50 ml over a period of 30 minutes, once a day for six weeks. After two weeks of treatment, size of the tumor is decreased by more than 50%. The subject is determined to be in remission after six weeks of treatment.

### Example 15—cMET Sequences

(Human cMET-UniProtKB-P08581 (MET_HUMAN)).
*Residue E168 and N375 are bolded and underlined.
SEQ ID NO: 109
MKAPAVLAPGILVLLFTLVQRSNGECKEALAKSEMNVNMKYQLPNFTAETPIQ
NVILHEHHIFLGATNYIYVLNEEDLQKVAEYKTGPVLEHPDCFPCQDCSSKANLSGGVW
KDNINMALVVDTYYDDQLISCGSVNRGTCQRHVFPHNHTADIQSEVHCIFSPQIEEPSQC
PDCVVSALGAKVLSSVKDRFINFFVGNTINSSYFPDHPLHSISVRRLKETKDGFMFLTDQ
SYIDVLPEFRDSYPIKYVHAFESNNFIYFLTVQRETLDAQTFHTRIIRFCSINSGLHSYMEM
PLECILTEKRKKRSTKKEVFNILQAAYVSKPGAQLARQIGASLNDDILFGVFAQSKPDSA
EPMDRSAMCAFPIKYVNDFFNKIVNKNNVRCLQHFYGPNHEHCFNRTLLRNSSGCEAR
RDEYRTEFTTALQRVDLFMGQFSEVLLTSISTFIKGDLTIANLGTSEGRFMQVVVSRSGPS
TPHVNFLLDSHPVSPEVIVEHTLNQNGYTLVITGKKITKIPLNGLGCRHFQSCSQCLSAPP
FVQCGWCHDKCVRSEECLSGTWTQQICLPAIYKVFPNSAPLEGGTRLTICGWDFGFRRN
NKFDLKKTRVLLGNESCTLTLSESTMNTLKCTVGPAMNKHFNMSIIISNGHGTTQYSTFS
YVDPVITSISPKYGPMAGGTLLTLTGNYLNSGNSRHISIGGKTCTLKSVSNSILECYTPAQ
TISTEFAVKLKIDLANRETSIFSYREDPIVYEIHPTKSFISGGSTITGVGKNLNSVSVPRMVI
NVHEAGRNFTVACQHRSNSEIICCTTPSLQQLNLQLPLKTKAFFMLDGILSKYFDLIYVH
NPVFKPFEKPVMISMGNENVLEIKGNDIDPEAVKGEVLKVGNKSCENIHLHSEAVLCTV
PNDLLKLNSELNIEWKQAISSTVLGKVIVQPDQNFTGLIAGVVSISTALLLLLGFFLWLKK
RKQIKDLGSELVRYDARVHTPHLDRLVSARSVSPTTEMVSNESVDYRATFPEDQFPNSS
QNGSCRQVQYPLTDMSPILTSGDSDISSPLLQNTVHIDLSALNPELVQAVQHVVIGPSSLI
VHFNEVIGRGHFGCVYHGTLLDNDGKKIHCAVKSLNRITDIGEVSQFLTEGIIMKDFSHP
NVLSLLGICLRSEGSPLVVLPYMKHGDLRNFIRNETHNPTVKDLIGFGLQVAKGMKYLA
SKKFVHRDLAARNCMLDEKFTVKVADFGLARDMYDKEYYSVHNKTGAKLPVKWMAL
ESLQTQKFTTKSDVWSFGVLLWELMTRGAPPYPDVNTFDITVYLLQGRRLLQPEYCPDP
LYEVMLKCWHPKAEMRPSFSELVSRISAIFSTFIGEHYVHVNATYVNVKCVAPYPSLLSS
EDNADDEVDTRPASFWETS
(Rat cMET-UniProtKB-P97523 (MET_RAT))
SEQ ID NO: 110
MKAPTALAPGILLLLLTLAQRSHGECKEALVKSEMNVNMKYQLPNFTAETPIH
NVVLPGHHIYLGATNYIYVLNDKDLQKVSEFKTGPVVEHPDCFPCQDCSSKANVSGGV
WKDNVNMALLVDTYYDDQLISCGSVNRGTCQRHVLPPDNAADIQSEVHCMFSPLAEEE
SGQCPDCVVSALGAKVLLSEKDRFINFFVGNTINSSYPPDYSLHSISVRRLKETQDGFKFL
TDQSYIDVLPEFRDSYPIKYIHAFESNHFIYFLTVQKETLDAQTFHTRIIRFCSVDSGLHSY
MEMPLECILTEKRRKRSTREEVFNILQAAYVSKPGANLAKQIGASPYDDILYGVFAQSKP
DSAEPMNRSAVCAFPIKYVNDFFNKIVNKNNVRCLQHFYGPNHEHCFNRTLLRNSSGCE
VRSDEYRTEFTTALQRVDLFMGRLNHVLLTSISTFIKGDLTIANLGTSEGRFMQVVLSRT
AHFTPHVNFLLDSYPVSPEVIVEHPSNQNGYTLVVTGKKITKIPLNGLGCGHFQSCSQCL
SPPYFIQCGWCHNRCVHSNECPSGTWTQEICLPAVYKVFPTSAPLEGGTMLTICGWDFG
FKKNNKFDLRKTKVLLGNESCTLTLSESTTNTLKCTVGPAMSEHFNVSVIVSNSRETTQY
SAFSYVDPVITSISPRYGPHAGGTLLTLTGKYLNSGNSRHISIGGKTCTLKSVSDSILECYT
PGHTVSAEFPVKLKIDLADRVTSSFSYREDPVVSEIHPTKSFISGGSTITGIGKNLNSVSTP
KLVIEVHDVGVNYTVACQHRSSSEIICCTTPSLRQLDLQLPLKTKAFFLLDGILSKHFDLT
YVHDPMFKPFEKPVMISMGNENVVEIKGDDIDPEAVKGEVLKVGNKSCENLHWHSEAL
LCTVPSDLLKLNGGELNIEWKQAVSSTVLGKVIVQPDQNFAGLIIGAVSISVVVLLVSGL
FLWLRKRKHKDLGSELVRYDARVHTPHLDRLVSARSVSPTTEMVSNESVDYRATFPED
QFPNSSQNGACRQVQYLLTDLSPILTSGDSDISSPLLQNTVHIDLSALNPELVQAVPHVVI
GPSSLIVHFNEVIGRGHFGCVYHGTLLDSDGKKIHCAVKSLNRITDIEEVSQFLTEGIIMK
DFSHPNVLSLLGICLRSEGSPLVVLPYMKHGDLRNFIRNETHNPTVKDLIGFGLQVAKG
MKYLVSKKFVHRDLAARNCMLDEKFTVKVADFGLARDMYDKEYYSVHNKTGAKLPV
KWMALESLQTQKFTTKSDVWSFGVLLWELMTRGAPPYPDVNTFDITIYLLQGRRLLQPE
YCPDALYEVMLKCWHPKAEMRPS VSELVSRISSIFSTFIGEHYVHVNATYVNVKCVAPY
PSLLPSQDNIDGEANT
(Mouse cMET-UniProtKB-P16056 (MET_MOUSE))
SEQ ID NO: 111
MKAPTVLAPGILVLLLSLVQRSHGECKEALVKSEMNVNMKYQLPNFTAETPIQ
NVVLHGHHIYLGATNYIYVLNDKDLQKVSEFKTGPVLEHPDCLPCRDCSSKANSSGGV
WKDNINMALLVDTYYDDQLISCGSVNRGTCQRHVLPPDNSADIQSEVHCMFSPEEESGQ
CPDCVVSALGAKVLLSEKDRFINFFVGNTINSSYPPGYSLHSISVRRLKETQDGFKFLTDQ
SYIDVLPEFLDSYPIKYIHAFESNHFIYFLTVQKETLDAQTFHTRIIRFCSVDSGLHSYMEM
PLECILTEKRRKRSTREEVFNILQAAYVSKPGANLAKQIGASPSDDILFGVFAQSKPDSAE
PVNRSAVCAFPIKYVNDFFNKIVNKNNVRCLQHFYGPNHEHCFNRTLLRNSSGCEARSD
EYRTEFTTALQRVDLFMGRLNQVLLTSISTFIKGDLTIANLGTSEGRFMQVVLSRTAHLT
PHVNFLLDSHPVSPEVIVEHPSNQNGYTLVVTGKKITKIPLNGLGCGHFQSCSQCLSAPY
FIQCGWCHNQCVRFDECPSGTWTQEICLPAVYKVFPTSAPLEGGTVLTICGWDFGFRKN
NKFDLRKTKVLLGNESCTLTLSESTTNTLKCTVGPAMSEHFNVSVIISNSRETTQYSAFSY
VDPVITSISPRYGPQAGGTLLTLTGKYLNSGNSRHISIGGKTCTLKSVSDSILECYTPAQTT
SDEFPVKLKIDLANRETSSFSYREDPVVYEIHPTKSFISGGSTITGIGKTLNSVSLPKLVIDV
HEVGVNYTVACQHRSNSEIICCTTPSLKQLGLQLPLKTKAFFLLDGILSKHFDLTYVHNP
VFEPFEKPVMISMGNENVVEIKGNNIDPEAVKGEVLKVGNQSCESLHWHSGAVLCTVPS
DLLKLNSELNIEWKQAVSSTVLGKVIVQPDQNFAGLIIGAVSISVVVLLLSGLFLWMRKR
KHKDLGSELVRYDARVHTPHLDRLVSARSVSPTTEMVSNESVDYRATFPEDQFPNSSQN
GACRQVQYPLTDLSPILTSGDSDISSPLLQNTVHIDLSALNPELVQAVQHVVIGPSSLIVHF
NEVIGRGHFGCVYHGTLLDNDGKKIHCAVKSLNRITDIEEVSQFLTEGIIMKDFSHPNVLS
LLGICLRSEGSPLVVLPYMKHGDLRNFIRNETHNPTVKDLIGFGLQVAKGMKYLASKKF
VHRDLAARNCMLDEKFTVKVADFGLARDMYDKEYYSVHNKTGAKLPVKWMALESLQ
TQKFTTKSDVWSFGVLLWELMTRGAPPYPDVNTFDITIYLLQGRRLLQPEYCPDALYEV
MLKCWHPKAEMRPSFSELVSRISSIFSTFIGEHYVHVNATYVNVKCVAPYPSLLPSQDNI
DGEGNT
(Dog cMET)
SEQ ID NO: 112
MKAPAVLAPGILVLLFTLVQKSYGECKEALVKSEMNVNMKYQLPNFTAETPIQ
NVVLHKHHIYLGAVNYIYVLNDKDLQKVAEYKTGPVLEHPDCSPCQDCSHKANLSGGV
WEDNINMALLVDTYYDDQLISCGSVHRGTCQRHILPPSNIADIQSEVHCMYSSQADEEPS
QCPDCVVSALGTKVLISEKDRFINFFVGNTINSSDHPDHSLHSISVRRLKETQDGFKFLTD
QSYIDVLPEFRDSYPIKYVHAFESNHFIYFLTVQRETLDAQTFHTRIIRFCSVDSGLHSYM
EMPLECILTEKRRKRSTREEVFNILQAAYVSKPGAHLAKQIGANLNDDILYGVFAQSKPD
SAEPMNRSAVCAFPIKYVNEFFNKIVNKNNVRCLQHFYGPNHEHCFNRTLLRNSSGCEA
RNDEYRTEFTTALQRVDLFMGQFNQVLLTSISTFIKGDLTIANLGTSEGRFMQVVVSRSG
LSTPHVNFRLDSHPVSPEAIVEHPLNQNGYTLVVTGKKITRIPLNGLGCEHFQSCSQCLSA
PPFVQCGWCHDRCVHLEECPTGAWTQEVCLPAIYEVFPTSAPLEGGTVLTVCGWDFGF
RRNNKFDLKKTKVFLGNESCTLTLSESTTNMLKCTVGPAVNEHFNISIIISNGRGTAQYST
FSYVDPIITSISPSYGPKNGGTLLTLTGKYLNSGNSRHISMGGKTCTLKSVSDSILECYTPA
QATATEFPIKLKIDLANREMNSFSYQEDPIVYAIHPTKSFISGGSTITAVGKNLNSVSVLR
MVIDVHETRRNFTVACQHRSNSEIICCTTPSLQQLNLQLPLKTKAFFMLDGIHSKYFDLIY
VHNPVFKPFEKPVMISIGNENVLEIKGNDIDPEAVKGEVLKVGNKSCETIYSDSKAVLCK
VPNDLLKLNNELNIEWKQAVSSTVLGKVIVQPDQNFTGLIAGVISISTIVLLLLGLFLWLK
RKKQIKDLGSELVRYDARVHTPHLDRLVSARSVSPTTEMVSNESVDYRATFPEDQFPNS
SQNGSCRQVQYPLTDLSPMLTSGDSDISSPLLQNTVHIDLSALNPELVQAVQHVVIGPSS
LIVHFNEVIGRGHFGCVYHGTLLDNDDKKIHCAVKSLNRITDIGEVSQFLTEGIIMKDFSH
PNVLSLLGICLRSEGSPLVVLPYMKHGDLRNFIRNETHNPTVKDLIGFGLQVAKGMKYL
ASKKFVHRDLAARNCMLDEKFTVKVADFGLARDMYDKEYYSVHNKTGAKLPVKWM
ALESLQTQKFTTKSDVWSFGVLLWELMTRGAPPYPDVNTFDITVYLLQGRRLLQPEYCP
DPLYEVMLKCWHPRAELRPSFSELVSRISAIFSTFIGEHYVHVNATYVNVKCVAPYPSLL
SSQDNIDGEGDT
(Macaca mulatta, Rhesus cMET-NCBI Reference Sequence:
NP_001162100.1)
SEQ ID NO: 113
MKAPAVLVPGILVLLFTLVQRSNGECKEALAKS EMNVNMKYQLPNFTAETAIQ
NVILHEHHIFLGATNYIYVLNEEDLQKVAEYKTGPVLEHPDCFPCQDCSSKANLSGGVW
KDNINMALVVDTYYDDQLISCGSVNRGTCQRHVFPHNHTADIQSEVHCIFSPQIEEPNQC
PDCVVSALGAKVLSSVKDRFINFFVGNTINSSYFPHHPLHSISVRRLKETKDGFMFLTDQ
SYIDVLPEFRDSYPIKYIHAFESNNFIYFLTVQRETLNAQTFHTRIIRFCSLNSGLHSYMEM
PLECILTEKRKKRSTKKEVFNILQAAYVSKPGAQLARQIGASLNDDILFGVFAQSKPDSA
EPMDRSAMCAFPIKYVNDFFNKIVNKNNVRCLQHFYGPNHEHCFNRTLLRNSSGCEAR
RDEYRAEFTTALQRVDLFMGQFSEVLLTSISTFVKGDLTIANLGTSEGRFMQVVVSRSGP
STPHVNFLLDSHPVSPEVIVEHPLNQNGYTLVVTGKKITKIPLNGLGCRHFQSCSQCLSAP
PFVQCGWCHDKCVRSEECPSGTWTQQICLPAIYKVFPTSAPLEGGTRLTICGWDFGFRR
NNKFDLKKTRVLLGNESCTLTLSESTMNTLKCTVGPAMNKHFNMSIIISNGHGTTQYSTF
SYVDPIITSISPKYGPMAGGTLLTLTGNYLNSGNSRHISIGGKTCTLKSVSNSILECYTPAQ
TISTEFAVKLKIDLANRETSIFSYREDPIVYEIHPTKSFISGGSTITGVGKNLHSVSVPRMVI
NVHEAGRNFTVACQHRSNSEIICCTTPSLQQLNLQLPLKTKAFFMLDGILSKYFDLIYVH
NPVFKPFEKPVMISMGNENVLEIKGNDIDPEAVKGEVLKVGNKSCENIHLHSEAVLCTV
PNDLLKLNSELNIEWKQAISSTVLGKVIVQPDQNFTGLIAGVVSISIALLLLLGLFLWLKK
RKQIKDLGSELVRYDARVHTPHLDRLVSARSVSPTTEMVSNESVDYRATFPEDQFPNSS
QNGSCRQVQYPLTDMSPILTSGDSDISSPLLQNTVHIDLSALNPELVQAVQHVVIGPSSLI
VHFNEVIGRGHFGCVYHGTLLDNDGKKIHCAVKSLNRITDIGEVSQFLTEGIIMKDFSHP
NVLSLLGICLRSEGSPLVVLPYMKHGDLRNFIRNETHNPTVKDLIGFGLQVAKGMKYLA
SKKFVHRDLAARNCMLDEKFTVKVADFGLARDMYDKEYYSVHNKTGAKLPVKWMAL
ESLQTQKFTTKSDVWSFGVLLWELMTRGAPPYPDVNTFDITVYLLQGRRLLQPEYCPDP
LYEVMLKCWHPKAEMRPSFSELVSRISAIFSTFIGEHYVHVNATYVNVKCVAPYPSLLSS
EDNADDEVDT
Heavy Chain sequence of hD12
SEQ ID NO: 114
QVQLVQSGAEVKKPGASVKVSCKASGYTFTSYWMHWVKQAPGQGLDWIGYI
KPSTDNTEYNQKFKDKATLTADKSTSTAYMELSSLRSEDTAVYYCARSYGNYPLMDY
WGQGTTVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECP
PCPAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGK
Light Chain sequence of hD12
SEQ ID: 115
QIVLTQSPAILSLSPGERATLSCSASSSVTSNYLYWYQQKPGSSPKLLIYSTSNLA
SGVPARFSGSGSGTSYTLTISSLEAEDAASYFCHQWSSYPPTFGSGTKLEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Heavy Chain sequence of hD12 T289C (Residue C289
is underlined)
SEQ ID NO: 116
QVQLVQSGAEVKKPGASVKVSCKASGYTFTSYWMHWVKQAPGQGLDWIGYI
KPSTDNTEYNQKFKDKATLTADKSTSTAYMELSSLRSEDTAVYYCARSYGNYPLMDY
WGQGTTVTVSSASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDKTVERKCCVECP
PCPAPPVAGPSVFLFPPKPKDTLMISRTPEVTCVVVDVSHEDPEVQFNWYVDGVEVHNA
KCKPREEQFNSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGK

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology

1. (canceled)

2. The binding agent-drug conjugate of claim 79, wherein the pyrrolobenzodiazepine toxin comprises the structure of chemical formula I: 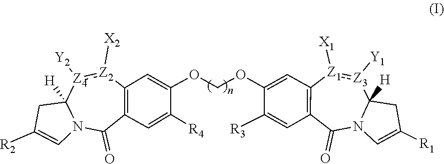
wherein
Z₁ and Z₂ are both N;
Z₃ and Z₄ are both C;
the double-dash lines [symbol] represent a single bond or a double bond;
n is 1 to 10;
each of R₃ and R₄ are independently H, or a C₁₋₄ alkoxyl; and
each of R₁ and R₂ are independently selected from the group consisting of H, C₁₋₅ alkyl, C₃₋₆ cycloalkyl, C₂₋₅ alkenyl, and a phenyl optionally substituted with R₅, wherein
R₅ is selected from the group consisting of —NH₂, —NHR₆, and a piperazinyl substituted with R₇ having the structure 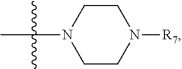
wherein R₆ comprises the linking group, and
R₇ is H, or a C₁₋₅ alkyl;
X₁ is null, a protecting group, or comprises the linking group;
X₂ is null, a protecting group, or comprises the linking group;
only one of X₁, X₂, R₁, and R₂ comprises the linking group; and
each of Y₁ and Y₂ are independently either null, OH, or SO₃H;
provided that:
(i) when X₁ comprises the linking group, Z₁ [symbol] Z₃ is N—C,
(ii) when X₂ comprises the linking group, Z₂ [symbol] Z₄ is N—C,
(iii) when X₁ comprises the protecting group, Z₁ [symbol] Z₃ is N—C, and
(iv) when X₂ comprises the protecting group, Z₂ [symbol] Z₄ is N—C,
wherein null indicates the absence of the moiety or the presence of one or more hydrogens to complete a required valency.

3-14. (canceled)

15. The binding agent-drug conjugate of claim 79, wherein the linking group comprises the structure of chemical formula A: 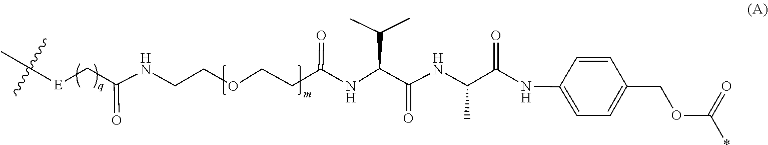
wherein
the asterisk indicates the point of attachment to the pyrrolobenzodiazepine toxin;
the wavy line indicates the point of attachment to the binding agent;
m is 1 to 20;
q is 0 to 10; and
E is a connecting group.

16-18. (canceled)

19. The binding agent-drug conjugate of claim 79, wherein the linking group comprises the structure of chemical formula B: 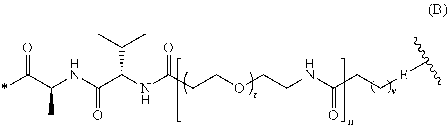
wherein
the asterisk indicates the point of attachment to the pyrrolobenzodiazepine toxin;
the wavy line indicates the point of attachment to the binding agent;
E is a connecting group;
v is 0 to 10; and
u is 0 or 1; wherein when u is 1, t is 1 to 10.

20-24. (canceled)

25. The binding agent-drug conjugate of claim 79, wherein the protecting group has the following structure (D): 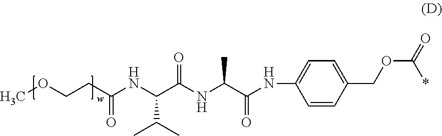
wherein the asterisk indicates the point of attachment to the pyrrolobenzodiazepine toxin; and w is 1 to 5.

26-27. (canceled)

28. The binding agent-drug conjugate of claim 79, wherein the payload comprises a structure selected from the group consisting of 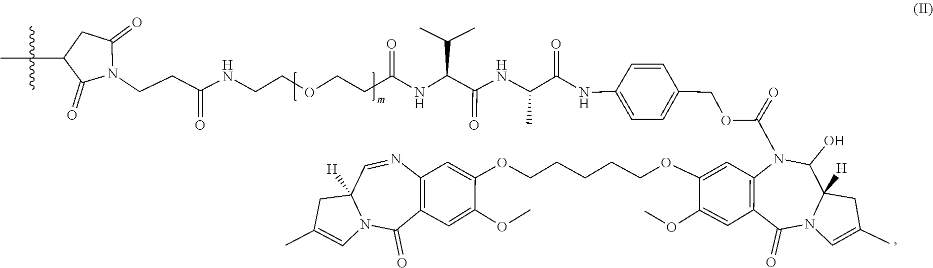
wherein m is 8; 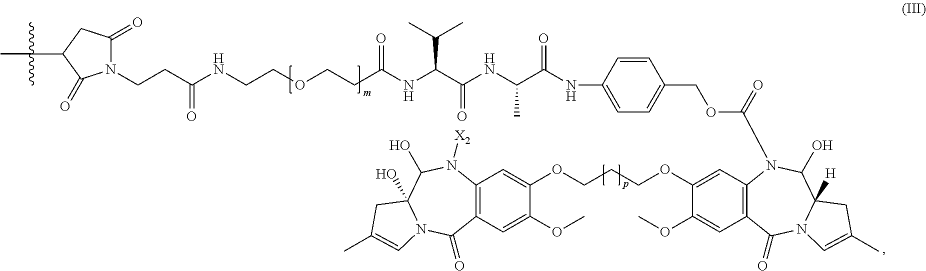
wherein m is 8, p is 3, and X₂ is a protecting group; 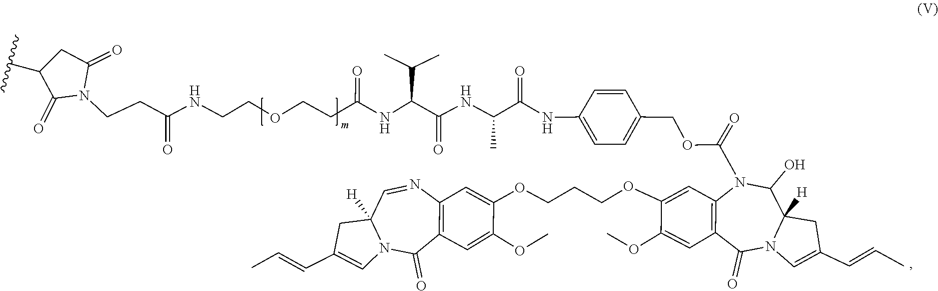
wherein m is 8; 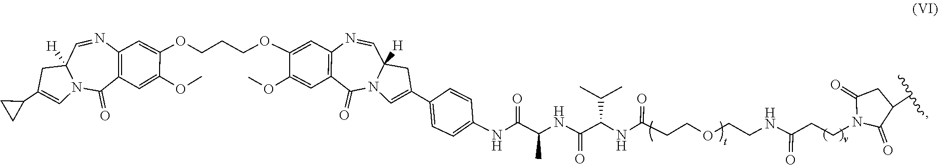
wherein t is 8, and v is 1; and 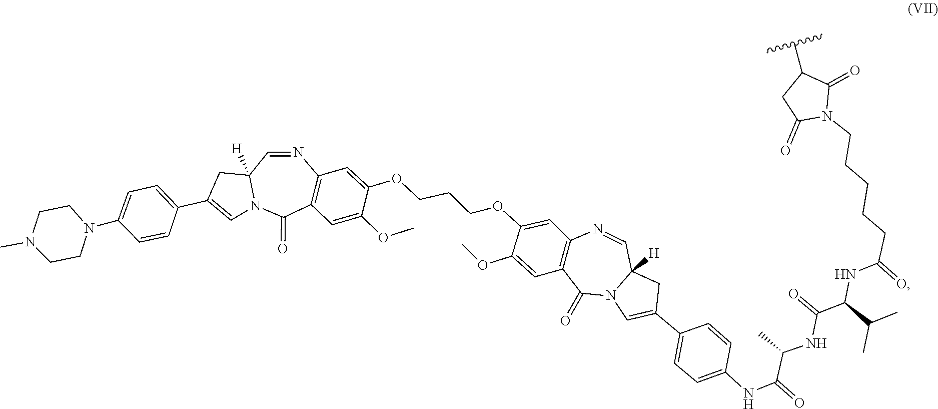 [SMILES: *C1CC(=O)N(CCCCCC(=O)N[C@H](C(=O)N[C@@H](C)C(=O)Nc2ccc(C3=CN4C(=O)c5cc(OC)c(OCCCOc6cc7c(cc6OC)C(=O)N6C=C(c8ccc(N9CCN(C)CC9)cc8)C[C@H]6C=N7)cc5N=C[C@@H]4C3)cc2)C(C)C)C1=O]
wherein the wavy line indicates the point of attachment to the binding agent.

29. The binding agent-drug conjugate of claim 28, wherein the protecting group of X₂ has the following structure (D): 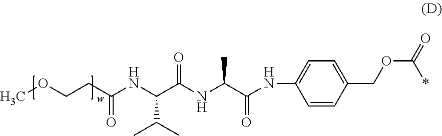
wherein the asterisk indicates the point of attachment to the payload; and
w is 1 to 5.

30-35. (canceled)

36. The binding agent-drug conjugate of claim 79, wherein the binding agent is an antibody, or a binding fragment thereof.

37-55. (canceled)

56. The binding agent-drug conjugate of claim 79, wherein the binding agent binds specifically to a mammalian cMET.

57-60. (canceled)

61. The binding agent-drug conjugate of claim 79, wherein the binding agent specifically binds to the extracellular domain of a cMET variant.

62-67. (canceled)

68. The binding agent-drug conjugate of claim 79, wherein the binding agent comprises a light chain variable region comprising SEQ ID NO: 47 and a heavy chain variable region comprising SEQ ID NO: 108.

69-72. (canceled)

73. A method of treating a subject having a neoplastic disorder or cancer comprising:
a) providing a subject having, or suspected of having, a neoplastic disorder or cancer; and
b) administering to the subject a therapeutically effective amount of the binding agent-drug conjugate of claim 79.

74-75. (canceled)

76. The method of claim 73, wherein the neoplastic disorder or cancer comprises a carcinoma, sarcoma, neuroblastoma, glioblastoma, myeloma, lymphoma, melanoma or a solid or soft tissue tumor.

77. The method of claim 76, wherein the neoplastic disorder or cancer comprises a bladder cancer, breast cancer, colorectal cancer, gastric cancer, pancreatic cancer, esophageal cancer, liver cancer, hepatocellular cancer, hypopharynx cancer, lung cancer, adenocarcinoma, ovarian cancer or renal cancer.

78. The method of claim 76, wherein the neoplastic disorder or cancer comprises a pancreatic adenocarcinoma, pancreatic neuroendocrine cancer, colorectal adenocarcinoma, small intestinal malignancy, cholangiocarcinoma, non-small cell lung cancer (NSCLC), thyroid carcinoma, esophageal or esophagogastric junction (EGJ) cancer, gastric adenocarcinoma, liver hepatocellular carcinoma, head and neck squamous carcinoma, female genital tract malignancy, breast carcinoma, triple negative breast cancer, lung small cell carcinoma, ovarian surface epithelial carcinoma, retroperitoneal or peritoneal sarcoma, prostatic adenocarcinoma, neuroendocrine tumor, gastrointestinal stromal tumor, glioblastoma or non-epithelial ovarian cancer.

79. A binding agent-drug conjugate comprising a binding agent and a payload, wherein the binding agent comprises: a heavy chain and light chain each having CDRs that are the same as respective CDRs of heavy chain and light chain of an antibody produced by hybridoma cell line F6B1P3D12, deposited with ATCC on Mar. 20, 2019 under ATCC PTA-125826 and
the payload comprises a pyrrolobenzodiazepine toxin and a linking group;
wherein the pyrrolobenzodiazepine toxin is covalently linked to the linking group, the linking group is covalently linked to the binding agent, and the binding agent specifically binds to an extracellular domain of mesenchymal epithelial factor (cMET).


SEQUENCE LISTING (116 sequences)

SEQ ID NO: 1 - protein, 16 aa, Mus musculus
RSSQTIVHGTGNTYLE

SEQ ID NO: 2 - protein, 11 aa, Mus musculus
QTIVHGTGNTY

SEQ ID NO: 3 - protein, 11 aa, Mus musculus
KASENVGTYVS

SEQ ID NO: 4 - protein, 6 aa, Mus musculus
ENVGTY

SEQ ID NO: 5 - protein, 17 aa, Mus musculus
RSSQSLLYSINQKNYLA

SEQ ID NO: 6 - protein, 12 aa, Mus musculus
QSLLYSINQKNY

SEQ ID NO: 7 - protein, 11 aa, Mus musculus
RASENIYNTLA

SEQ ID NO: 8 - protein, 6 aa, Mus musculus
ENIYNT

SEQ ID NO: 9 - protein, 12 aa, Mus musculus
SASSSVTSNYLY

SEQ ID NO: 10 - protein, 7 aa, Mus musculus
SSVTSNY

SEQ ID NO: 11 - protein, 12 aa, Mus musculus
SASSSVSSNYLY

SEQ ID NO: 12 - protein, 7 aa, Mus musculus
SSVSSNY

SEQ ID NO: 13 - protein, 12 aa, Mus musculus
  VAR_SEQ 7: note = misc_feature - X is S or T
SASSSVXSNYLY

SEQ ID NO: 14 - protein, 7 aa, Mus musculus
QSVTSNY

SEQ ID NO: 15 - protein, 12 aa, Mus musculus
RASQSVTSNYLY

SEQ ID NO: 16 - protein, 7 aa, Mus musculus
KVSNRFS

SEQ ID NO: 17 - skipped

SEQ ID NO: 18 - protein, 7 aa, Mus musculus
GASNRYT

SEQ ID NO: 19 - skipped

SEQ ID NO: 20 - protein, 7 aa, Mus musculus
WASTRES

SEQ ID NO: 21 - skipped

SEQ ID NO: 22 - protein, 7 aa, Mus musculus
AATNLAD

SEQ ID NO: 23 - skipped

SEQ ID NO: 24 - protein, 7 aa, Mus musculus
STSNLAS

SEQ ID NO: 25 - skipped

SEQ ID NO: 26 - protein, 20 aa, Mus musculus
FQGSHVPYTFGGGTKLEIKR

SEQ ID NO: 27 - protein, 9 aa, Mus musculus
FQGSHVPYT

SEQ ID NO: 28 - protein, 20 aa, Mus musculus
GQSYSYPLTFGAGTKLELKR

SEQ ID NO: 29 - protein, 9 aa, Mus musculus
GQSYSYPLT

SEQ ID NO: 30 - protein, 19 aa, Mus musculus
QQYYTYPLTFGAGTKLELK

SEQ ID NO: 31 - protein, 9 aa, Mus musculus
QQYYTYPLT

SEQ ID NO: 32 - protein, 19 aa, Mus musculus
QHFWGTPYTFGGGTKLEIK

SEQ ID NO: 33 - protein, 9 aa, Mus musculus
QHFWGTPYT

SEQ ID NO: 34 - protein, 19 aa, Mus musculus
HQWSSYPPTFGSGTKLEIK

SEQ ID NO: 35 - protein, 9 aa, Mus musculus
HQWSSYPPT

SEQ ID NO: 36 - protein, 9 aa, Mus musculus
  VAR_SEQ 1: note = misc_feature - Xaa is His, Gln or Gly
  VAR_SEQ 3: note = misc_feature - Xaa is Trp, Ser or Tyr
  VAR_SEQ 4: note = misc_feature - Xaa is Ser or Tyr
  VAR_SEQ 5: note = misc_feature - Xaa is Ser or Thr
  VAR_SEQ 8: note = misc_feature - Xaa is Pro or Leu
XQXXXYPXT

SEQ ID NO: 37 - protein, 113 aa, Mus musculus
DVLMTQTPLSLPVSLGDQASISCRSSQTIVHGTGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 38 - protein, 108 aa, Mus musculus
DIVMTQSPKSMSMSVGERVTLSCKASENVGTYVSWYQQKPDQSPKLLIYGASNRYTGVPDRFTGSGSATDFTLTISSVQAEDLADYHCGQSYSYPLTFGAGTKLELKR

SEQ ID NO: 39 - protein, 114 aa, Mus musculus
DIVMSQSPSSLAVSVGEKVTMSCRSSQSLLYSINQKNYLAWYQQIPGQSPKLLIYWASTRESGVPDRFTGSGSGTDFTLTISRVKAEDLALYYCQQYYTYPLTFGAGTKLELKR

SEQ ID NO: 40 - protein, 110 aa, Mus musculus
RCDIQMTQSPASLSVSVGETVTITCRASENIYNTLAWYLQKQGKSPQLLVYAATNLADGVPSRFSGSGSGTQYSLKINSLQSEDFGSYYCQHFWGTPYTFGGGTKLEIKR

SEQ ID NO: 41 - protein, 109 aa, Mus musculus
QIVLTQSPAIMSASPGEKVTLTCSASSSVTSNYLYWYQQKPGSSPKLWIYSTSNLASGVPARFSGSGSGTSYSLTISSMEAEDAASYFCHQWSSYPPTFGSGTKLEIKR

SEQ ID NO: 42 - protein, 109 aa, Mus musculus
QIVLTQSPAIMSASPGEKVTLTCSASSSVSSNYLYWYQQKPGSSPKLWIYSTSNLASGVPVRFSGSGSGTSYSLTISSMEAEDAASYFCHQWSSYPPTFGSGTKLEIKR

SEQ ID NO: 43 - protein, 109 aa, Mus musculus
QIVLTQSPAIMSASPGEKVTLTCSASSSVSSNYLYWYHQKPGSSPKLWIYSTSNLASGVPVRFSGSGSGTSYSLTISSMEAEDAASYFCHQWSSYPPTFGSGTKLEIKR

SEQ ID NO: 44 - protein, 108 aa, Mus musculus
  VAR_SEQ 38: note = MISC_FEATURE - Xaa is His or Gln
  VAR_SEQ 61: note = MISC_FEATURE - Xaa is Ala or Arg
QIVLTQSPAIMSASPGEKVTLTCSASSSVSSNYLYWYXQKPGSSPKLWIYSTSNLASGVPXFSGSGSGTSYSLTISSMEAEDAASYFCHQWSSYPPTFGSGTKLEIKR

SEQ ID NO: 45 - protein, 109 aa, synthetic construct; identical to SEQ ID NO: 41
  REGION 1..109: note = Humanized Light Chain

SEQ ID NO: 46 - protein, 109 aa, synthetic construct
  REGION 1..109: note = Humanized Light Chain
QIVLTQSPATMSASPGERVTLSCSASSSVTSNYLYWYQQKPGSSPRLWIYSTSNLASGVPARFSGSGSGTSYTLTISRMEPEDAASYFCHQWSSYPPTFGSGTKLEIKR

SEQ ID NO: 47 - protein, 109 aa, synthetic construct
  REGION 1..109: note = Humanized Light Chain
QIVLTQSPAILSLSPGERATLSCSASSSVTSNYLYWYQQKPGSSPKLLIYSTSNLASGVPARFSGSGSGTSYTLTISSLEAEDAASYFCHQWSSYPPTFGSGTKLEIKR

SEQ ID NO: 48 - protein, 109 aa, synthetic construct
  REGION 1..109: note = Humanized Light Chain
QIVLTQSPATLSLSPGERATLSCRASQSVTSNYLYWYQQKPGSSPRLLIYSTSNLASGVPARFSGSGSGTDYTLTISRLEPEDFASYFCHQWSSYPPTFGSGTKLEIKR

SEQ ID NO: 49 - protein, 109 aa, synthetic construct
  REGION 1..109: note = Humanized Light Chain
QIVLTQSPATLSLSPGERATLSCSASSSVTSNYLYWYQQKPGSSPRLLIYSTSNLASGVPARFSGSGSGTSYTLTISRLEPEDFASYFCHQWSSYPPTFGSGTKLEIKR

SEQ ID NO: 50 - protein, 10 aa, Mus musculus
GFSLTNYGVN

SEQ ID NO: 51 - protein, 8 aa, Mus musculus
GFSLTNYG

SEQ ID NO: 52 - protein, 10 aa, Mus musculus
GFNINDYFMH

SEQ ID NO: 53 - protein, 7 aa, Mus musculus
FNINDYF

SEQ ID NO: 54 - protein, 10 aa, Mus musculus
GFTFTDYYMS

SEQ ID NO: 55 - protein, 8 aa, Mus musculus
GFTFTDYY

SEQ ID NO: 56 - protein, 10 aa, Mus musculus
GYTFTDYNMD

SEQ ID NO: 57 - protein, 7 aa, Mus musculus
YTFTDYN

SEQ ID NO: 58 - protein, 10 aa, Mus musculus
GYTFTSYWMH

SEQ ID NO: 59 - protein, 7 aa, Mus musculus
YTFTSYW

SEQ ID NO: 60 - protein, 8 aa, Mus musculus
  VAR_SEQ 6: note = MISC_FEATURE - Xaa is Asp or Ser
  VAR_SEQ 8: note = MISC_FEATURE - Xaa is Asn or Trp
GYTFTXYX

SEQ ID NO: 61 - protein, 10 aa, Mus musculus
  VAR_SEQ 2: note = misc_feature - Xaa is Tyr or Phe
  VAR_SEQ 6: note = misc_feature - Xaa is Asp or Ser
  VAR_SEQ 8: note = misc_feature - Xaa is Asn, Trp or Tyr
  VAR_SEQ 10: note = misc_feature - Xaa is His or Ser
GXTFTXYXMX

SEQ ID NO: 62 - protein, 16 aa, Mus musculus
LIWGGGDTDYNSALKS

SEQ ID NO: 63 - protein, 7 aa, Mus musculus
IWGGGDT

SEQ ID NO: 64 - protein, 17 aa, Mus musculus
WIDPENGNTIYDPKFQG

SEQ ID NO: 65 - protein, 8 aa, Mus musculus
IDPENGNT

SEQ ID NO: 66 - protein, 19 aa, Mus musculus
FIRNKANGYTTKYSASVKG

SEQ ID NO: 67 - protein, 10 aa, Mus musculus
IRNKANGYTT

SEQ ID NO: 68 - protein, 17 aa, Mus musculus
DINPNNGGTIYNQKFKG

SEQ ID NO: 69 - protein, 8 aa, Mus musculus
INPNNGGT

SEQ ID NO: 70 - protein, 17 aa, Mus musculus
YIKPSTDNTEYNQKFKD

SEQ ID NO: 71 - protein, 8 aa, Mus musculus
IKPSTDNT

SEQ ID NO: 72 - protein, 17 aa, Mus musculus
YINPSTDYTEYNQKFKD

SEQ ID NO: 73 - protein, 8 aa, Mus musculus
INPSTDYT

SEQ ID NO: 74 - protein, 17 aa, Mus musculus
YINPSTDYIEYNQKFKD

SEQ ID NO: 75 - protein, 8 aa, Mus musculus
INPSTDYI

SEQ ID NO: 76 - protein, 8 aa, Mus musculus
  VAR_SEQ 8: note = misc_feature - Xaa is Ile or Thr
INPSTDYX

SEQ ID NO: 77 - protein, 17 aa, Mus musculus
  VAR_SEQ 1: note = misc_feature - Xaa is Tyr or Asp
  VAR_SEQ 3: note = misc_feature - Xaa is Lys or Asn
  VAR_SEQ 8: note = misc_feature - Xaa is Asn or Tyr
  VAR_SEQ 9: note = misc_feature - Xaa is Thr or Ile
  VAR_SEQ 12: note = misc_feature - Xaa is Ala or Asn
  VAR_SEQ 16: note = misc_feature - Xaa is Gln or Lys
  VAR_SEQ 17: note = misc_feature - Xaa is Gly or Asp
XIXPSTDXXEYXQKFXX

SEQ ID NO: 78 - protein, 17 aa, Mus musculus
YIKPSTDNTEYAQKFQG

SEQ ID NO: 79 - protein, 10 aa, Mus musculus
CARDYYGFDY

SEQ ID NO: 80 - protein, 7 aa, Mus musculus
DYYGFDY

SEQ ID NO: 81 - protein, 18 aa, Mus musculus
CARGGNYLRESYYYAMDY

SEQ ID NO: 82 - protein, 16 aa, Mus musculus
RGGNYLRESYYYAMDY

SEQ ID NO: 83 - protein, 10 aa, Mus musculus
CSKDRGYFDY

SEQ ID NO: 84 - protein, 7 aa, Mus musculus
DRGYFDY

SEQ ID NO: 85 - protein, 18 aa, Mus musculus
RARGDYYGSSRYYYAMDY

SEQ ID NO: 86 - protein, 16 aa, Mus musculus
RGDYYGSSRYYYAMDY

SEQ ID NO: 87 - protein, 13 aa, Mus musculus
CARSYGNYPLMDY

SEQ ID NO: 88 - protein, 10 aa, Mus musculus
SYGNYPLMDY

SEQ ID NO: 89 - protein, 13 aa, Mus musculus
CVRSYGNYPLMDY

SEQ ID NO: 90 - protein, 13 aa, Mus musculus
CARSYGNFPLMDY

SEQ ID NO: 91 - protein, 11 aa, Mus musculus
RSYGNFPLMDY

SEQ ID NO: 92 - protein, 13 aa, Mus musculus
  VAR_SEQ 2: note = misc_feature - Xaa is Ala or Val
  VAR_SEQ 8: note = misc_feature - Xaa is Phe or Tyr
CXRSYGNXPLMDY

SEQ ID NO: 93 - protein, 11 aa, Mus musculus
  SITE 6: note = misc_feature - Xaa is Phe or Tyr
RSYGNXPLMDY

SEQ ID NO: 94 - protein, 117 aa, Mus musculus
QVQLKESGPGLVAPSQSLSITCTVSGFSLTNYGVNWVRQPPGKGLEWLGLIWGGGDTDYNSALKSRLSISKDNSKSQVFLKMETNSLQTDDTARYYCARDYYGFDYWGQGTTLTVSS

SEQ ID NO: 95 - protein, 118 aa, Mus musculus
EVKLVESGGGLVQPGGSLRLSCATSGFTFTDYYMSWVRQPPGKALEWLGFIRNKANGYTTKYSASVKGRFTISRDNSQSILYLQMNTLRAEDSATYYCSKDRGYFDYWGQGTTLTVSS

SEQ ID NO: 96 - protein, 127 aa, Mus musculus
VNSEVQLQQSGAELVRPGALVKLSCKASGFNINDYFMHWVKQRPEQGLEWIGWIDPENGNTIYDPKFQGKASITADTSSNTAYLQLSSLTSEDTAVYYCARGGNYLRESYYYAMDYWGQGTSVTVSS

SEQ ID NO: 97 - protein, 127 aa, Mus musculus
VLSEVLLQQSGPELVKPGASVKIPCKASGYTFTDYNMDWVKQSHGKSLEWIGDINPNNGGTIYNQKFKGKATLTVDKSSSTAYMELRSLTSEDTAVYYRARGDYYGSSRYYYAMDYWGQGTSVTVSS

SEQ ID NO: 98 - protein, 119 aa, Mus musculus
QVQLQQSGAELAKPGASVKMSCRASGYTFTSYWMHWVKQRPGQGLDWIGYIKPSTDNTEYNQKFKDKATLTADKSSSTAYMQLSSLTSEDSAVYYCARSYGNYPLMDYWGQGTSVTVSS

SEQ ID NO: 99 - protein, 119 aa, Mus musculus; identical to SEQ ID NO: 98

SEQ ID NO: 100 - protein, 119 aa, Mus musculus
QVQLQQSGAELAKPGASVKMSCKASGYTFTSYWMHWVKQRPGQGLEWIGYINPSTDYTEYNQKFKDKATLTADKSSTTAYMQLSSLTSEDSAVYYCVRSYGNYPLMDYWGQGTSVTVSS

SEQ ID NO: 101 - protein, 119 aa, Mus musculus; identical to SEQ ID NO: 100

SEQ ID NO: 102 - protein, 119 aa, Mus musculus
QVQLQQSGAELAKPGASVKMSCKASGYTFTSYWMHWVKQRPGQGLEWIGYINPSTDYIEYNQKFKDKATLTAGKSSSTAYMQLSSLTSEDSAVYYCARSYGNFPLMDYWGQGTSVTVSS

SEQ ID NO: 103 - protein, 119 aa, Mus musculus
  VAR_SEQ 23: note = misc_feature - Xaa is Lys or Arg
  VAR_SEQ 46: note = misc_feature - Xaa is Glu or Asp
  VAR_SEQ 52: note = misc_feature - Xaa is Lys or Asn
  VAR_SEQ 57: note = misc_feature - Xaa is Tyr or Asn
  VAR_SEQ 58: note = misc_feature - Xaa is Thr or Ile
  VAR_SEQ 77: note = misc_feature - Xaa is Ser or Thr
  VAR_SEQ 97: note = misc_feature - Xaa is Ala or Val
  VAR_SEQ 103: note = misc_feature - Xaa is Tyr or Phe
QVQLQQSGAELAKPGASVKMSCXASGYTFTSYWMHWVKQRPGQGLXWIGYIXPSTDXXEYNQKFKDKATLTADKSSXTAYMQLSSLTSEDSAVYYCXRSYGNXPLMDYWGQGTSVTVSS

SEQ ID NO: 104 - protein, 119 aa, synthetic construct; identical to SEQ ID NO: 98
  REGION 1..119: note = Humanized Heavy Chain

SEQ ID NO: 105 - protein, 119 aa, synthetic construct
  REGION 1..119: note = Humanized Heavy Chain
QVQLQQSGAEVKKPGASVKVSCKASGYTFTSYWMHWVKQRPGQGLDWIGYIKPSTDNTEYNQKFKDRVTLTADKSTSTAYMQLSNLISEDTAVYYCARSYGNYPLMDYWGQGTSVTVSS

SEQ ID NO: 106 - protein, 119 aa, synthetic construct
  REGION 1..119: note = Humanized Heavy Chain
QVQLVQSGAEVAKPGASVKMSCKASGYTFTSYWMHWVKQAPGQGLDWIGYIKPSTDNTEYNQKFKDKATLTADKSTSTAYMQLSSLRSEDTAVYYCARSYGNYPLMDYWGQGTTVTVSS

SEQ ID NO: 107 - protein, 119 aa, synthetic construct
  REGION 1..119: note = Humanized Heavy Chain
QVQLVQSGAEVKKPGASVKVSCKASGYTFTSYWMHWVKQAPGQGLDWMGYIKPSTDNTEYAQKFQGRVTLTADKSTSTAYMELSSLRSEDTAVYYCARSYGNYPLMDYWGQGTTVTVSS

SEQ ID NO: 108 - protein, 119 aa, synthetic construct
  REGION 1..119: note = Humanized Heavy Chain
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSS

SEQ ID NO: 109 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 110 - protein, 1382 aa, Rattus norvegicus
MKAPTALAPGILLLLLTLAQRSHGECKEALVKSEMNVNMKYQLPNFTAETPIHNVVLPGHHIYLGATNYIYVLNDKDLQKVSEFKTGPVVEHPDCFPCQDCSSKANVSGGVWKDNVNMALLVDTYYDDQLISCGSVNRGTCQRHVLPPDNAADIQSEVHCMFSPLAEEESGQCPDCVVSALGAKVLLSEKDRFINFFVGNTINSSYPPDYSLHSISVRRLKETQDGFKFLTDQSYIDVLPEFRDSYPIKYIHAFESNHFIYFLTVQKETLDAQTFHTRIIRFCSVDSGLHSYMEMPLECILTEKRRKRSTREEVFNILQAAYVSKPGANLAKQIGASPYDDILYGVFAQSKPDSAEPMNRSAVCAFPIKYVNDFFNKIVNKNNVRCLQHFYGPNHEHCFNRTLLRNSSGCEVRSDEYRTEFTTALQRVDLFMGRLNHVLLTSISTFIKGDLTIANLGTSEGRFMQVVLSRTAHFTPHVNFLLDSYPVSPEVIVEHPSNQNGYTLVVTGKKITKIPLNGLGCGHFQSCSQCLSPPYFIQCGWCHNRCVHSNECPSGTWTQEICLPAVYKVFPTSAPLEGGTMLTICGWDFGFKKNNKFDLRKTKVLLGNESCTLTLSESTTNTLKCTVGPAMSEHFNVSVIVSNSRETTQYSAFSYVDPVITSISPRYGPHAGGTLLTLTGKYLNSGNSRHISIGGKTCTLKSVSDSILECYTPGHTVSAEFPVKLKIDLADRVTSSFSYREDPVVSEIHPTKSFISGGSTITGIGKNLNSVSTPKLVIEVHDVGVNYTVACQHRSSSEIICCTTPSLRQLDLQLPLKTKAFFLLDGILSKHFDLTYVHDPMFKPFEKPVMISMGNENVVEIKGDDIDPEAVKGEVLKVGNKSCENLHWHSEALLCTVPSDLLKLNGGELNIEWKQAVSSTVLGKVIVQPDQNFAGLIIGAVSISVVVLLVSGLFLWLRKRKHKDLGSELVRYDARVHTPHLDRLVSARSVSPTTEMVSNESVDYRATFPEDQFPNSSQNGACRQVQYLLTDLSPILTSGDSDISSPLLQNTVHIDLSALNPELVQAVPHVVIGPSSLIVHFNEVIGRGHFGCVYHGTLLDSDGKKIHCAVKSLNRITDIEEVSQFLTEGIIMKDFSHPNVLSLLGICLRSEGSPLVVLPYMKHGDLRNFIRNETHNPTVKDLIGFGLQVAKGMKYLVSKKFVHRDLAARNCMLDEKFTVKVADFGLARDMYDKEYYSVHNKTGAKLPVKWMALESLQTQKFTTKSDVWSFGVLLWELMTRGAPPYPDVNTFDITIYLLQGRRLLQPEYCPDALYEVMLKCWHPKAEMRPSVSELVSRISSIFSTFIGEHYVHVNATYVNVKCVAPYPSLLPSQDNIDGEANT

SEQ ID NO: 111 - protein, 1379 aa, Mus musculus
MKAPTVLAPGILVLLLSLVQRSHGECKEALVKSEMNVNMKYQLPNFTAETPIQNVVLHGHHIYLGATNYIYVLNDKDLQKVSEFKTGPVLEHPDCLPCRDCSSKANSSGGVWKDNINMALLVDTYYDDQLISCGSVNRGTCQRHVLPPDNSADIQSEVHCMFSPEEESGQCPDCVVSALGAKVLLSEKDRFINFFVGNTINSSYPPGYSLHSISVRRLKETQDGFKFLTDQSYIDVLPEFLDSYPIKYIHAFESNHFIYFLTVQKETLDAQTFHTRIIRFCSVDSGLHSYMEMPLECILTEKRRKRSTREEVFNILQAAYVSKPGANLAKQIGASPSDDILFGVFAQSKPDSAEPVNRSAVCAFPIKYVNDFFNKIVNKNNVRCLQHFYGPNHEHCFNRTLLRNSSGCEARSDEYRTEFTTALQRVDLFMGRLNQVLLTSISTFIKGDLTIANLGTSEGRFMQVVLSRTAHLTPHVNFLLDSHPVSPEVIVEHPSNQNGYTLVVTGKKITKIPLNGLGCGHFQSCSQCLSAPYFIQCGWCHNQCVRFDECPSGTWTQEICLPAVYKVFPTSAPLEGGTVLTICGWDFGFRKNNKFDLRKTKVLLGNESCTLTLSESTTNTLKCTVGPAMSEHFNVSVIISNSRETTQYSAFSYVDPVITSISPRYGPQAGGTLLTLTGKYLNSGNSRHISIGGKTCTLKSVSDSILECYTPAQTTSDEFPVKLKIDLANRETSSFSYREDPVVYEIHPTKSFISGGSTITGIGKTLNSVSLPKLVIDVHEVGVNYTVACQHRSNSEIICCTTPSLKQLGLQLPLKTKAFFLLDGILSKHFDLTYVHNPVFEPFEKPVMISMGNENVVEIKGNNIDPEAVKGEVLKVGNQSCESLHWHSGAVLCTVPSDLLKLNSELNIEWKQAVSSTVLGKVIVQPDQNFAGLIIGAVSISVVVLLLSGLFLWMRKRKHKDLGSELVRYDARVHTPHLDRLVSARSVSPTTEMVSNESVDYRATFPEDQFPNSSQNGACRQVQYPLTDLSPILTSGDSDISSPLLQNTVHIDLSALNPELVQAVQHVVIGPSSLIVHFNEVIGRGHFGCVYHGTLLDNDGKKIHCAVKSLNRITDIEEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITIYLLQGRRLLQPEYCPDALYEVMLKCWHPKAEMRPSFSELVSRISSIFSTFIGEHYVHVNATYVNVKCVAPYPSLLPSQDNIDGEGNT

SEQ ID NO: 112 - protein, 1382 aa, Canis lupus
MKAPAVLAPGILVLLFTLVQKSYGECKEALVKSEMNVNMKYQLPNFTAETPIQNVVLHKHHIYLGAVNYIYVLNDKDLQKVAEYKTGPVLEHPDCSPCQDCSHKANLSGGVWEDNINMALLVDTYYDDQLISCGSVHRGTCQRHILPPSNIADIQSEVHCMYSSQADEEPSQCPDCVVSALGTKVLISEKDRFINFFVGNTINSSDHPDHSLHSISVRRLKETQDGFKFLTDQSYIDVLPEFRDSYPIKYVHAFESNHFIYFLTVQRETLDAQTFHTRIIRFCSVDSGLHSYMEMPLECILTEKRRKRSTREEVFNILQAAYVSKPGAHLAKQIGANLNDDILYGVFAQSKPDSAEPMNRSAVCAFPIKYVNEFFNKIVNKNNVRCLQHFYGPNHEHCFNRTLLRNSSGCEARNDEYRTEFTTALQRVDLFMGQFNQVLLTSISTFIKGDLTIANLGTSEGRFMQVVVSRSGLSTPHVNFRLDSHPVSPEAIVEHPLNQNGYTLVVTGKKITRIPLNGLGCEHFQSCSQCLSAPPFVQCGWCHDRCVHLEECPTGAWTQEVCLPAIYEVFPTSAPLEGGTVLTVCGWDFGFRRNNKFDLKKTKVFLGNESCTLTLSESTTNMLKCTVGPAVNEHFNISIIISNGRGTAQYSTFSYVDPIITSISPSYGPKNGGTLLTLTGKYLNSGNSRHISMGGKTCTLKSVSDSILECYTPAQATATEFPIKLKIDLANREMNSFSYQEDPIVYAIHPTKSFISGGSTITAVGKNLNSVSVLRMVIDVHETRRNFTVACQHRSNSEIICCTTPSLQQLNLQLPLKTKAFFMLDGIHSKYFDLIYVHNPVFKPFEKPVMISIGNENVLEIKGNDIDPEAVKGEVLKVGNKSCETIYSDSKAVLCKVPNDLLKLNNELNIEWKQAVSSTVLGKVIVQPDQNFTGLIAGVISISTIVLLLLGLFLWLKRKKQIKDLGSELVRYDARVHTPHLDRLVSARSVSPTTEMVSNESVDYRATFPEDQFPNSSQNGSCRQVQYPLTDLSPMLTSGDSDISSPLLQNTVHIDLSALNPELVQAVQHVVIGPSSLIVHFNEVIGRGHFGCVYHGTLLDNDDKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPRAELRPSFSELVSRISAIFSTFIGEHYVHVNATYVNVKCVAPYPSLLSSQDNIDGEGDT

SEQ ID NO: 113 - protein, 1381 aa, Macaca mulatta
MKAPAVLVPGILVLLFTLVQRSNGECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDT

SEQ ID NO: 114 - protein, 445 aa, synthetic construct
  REGION 1..445: note = Heavy Chain Sequence of hD12
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 115 - protein, 215 aa, synthetic construct
  REGION 1..215: note = Light Chain sequence of hD12
QIVLTQSPAILSLSPGERATLSCSASSSVTSNYLYWYQQKPGSSPKLLIYSTSNLASGVPARFSGSGSGTSYTLTISSLEAEDAASYFCHQWSSYPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 445 aa, synthetic construct
  REGION 1..445: note = Heavy Chain sequence of hD12 T289C
QVQLVQSGAEVKKPGASVKVSCKASGYTFTSYWMHWVKQAPGQGLDWIGYIKPSTDNTEYNQKFKDKATLTADKSTSTAYMELSSLRSEDTAVYYCARSYGNYPLMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKCKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK